(19) United States
(12) Patent Application Publication
(10) Pub. No.: US 2011/0314569 A1
(43) Pub. Date: Dec. 22, 2011

(54) Combination of Two Genetic Elements for Controlling the Floral Development of a Dicotyledonous Plant, and Use in Detection and Selection Methods

(76) Inventors: Abdelhafid Bendahmane, Le Coudray Montceaux (FR); Adnane Boualem, Cusset (FR); Christelle Troadec, Ivry-sur-Seine (FR); Martin Antoine, Gif sur Yvette (FR); Catherine Dogimont, Aubignan (FR)

(21) Appl. No.: 13/056,301

(22) PCT Filed: Jul. 27, 2009
(86) PCT No.: PCT/FR09/51510

§ 371 Date: Jan. 27, 2011

(30) Foreign Application Priority Data
Jul. 28, 2008 (FR) 0855146

(51) Int. Cl. A01H 5/00; A01H 1/06; C07H 21/04; G01N 33/53; C12N 5/10; C12N 15/82

(52) U.S. Cl. 800/276; 435/419; 435/320.1; 536/24.3; 536/24.33; 436/501; 536/23.6; 800/298; 800/290

(57) ABSTRACT

The present invention relates to a combination of two genetic elements for controlling the development of the floral type of a dicotyledonous plant, said combination comprising, respectively:
a first genetic control element (A/a) present in a dicotyledonous plant, in the form of a dominant allele (A), and of a recessive allele (a), and
a second genetic control element (G/g) present in a dicotyledonous plant, in the form of a dominant allele (G), and of a recessive allele (g),
   it being understood that at least the second genetic control element was introduced artificially into said dicotyledonous plant.

The above combination makes it possible to control and/or modify the sex of the flowers of dicotyledonous plants.

### FIELD OF THE INVENTION

The present invention relates to the field of selection of plant varieties, and in particular to selection of the sexual type of plants. It relates to the genotypic detection of the sex of plants by analysis of the polymorphism of a gene A and of a gene G, as well as to means of application of said detection and to methods of obtaining plants whose sexual phenotype is modified.

### PRIOR ART

The production of hybrid plants is of great interest in agronomy and in agriculture. In fact, hybrid plants, owing to the phenomenon of heterosis, also called hybrid vigour, display superiority for many characters, relative to the average of their two parents. This superiority may be reflected for example in better vigour, better yield, greater adaptation to the medium in which the hybrid is cultivated, and great uniformity of the hybrid relative to its parents. This hybrid vigour is even greater when the parents are more distant genetically.

The creation of pure and stable lines, the future parents of the hybrid, is an indispensable step for creating homogeneous and reproducible hybrid varieties expressing the greatest heterosis. It is therefore necessary to create lines that are pure and stable, and then cross these lines to obtain hybrids.

The creation of pure lines involves the self-fertilization of a plant so as to obtain plants having one and the same germ plasm, fixed for all of the required characters of productivity, regularity of yield, or of resistance to diseases.

To create pure lines, it is therefore necessary to use plants whose sexual type permits self-fertilization, for example hermaphroditic plants.

Now, many dicotyledonous plants, and in particular the Cucurbitaceae, can be monoecious, andromonoecious, gynoecious or hermaphroditic.

A first technique employed for obtaining pure lines consists of chemical treatment of the plants so as to obtain plants that can self-fertilize, for example hermaphroditic plants.

In the melon (Cucumis melo) for example, spraying of inhibitors of ethylene synthesis, such as silver nitrate or silver thiosulphate, leads to temporary appearance of stamens in female flowers (Rudich et al., 1969; Risser et al., 1979). In this way, the transformation of gynoecious plants into hermaphroditic plants is used for maintaining pure lines.

However, the production of pure lines by this method is limited by the cost of the chemicals, their duration of action, and their phytotoxic effects. Moreover, such agents may not be effective with regard to production of hybrids from plants with a long flowering time, as new flowers that appeared after the treatment might not be affected by the chemical treatment.

There is therefore a need for a system that would make it possible to control the development of the floral type of a dicotyledonous plant, and obtain a plant of a defined floral type.

Moreover, numerous crossings are necessary to obtain interesting hybrids, from pure lines, and at each crossing, the seedlings with the most promising phenotype are retained.

When crossing pure lines with one another, it is essential to be able to choose the direction of crossing that is carried out, and avoid self-pollination of the plants, which would lead to plants that do not have the required hybrid vigour.

Once again, owing to the diversity of the sexual type of dicotyledonous plants, it is necessary to separate the male flowers and the female flowers of one and the same seedling to prevent self-pollination.

A first technique, employed notably for maize, consists of using mechanical means for emasculating the plants. However, this technique proves extremely expensive since it requires emasculation of each plant whose self-pollination is to be prevented, for each crossing performed.

Another technique consists of carrying out chemical emasculation of the plants, blocking the formation of viable pollen. Thus, in the melon (Cucumis melo), treatment of monoecious plants with Ethrel (an ethylene precursor) leads to temporary disappearance of the male flowers.

Chemicals called gametocides, which are used for producing transient male sterility, have several drawbacks, such as high cost or considerable toxicity, as was mentioned above.

The mechanical or chemical techniques for controlling the floral type described above therefore prove very expensive, especially as numerous crossings are necessary to obtain hybrid plants that have the required characters and can be marketed.

To facilitate the creation of pure lines and of hybrids, there is therefore also a need for a system that would make it possible to control the development of the floral type of a dicotyledonous plant, and obtain a plant of a defined floral type.

Another way of obtaining plants capable of self-pollination that can be used for creating pure lines, or not capable of self-pollination, for creating hybrids, could consist respectively of selecting exclusively hermaphroditic individuals, or exclusively female, present in one species. However, such a technique would also prove to be extremely expensive, as it would necessitate cultivating a very large number of plants, up to the moment when it is possible to determine their sexual type. This technique would moreover be random, as the mechanisms of sex determination of flowers depend notably on environmental factors.

According to yet another route, there have been attempts to identify and characterize the genetic determinants involved in controlling the floral type in the melon. In the melon, genetic control of sex determination is governed by two main genetic determinants, respectively (1) the andromonoecious genetic determinant (“a”) and (2) the gynoecious genetic determinant (“g”), each determinant possessing at least two alleles, and whose combinations produce a great variety of sexual phenotypes. The PCT international application published under No. WO2007/125264 describes the identification and characterization of the genetic determinant (a), which was found to consist of a gene encoding an aminocyclopropane carboxylate synthase (ACS). Thus, PCT application No. WO 2007/125264 provides means for detection and control making it possible to select or generate dicotyledonous plants possessing the dominant allele (A) or the recessive allele (a). The genetic determinant (g) remained completely unknown. At the very most, preliminary data would suggest that the genetic determinant (g), of unknown nature and structure, could be located in a broad genomic region of more than 2.4 megabases delimited by markers designated M8 and M30. Since it is commonly assumed that there are on average 12 open reading frames (“ORFs”) in 100 kilobases of plant genome, the genomic region delimited between markers M8 and M30 was likely to contain about 300 open reading frames.

However, in the absence of characterization of the second gynoecious genetic determinant (or “g”), it was not possible for means to be made available to the public for selection or control of development of the floral type, making it possible for example to discriminate or to generate a population of strictly female plants, since this phenotype is controlled exclusively by the genetic determinant (g). Moreover, selecting or obtaining a population of exclusively hermaphroditic plants would only be possible after identification and characterization of the genetic determinant (g).

There is therefore also a need for a method that would make it possible to select dicotyledonous plants, for example hermaphroditic or female, but without having to cultivate them.

This method should make it possible to select plants that can be used in particular for producing pure lines or hybrids, as was stated above.

### SUMMARY OF THE INVENTION

According to the invention, the gynoecious genetic determinant (g) has been identified and characterized for controlling the floral development of a dicotyledonous plant, which is an angiosperm, and more precisely a plant of the family Cucurbitaceae.

The identification and characterization of the gynoecious genetic determinant (g) made it possible for the first time to develop a combination of the two genetic determinants, andromonoecious (a) and gynoecious (g), allowing complete control of the development of the floral type of a dicotyledonous plant, regardless of the sexual phenotype under consideration.

The invention therefore supplies a combination of the two genetic elements (A/a) and (G/g) that makes it possible to control the development of the floral type of a dicotyledonous plant, in particular of a member of the Cucurbitaceae such as the melon.

It had already been shown in the prior art that, physiologically, the two alleles (A) and (a) differed from one another by different levels of enzymatic activity of a protein, aminocyclopropane carboxylate synthase, also designated ACS.

The inventors have now shown that, physiologically, the two alleles (G) and (g), which were identified and characterized according to the invention, differ from one another by different levels of a new protein, the protein CmWIP1.

The invention therefore relates to the combination of two genetic elements for controlling the development of the floral type of a dicotyledonous plant, said combination comprising respectively:
a) a first genetic control element (A/a) present in said dicotyledonous plant, in the form of a dominant allele (A), and of a recessive allele (a), in which:
the dominant allele (A) consists of a nucleic acid (NA) permitting expression of the protein ACS (aminocyclopropane carboxylate synthase),
the recessive allele (a) differs from the dominant allele by a nucleic acid (NA) that is non-functional in said dicotyledonous plant, and
b) a second genetic control element (G/g) present in said dicotyledonous plant, in the form of a dominant allele (G), and of a recessive allele (g), in which:
the dominant allele (G) consists of a nucleic acid (NG) permitting expression of the protein CmWIP1,
the recessive allele (g) differs from the dominant allele by a nucleic acid (NG) that is non-functional in said dicotyledonous plant,
   it being understood that at least the second genetic control element was introduced artificially into said dicotyledonous plant.

In certain embodiments of the combination according to the invention, the second genetic control element (G/g), the respective characteristics of the dominant allele (G) and of the recessive allele (g) are as follows:
the dominant allele (G) consists of a nucleic acid (NG) comprising:
(i) a regulatory polynucleotide (PG) that is functional in a dicotyledonous plant, and
(ii) a nucleic acid whose expression is regulated by the regulatory polynucleotide (PG), said nucleic acid coding for the protein CmWIP1,
the recessive allele (g) differs from the dominant allele (G) by:
(i) a nucleic acid (NG) not present in the plant, or
(ii) a non-functional regulatory polynucleotide (Pg) in a dicotyledonous plant, or
(iii) a non-functional nucleic acid (Ng) for the expression of an active protein CmWIP1.

The protein ACS comprises the protein of sequence SEQ ID No. 3 or a protein having at least 90% amino acid identity, preferably at least 91%, 92%, 93%, 94%, 95%, 96%, 97%, 98% or 99% amino acid identity, with the protein of sequence SEQ ID No. 3.

The protein CmWIP1 comprises the protein of sequence SEQ ID No. 12 or a protein having at least 90% amino acid identity, preferably at least 91%, 92%, 93%, 94%, 95%, 96%, 97%, 98% or 99% amino acid identity, with the protein of sequence SEQ ID No. 12. The protein CmWIP1 can also comprise the protein of sequence SEQ ID No. 16 or a protein having at least 90% amino acid identity, preferably at least 91%, 92%, 93%, 94%, 95%, 96%, 97%, 98% or 99% amino acid identity, with the protein of sequence SEQ ID No. 16.

In certain embodiments of the combination according to the invention, the second genetic control element (A/a), the respective characteristics of the dominant allele (A) and of the recessive allele (a) are as follows:
the dominant allele (A) consists of a nucleic acid (NA) comprising:
(i) a regulatory polynucleotide (PA) that is functional in a dicotyledonous plant, and
(ii) a nucleic acid whose expression is regulated by the regulatory polynucleotide (PA), said nucleic acid coding for the protein ACS (aminocyclopropane carboxylate synthase),
the recessive allele (a) differs from the dominant allele (A) by:
(i) a nucleic acid (NA) not present in the plant, or
(ii) a regulatory polynucleotide (Pa) non-functional in a dicotyledonous plant, or
(iii) a nucleic acid (Na) non-functional for the expression of an active protein ACS.

The invention also relates to the regulatory polynucleotides (PG) and (Pg) as such.

The invention also relates to methods for obtaining a transformed plant whose sexual phenotype has been modified, as well as the parts of such a plant, notably its seeds.

The invention further relates to the protein CmWIP1 as defined in more detail below, or a fragment of this protein, as well as antibodies directed against the protein CmWIP1.

The invention also relates to methods for detecting the presence of the alleles (A), (a), (G), and (g) in a sample.

### DESCRIPTION OF THE DRAWINGS

FIG. 1 shows the positional cloning of the locus g.

FIG. 1A shows the physical and genetic maps of the locus g on chromosome 4. The locus is bounded by two markers M261 and M335. The broken lines indicate the position of these genetic markers in the various clones BAC.

FIG. 1B shows the representation of the 8 open reading frames or ORFs (broad arrow) found in BAC 102, with their prediction of orientation. The triangular mark represents insertion of the DNA transposon called Gyno-hAT whose sequence is described in SEQ ID No. 14. It is the insertion of this transposon that induced methylation of the promoter of the CmWIP1 gene and led to its inactivation.

FIG. 1C is a high-resolution mapping of the two critical events of recombination in proximity to the locus g. The polymorphisms SNP are indicated and the recombinants P63.2 and 87.94 determined a final region of 1.4 kb.

FIG. 2 shows the results of amplification by semi-quantitative PCR sensitive to the endonuclease McrBr on the DNA of the transposon inserted at the locus g.

The sense primer is located in the sequence of the transposon, and the antisense primer is located on the genomic sequence bordering the transposon. Amplification by PCR without digestion by the endonuclease of the genomic DNA Gynadou resulted in strong amplification, signifying the presence of the transposon at this locus. Amplification by PCR with predigestion by the endonuclease McrBC does not show any amplification, which indicates a very high level of methylation of the transposon. No amplification was obtained for PI124112 whatever the support, because the transposon is not inserted in the locus G.

FIG. 3 shows analysis of methylation at locus g.

FIG. 3A shows amplification by McrBR-sensitive semi-quantitative PCR on the three open reading frames or ORFs closest to the locus g for the monoecious genotype (G−) PI124112 and the gynoecious genotype Gynadou (gg). The absence of amplification with predigestion with McrBC indicates the presence of methylation of the DNA. The oligonucleotides were designed to generate an amplicon which borders the transcription initiation site predicted for each ORF, including a portion of the promoter and a portion of the first exon.

FIG. 3B shows the methylation of the DNA and the structure of the CmWIP1 gene. The black arrows represent the transcription initiation site determined by the “5′ RACE” technique, the black boxes represent the two exons, the symbol after the second exon represents the end of the sequence “3′UTR”, determined by the “TRACE” technique.

The insertion of the transposon is symbolized on the region at the 3′ end of the gene. The methylation of the DNA in the complete sequence of CmWIP1 was determined by amplification by McrBC-sensitive quantitative PCR. Each value corresponds to the mean value from at least three plants, each PCR reaction having been performed in triplicate.

FIG. 3C shows the analysis of methylation of cytosines by sequencing with bisulphite. Two amplicons corresponding to the highly methylated part of the promoter were amplified after treatment with bisulphite. The percentage of cytosines methylated is indicated by vertical bars.

FIG. 4 shows the analysis of methylation at locus g for different gene pools of C. melo. W1998, Bulgaria 14, Paul and Gynadou are homozygous for allele g. PI161375, Vedrantais and PI124112 are homozygous for allele G.

FIG. 4A shows the experiment by which the insertion of the transposon at locus g was screened by PCR amplification in the various gene pools. The sense primer was located in the sequence of the transposon and the antisense primer was located in the genomic sequence bordering the transposon, in order to verify the presence of the insertion (upper line), where the two primers bordering the insertion site were used in order to verify absence of the transposon (lower line).

FIG. 4A shows the results of McrBr-sensitive semi-quantitative PCR amplification on the CmWIP1 gene in the various gene pools. The absence of amplification after digestion with McrBc indicates the presence of methylation of the DNA.

FIG. 5 shows analysis of the profile of expression of the messenger RNA of CmWIP1.

The levels of expression of CmWIP1 were analysed by quantitative PCR. Each value corresponds to the mean value from at least three plants. The levels of expression of CmWIP1 were normalized with the levels of expression of a ubiquitous gene, the actin gene.

FIG. 6 shows the results of analysis of the levels of expression of CmWIP1 analysed by quantitative PCR in a pool of flower buds up to stage 6. Each value corresponds to the mean value from at least three plants. The levels of expression of CmWIP1 were normalized with the levels of expression of a ubiquitous gene, the actin gene.

FIG. 7 shows observation of the floral phenotypes identified by the TILLING technique. The flowers of the main stem of the mutant S306F like that of the mutant P193L were compared with a wild-type male flower and female flower of the monoecious parent. The flowers of the mutants S306F like that of the mutant P193L clearly show development of the ovary in the fourth spiral. The mutant L77F is a weak mutant.

Ov: ovary; St: stamen.

FIG. 8A shows an alignment of a fragment of the messenger RNA of the ACS gene obtained from a plant having the phenotype (A) [upper line] and from a plant having the phenotype (a) [lower line], and shows a point mutation of a nucleotide differentiating (A) and (a).

FIG. 8B shows an alignment of the amino acid sequences encoded by the nucleic acids in FIG. 8A, including for a plant having the phenotype (A) [upper line] and a plant having the phenotype (a) [second line] and shows a point mutation of an amino acid differentiating (A) and (a).

FIG. 9 shows micrographs of transgenic plants of Arabidopsis thaliana bearing the melon allele A or a. FIGS. 9A and 9B show that the siliques of the plants transformed with allele A (FIG. 9A-Cm-A and FIG. 9B) are shorter than the siliques of the wild plants (Col-0) and of the plants transformed with the allele (a).

### DETAILED DESCRIPTION OF THE INVENTION

According to the present invention, the genetic determinant (G/g) that controls, in combination with the genetic determinant (A/a) previously described in the prior art, the development of the floral type in the Cucurbitaceae, has been identified and characterized.

The identification and characterization of the genetic determinant (G/g) offers a person skilled in the art the possibility, for the first time, of selecting or generating dicotyledonous plants having the desired sexual phenotype, in particular Cucurbitaceae such as the melon having the desired sexual phenotype.

It will be recalled that the allele (A) controls the andromonoecious character of the plants, and the allele (G) controls the gynoecious character of the plants, as shown below in Table 1.

TABLE 1

| Phenotype | Genotype | Type of flowers |
|---|---|---|
| Monoecious | A-G- | Male and female |
| Andromonoecious | aaG- | Male and hermaphroditic |
| Hermaphrodite | aagg | Hermaphroditic |
| Gynoecious | A-gg | Female |

Table 1 shows the correspondence between the genotype and the sexual phenotype of flowers of dicotyledonous plants.

The inventors have now shown that, physiologically, the two alleles (G) and (g), which have been identified and characterized according to the invention, differ by different levels of a new protein, protein CmWIP1.

Using sequence comparisons with known proteins, the inventors showed that the new protein CmWIP1 could be classified in the family of zinc-finger proteins of type WIP, which occur in a great variety of plants, including dicotyledons, monocotyledons, gymnosperms and mosses.

From the genetic standpoint, the inventors showed that the allele (g) differs from the allele (G) by a low level of protein CmWIP1 in the plant, compared with that of a plant bearing an allele (G) or else by the production of a protein CmWIP1 that is mutated relative to the protein CmWIP1 produced by a plant bearing an allele (G).

The inventors also showed that the allele (G) is dominant over the allele (g).

As has already been pointed out, it had already been shown in the prior art that, physiologically, the two alleles (A) and (a) differ by different levels of enzymatic activity of a protein, aminocyclopropane carboxylate synthase, also designated ACS, which is a protein involved in the synthesis of ethylene.

Now, various studies have shown that the genes of the floral biology of the Cucurbitaceae code for proteins involved in the pathway for biosynthesis or regulation of ethylene (Kamachi et al., 1997; Kahana et al., 2000).

It had also been shown previously that the allele (a) differs physiologically from the allele (A) by a low level of enzymatic activity of the protein ACS in the plant, compared with that of a plant bearing an allele (A).

It had also been shown that allele (A) is expressed in the promordia of the carpels and it is this expression that blocks the development of the stamens. The absence of expression of allele A or the expression of a mutated form of the protein obtained from TILLING screenings for example or of the protein obtained from allele “a” bearing the mutation A57V does not block the development of the stamens. Finally, it had also been shown that the allele (A) is dominant over the allele (a).

Without wanting to be bound by any theory, the inventors think that the system controlling the development of the floral type by a combination of alleles of the gene (G/g) and (A/a) can be generalized to the Dicotyledoneae, including the Cucurbitaceae family.

The invention therefore relates to a combination of two genetic elements for controlling the development of the floral type of a dicotyledonous plant, said combination comprising respectively:
a) a first genetic control element (A/a) present in said dicotyledonous plant, in the form of a dominant allele (A), and of a recessive allele (a), in which:
the dominant allele (A) consists of a nucleic acid (NA) permitting expression of the protein ACS (aminocyclopropane carboxylate synthase), preferably the protein ACS of sequence SEQ ID No. 3,
the recessive allele (a) differs from the dominant allele by a nucleic acid (NA) that is non-functional in said dicotyledonous plant, and
b) a second genetic control element (G/g) present in said dicotyledonous plant, in the form of a dominant allele (G), and of a recessive allele (g), in which:
the dominant allele (G) consists of a nucleic acid (NG) permitting expression of the protein CmWIP1, preferably the protein CmWIP1 of sequence SEQ ID No. 12,
the recessive allele (g) differs from the dominant allele by a nucleic acid (NG) that is non-functional in said dicotyledonous plant,
   it being understood that at least the second genetic control element was introduced artificially into said dicotyledonous plant.

The non-functional nucleic acid (NA) that characterizes the allele (a) comprises (i) a nucleic acid coding for a protein different from the protein of SEQ ID No. 3, including the mutated protein A57V, (ii) or any other form of protein that is mutated relative to the protein of sequence SEQ ID No. 3 and leading to an inactive enzyme, (iii) or other non-functional ACS (iv) or an unexpressed allele.

The combination of the two genetic elements that is described above makes it possible to control and/or modify the sex of the flowers of dicotyledonous plants, and is therefore very advantageous, relative to the mechanical systems of control, which are often expensive, or chemical, which are often toxic, used in the prior art.

“Allele” means, in the sense of the present invention, one of the forms of a gene occupying a site or locus on a pair of homologous chromosomes. The alleles of a gene relate to the same genetic trait but may determine different phenotypes.

A dominant allele is an allele whose level of phenotypic expression is much greater than that of the homologous allele (called recessive). The dominance can be complete or partial.

A recessive allele is an allele that is only expressed in the phenotype when the plant receives identical alleles from each of its two parents. Conversely, the expression of the recessive allele is masked if the dominant homologous allele is present.

Thus, the combination defined above exists in the form of different states, each corresponding to a phenotype.

When the first genetic control element (A/a) present in a dicotyledonous plant is in the form of allele (A), the plant is of monoecious or gynoecious phenotype.

When the first genetic control element (A/a) present in a plant is in the form of allele (aa), the plant is of hermaphroditic or andromonoecious phenotype.

When the second genetic control element (G/g) present in a dicotyledonous plant is in the form of the allele (G), the plant is of monoecious or andromonoecious phenotype.

When the second genetic control element (G/g) present in a plant is in the form of allele (gg), the plant is of hermaphroditic or gynoecious phenotype.

The correspondence between alleles and phenotypes is summarized in Table 1.

In certain embodiments of the combination according to the invention, for the second genetic control element (G/g), the respective characteristics of the dominant allele (G) and of the recessive allele (g) are as follows:
the dominant allele (G) consists of a nucleic acid (NG) comprising:
(i) a regulatory polynucleotide (PG) that is functional in a dicotyledonous plant, and
(ii) a nucleic acid whose expression is regulated by the regulatory polynucleotide (PG), said nucleic acid coding for the protein CmWIP1 (“C. melo Zinc Finger Protein”) of sequence SEQ ID No. 12,
the recessive allele (g) differs from the dominant allele (G) by:
(i) a nucleic acid (NG) not present in the plant, or
(ii) a non-functional regulatory polynucleotide (Pg) in a dicotyledonous plant, or
(iii) a non-functional nucleic acid (Ng) for the expression of an active protein CmWIP1.

In certain embodiments of the combination according to the invention, for the first genetic control element (A/a), the respective characteristics of the dominant allele (A) and of the recessive allele (a) are as follows:
the dominant allele (A) consists of a nucleic acid (NA) comprising:
(i) a regulatory polynucleotide (PA) that is functional in a dicotyledonous plant, and
(ii) a nucleic acid whose expression is regulated by the regulatory polynucleotide (PA), said nucleic acid coding for the protein ACS (aminocyclopropane carboxylate synthase) of sequence SEQ ID No. 3,
   the recessive allele (a) differs from the dominant allele (A) by:
      (i) a nucleic acid (NA) not present in the plant, or
      (ii) a regulatory polynucleotide (Pa) non-functional in a dicotyledonous plant, or
      (iii) a nucleic acid (Na) non-functional for the expression of an active protein ACS.

The rest of the description presents variants, or preferred embodiments of the first and second genetic elements of control forming part of the control system according to the invention.

### Genetic Control Element G/q, in the Form of Dominant Allele (G), in the Combination of Two Genetic Elements According to the Invention

In general, the genetic control element G/g, present in a plant in the form of the dominant allele (G), makes it possible to obtain a higher level of protein CmWIP1, relative to the level observed when the allele (G) is not present in said plant.

In the rest of the description, it is considered that a “high level” of protein CmWIP1 corresponds to the average level of protein CmWIP1 measured in a plant comprising the dominant allele (G) in its genome, and that a “low level” of protein CmWIP1 corresponds to the average level of protein CmWIP1 observed in a plant not comprising the dominant allele (G) in its genome.

The dominant allele (G) consists of a nucleic acid (NG) comprising:
(i) a regulatory polynucleotide (PG) that is functional in a dicotyledonous plant, and
(ii) a nucleic acid whose expression is regulated by the regulatory polynucleotide (PG), said nucleic acid coding for the protein CmWIP1 of sequence SEQ ID No. 12.

### Functional Regulatory Polynucleotide (PG)

A functional regulatory polynucleotide or promoter (PG) according to the invention consists of a nucleic acid that permits the expression of the protein CmWIP1 of sequence SEQ ID No. 12 in dicotyledonous plants.

As an example, such a promoter comprises, or consists of, the nucleic acid of sequence SEQ ID No. 13, which is located from the nucleotide in position 1 to the nucleotide in position 2999 of the nucleic acid of the CmWIP1 gene of sequence SEQ ID No. 10.

Thus, in the embodiments of the combination of two genetic elements of the invention in which the second genetic element consists of the allele G, the regulatory polynucleotide (PG) can consist of a polynucleotide that comprises, or that consists of, (i) a nucleotide sequence from nucleotide 1 to nucleotide 2999 of the sequence SEQ ID No. 10 or (ii) a sequence having at least 90% nucleotide identity with the sequence 1-2999 of SEQ ID No. 10 and which is functional or (iii) a fragment of the preceding sequences (i) and (ii) and which is functional.

The invention also relates to the regulatory polynucleotide (PG) as such, defined above, as well as fragments of this nucleic acid, as will be described in more detail in the section headed “Nucleic acids according to the invention”.

A functional regulatory polynucleotide (PG) according to the invention can also consist of a promoter that is known to direct the expression of the nucleic acid sequence coding for the protein CmWIP1 constitutively or tissue-specifically.

A functional regulatory polynucleotide (PG) according to the invention can thus be selected from tissue-specific promoters such as those of the genes of the “MADS box” family of class A B C D and E, as described by Theissen et al., 2001 or any other promoter of homeotic genes.

A functional regulatory polynucleotide (PG) according to the invention can thus be selected from:
the promoter 35S of the cauliflower mosaic virus, or the promoter 19S or advantageously the double constitutive promoter 35S (pd35S), described in the article of Kay et al., 1987;
the actin promoter of ri3 followed by the actin intron of ri3 (pAR-IAR) contained in the pAct1-F4 plasmid described by McElroy et al., 1991;
the constitutive promoter EF-1α of the gene coding for the plant elongation factor described in PCT application No. WO 90/02172 or in the article of AXELOS et al. (1989);
the chimeric superpromoter PSP (NI et al., 1995) constituted of the fusion of three copies of the element of transcriptional activity of the promoter of the gene of octopin synthase of Agrobacterium tumefaciens and of the element of transcription activation of the promoter of the gene of mannopin synthase of Agrobacterium tumefaciens; and
the ubiquitin promoter of sunflower (BINET et al., 1991);
the promoter of ubiquitin 1 of maize (CHRISTENSEN et al., 1996).

A functional regulatory polynucleotide (PG) according to the invention can also consist of an inducible promoter.

Thus, the invention relates to a combination of two genetic elements for controlling the floral type of a dicotyledonous plant, as defined previously, in which the regulatory polynucleotide (PG) is sensitive to the action of an inducing signal, and preferably, in which the regulatory polynucleotide (PG) is an inducible polynucleotide activator of transcription or translation.

When the regulatory polynucleotide that activates transcription or translation is sensitive, directly or indirectly, to the action of an activating inducing signal, it is an “inducible activating” polynucleotide in the sense of the invention.

According to the invention, a regulatory polynucleotide of the “inducible activating” type is a regulatory sequence that is only activated in the presence of an external signal. Said external signal can be the fixation of a transcription factor, and the fixation of a transcription factor can be induced under the action of the activating inducing signal to which the regulatory polynucleotide is directly or indirectly sensitive.

When such a construction of nucleic acids is used in a cellular host, expression of the polynucleotide coding for the protein CmWIP1 according to the invention can be induced by bringing the transformed cellular host in contact with the activating inducing signal to which the activating regulatory polynucleotide is directly or indirectly sensitive.

When looking for absence of expression of the polynucleotide coding for a polypeptide CmWIP1 in this transformed cellular host, it is then sufficient to eliminate or suppress the presence of the activating inducing signal to which the regulatory polynucleotide of transcription or translation is sensitive.

A person skilled in the art will have recourse to his general technical knowledge in the field of regulatory polynucleotides, in particular those active in plants, for defining the constructions that correspond to the definition of the above embodiment.

The regulatory sequence capable of controlling the nucleic acid coding for a protein CmWIP1 can be a regulatory sequence inducible by a particular metabolite, such as:

a regulatory sequence inducible by glucocorticoids as described by AOYAMA et al. (1997) or as described by McNELLYS et al. (1998);

a regulatory sequence inducible by ethanol, such as that described by SALTER et al. (1998) or as described by CADDICK et al. (1998);

a regulatory sequence inducible by tetracycline such as that marketed by the company CLONTECH.

a promoter sequence inducible by a pathogen or by a metabolite produced by a pathogen.

a regulatory sequence of genes of type PR, inducible by salicylic acid or BTH or Aliette (Gorlach et al., 1996, Molina et al., 1998);

a regulatory sequence of the Ecdysone receptor type (Martinez et al., 1999) inducible by tebufenozide (product reference RH5992, marketed by ROHM & HAAS) for example, belonging to the dibenzoylhydrazines family.

### Nucleic Acids Coding for the Protein CmWIP1

Preferably, the nucleic acid coding for the protein CmWIP1 comprises, from the 5′ end to the 3′ end, at least:
(i) one sequence having at least 95% identity with the polynucleotide from nucleotide 3000 to nucleotide 3617 of the sequence SEQ ID No. 10, and
(ii) one sequence having at least 95% identity with the polynucleotide from nucleotide 5458 to nucleotide 5901 of the sequence SEQ ID No. 10.

In other embodiments, the nucleic acid coding for the protein CmWIP1 comprises the nucleotide sequence SEQ ID No. 11.

### Genetic Control Element G/g, in the Form of the Recessive Allele (q) of the System According to the Invention

In general, the genetic control element (G/g) in the form of the recessive allele (g), when it is present in a plant that does not possess the dominant allele (G) in its genome, does not allow a level of protein CmWIP1 to be obtained that is as high as that obtained when the allele (G) is present.

The allele (g) can therefore be defined as any alteration of the genotype corresponding to the allele (G), not making it possible to obtain a level of CmWIP1 as high as the allele (G).

The recessive allele (g) differs from the dominant allele (G) by:

(i) a nucleic acid (NG) not present in the plant, or

(ii) a regulatory polynucleotide (Pg) non-functional in a dicotyledonous plant, or

(iii) a nucleic acid (Ng) non-functional for the expression of an inactive protein CmWIP1.

As an example, one embodiment of a control element G/g in the form of recessive allele (g) is illustrated by the nucleic acid of sequence SEQ ID No. 15, which is an allele of the sequence encoding CmWIP1 in which a transposon nucleic acid designated “Gyno-hAT” is present. In the nucleic acid of sequence SEQ ID No. 15, the Gyno-hAT transposon is located from the nucleotide in position 7167 to the nucleotide in position 15412 of the sequence SEQ ID No. 15.

In the nucleic acid of sequence SEQ ID No. 14, the Gyno-hAT transposon is located from the nucleotide in position 10 to the nucleotide in position 8246 of the sequence SEQ ID No. 14.

### Non-Functional Polynucleotide Regulator (Pg)

A non-functional regulatory polynucleotide (Pg), or promoter according to the invention is a nucleic acid which:
(i) does not permit the expression of the protein CmWIP1 of sequence SEQ ID No. 12 in a host cell, or
(ii) permits the expression of this protein at a low level in comparison with the level observed with the regulatory polynucleotide (PG), or
(iii) permits the expression of the protein CmWIP1 during the life of the plant, for a shorter time, in comparison with that observed with the regulatory polynucleotide (PG).

In certain embodiments of the non-functional regulatory polynucleotide (Pg), said polynucleotide (Pg) is methylated. For example, the regulatory polynucleotide (Pg) can consist of a regulatory polynucleotide (PG) that is in the form of a methylated nucleic acid.

“Methylated nucleic acid” or “methylated regulatory polynucleotide” means, according to the invention, the corresponding nucleic acid for which the ratio (number of methylated bases)/(number of unmethylated bases) is at least 5/1. Thus, according to the invention, a methylated nucleic acid comprises a nucleic acid possessing a ratio (number of methylated bases)/(number of unmethylated bases) of at least, 6/1, 7/1, 8/1, 9/1, 10/1, 11/1, 12/1, 13/1, 14/1, 15/1, 16/1, 17/1, 18/1 and 20/1.

The ratio (number of methylated bases)/(number of unmethylated bases) can easily be determined by a person skilled in the art by any known technique. A person skilled in the art can notably use the method of sequencing with bisulphite, as described in the examples of the present description.

To compare the level of expression of several promoters, a simple technique, known by a person skilled in the art, consists of placing a selection marker gene under the control of the promoters to be tested. A selection marker gene can be for example the gene for resistance to the herbicide BASTA, well known by a person skilled in the art.

Another technique can consist of measuring the level of the protein CmWIP1 obtained when the sequence coding for this protein is under the control of different promoters, using antibodies directed against this protein, and the methods described in the section “polypeptides according to the invention”.

As shown in the examples, an illustration of a non-functional promoter (Pg) consists of a promoter having a nucleotide sequence identical to the nucleotide sequence of a functional promoter (PG) but which is in cellulo or in vivo in a non-functional methylated form. In the specific embodiment illustrated in the examples, the methylated state of the promoter (Pg) is caused by the presence of a transposable element (TE) located at a distance of less than 1 kilobase from the 3′ end of the CmWIP1 gene.

A non-functional polynucleotide (Pg) can also be any polynucleotide derived from the polynucleotide (PG) as defined above whose nucleotide sequence comprises an insertion, a substitution or a deletion of one or more nucleotides, relative to the nucleotide sequence of the regulatory polynucleotide.

Thus, the invention also relates to a nucleic acid comprising a nucleotide sequence bearing at least one alteration selected from a mutation, an insertion or a deletion, relative to the nucleic acid from nucleotide 1 to nucleotide 2999 of the sequence SEQ ID No. 10, said altered nucleic acid leading to reduced expression of the protein CmWIP1, when it controls the expression of said protein, relative to the expression of the protein CmWIP1 controlled by the nucleic acid from nucleotide 1 to nucleotide 2999 of the sequence SEQ ID No. 10.

The invention also relates to the regulatory polynucleotide (Pg) as such, as defined above.

The invention also relates to a nucleic acid comprising a regulatory polynucleotide (Pg) and a nucleic acid coding for the protein CmWIP1 of sequence SEQ ID No. 12.

The invention also relates to a combination of two genetic elements for controlling the development of the floral type of a dicotyledonous plant as defined generally in the present description, in which the regulatory polynucleotide (Pg) is sensitive to the action of an inducing signal, and preferably, in which the regulatory polynucleotide (Pg) is an inducible repressor polynucleotide of transcription or translation.

“Repressor” regulatory polynucleotide means, according to the invention, a regulatory sequence whose constitutive activity can be blocked by an external signal. Said external signal can be the absence of fixation of a transcription factor recognized by the repressor regulatory polynucleotide. The absence of fixation of the transcription factor can be induced under the action of the repressor inducing signal to which the repressor regulatory polynucleotide is sensitive.

According to this first particular embodiment, expression of the sequence coding for a protein CmWIP1 is constitutive in the cellular host selected, in the absence of the repressor inducing signal to which the repressor regulatory polynucleotide is directly or indirectly sensitive.

Bringing the cellular host in contact with the repressor inducing signal has the effect, owing to a direct or indirect action on the repressor regulatory polynucleotide, of inhibiting and/or blocking the expression of the polynucleotide coding for the protein CmWIP1.

For effecting the DNA constructions according to the invention comprising a repressor regulatory polynucleotide, a person skilled in the art will have recourse to his general technical knowledge in the field of gene expression in plants.

A method of obtaining a transformed plant, employing this type of regulatory polynucleotide, is described in the section headed “methods of obtaining a transformed plant according to the invention”.

### Non-Functional Nucleic Acid (Ng) for the Expression of an Active Protein CmWIP1

A nucleic acid (Ng) includes the nucleic acids comprising at least one portion of a sequence encoding an active protein CmWIP1 but that do not permit, when they are placed under the control of a functional regulatory polynucleotide in cells of dicotyledonous plants, the production of an active protein CmWIP1 in said plants.

A nucleic acid (Ng) essentially includes the nucleic acids (NG) in which one or more mutations are present in at least one intron or one exon, each mutation being selected from (i) substitution of a nucleotide or more than one nucleotide, (ii) deletion of a nucleotide or of at least two consecutive nucleotides and (ii) deletion of a nucleotide or of at least two consecutive nucleotides, relative to the reference nucleic acid (NG). A nucleic acid (Ng) notably includes the nucleic acid coding for an inactive protein CmWIP1.

“Nucleic acid coding for an inactive protein CmWIP1” means, in the sense of the present invention, a nucleic acid that codes for a protein that differs from the protein CmWIP1 of sequence SEQ ID No. 12, by the substitution, deletion, or insertion of one or more amino acids, and that does not possess the biological activity of the protein CmWIP1 of sequence SEQ ID No. 12.

It also includes a nucleic acid that codes for a protein that differs from the protein CmWIP1 of sequence SEQ ID No. 16, by the substitution, deletion, or insertion of one or more amino acids, and that does not possess the biological activity of the protein CmWIP1 of sequence SEQ ID No. 16.

In particular, said inactive protein CmWIP1, when it is expressed in a plant that does not express any active protein CmWIP1, in particular any protein CmWIP1 of sequence SEQ ID No. 12, or protein CmWIP1 of sequence SEQ ID No. 16, induces respectively:
a phenotype of hermaphroditic plant in combination with the presence in homozygous form of the alleles (a/a) in said plant,
a phenotype of female plant in combination with (i) the presence in homozygous form of the alleles (A/a) in said plant or in combination with (ii) the presence in heterozygous form of the alleles (A/a) in said plant.

It is shown in the examples that plants possessing the allele (g) of the genetic element (G/g) were obtained with nucleic acids encoding an inactive protein CmWIP1. Notably, it is shown in the examples that plants possessing the allele (g) of the genetic element (G/g) were obtained with nucleic acids encoding a protein CmWIP1 possessing a substitution of a single nucleotide relative to the nucleotide sequence SEQ ID No. 11 encoding the protein CmWIP1 of sequence SEQ ID No. 12.

For purposes of illustration, the examples show plants possessing the allele (g) and whose corresponding nucleic acid encodes a mutated protein CmWIP1 possessing a substitution of an amino acid selected from L77F, P193L and S306F, according to the numbering of amino acids used for the sequence SEQ ID No. 12.

The genetic control element A/a, in the form of dominant allele (A) or in the form of dominant allele (a) has already been described in French patent application No. FR 2 900 415 and in PCT application No. WO 2007/125264.

However, since the genetic control element (A/a) is an important element of the combination of two genetic elements for controlling the development of the floral type according to the invention, its main characteristics are described again below.

### Genetic Control Element A/a, in the Form of Dominant Allele (A) of the Combination According to the Invention

In general, the genetic control element A/a, present in a plant in the form of the dominant allele (A), makes it possible to obtain a higher level of active protein ACS, relative to the level observed when the allele (A) is not present in said plant.

In the rest of the description, it is considered that a “high level” of the protein ACS corresponds to the average level of the protein ACS measured in a plant comprising the dominant allele (A) in its genome, and that a “low level” of the protein ACS corresponds to the average level of active protein ACS observed in a plant not comprising the dominant allele (A) in its genome. A low level of the protein ACS includes a zero level of active protein ACS, for example in the case of expression of inactive ACS, including the product of the allele “a” bearing the mutation A57V.

The dominant allele (A) consists of a nucleic acid (NA) comprising:
(i) a functional regulatory polynucleotide (PA) in a dicotyledonous plant, and
(ii) a nucleic acid whose expression is regulated by the regulatory polynucleotide (PA), said nucleic acid coding for the protein ACS of sequence SEQ ID No. 3.

### Functional Regulatory Polynucleotide (PA)

A functional regulatory polynucleotide or promoter (PA) according to the invention consists of a nucleic acid that permits expression of the protein ACS of sequence SEQ ID No. 3 in dicotyledonous plants.

As an example, such a promoter comprises a nucleotide sequence from nucleotide 1 to nucleotide 5906 of the sequence SEQ ID No. 1.

Thus, in the embodiments of the combination of two genetic elements of the invention in which the first genetic element consists of the allele A, the regulatory polynucleotide (PA) can consist of a polynucleotide that comprises, or that consists of, (i) a nucleotide sequence from nucleotide 1 to nucleotide 5906 of the sequence SEQ ID No. 1 or (ii) a sequence having at least 90% nucleotide identity with the sequence 1-5906 of SEQ ID No. 1 and which is functional or (iii) a fragment of the preceding sequences (i) and (ii) and which is functional.

Thus, in certain embodiments of the combination of two genetic elements of the invention, the regulatory polynucleotide (PA) comprises or consists of a nucleotide sequence from nucleotide 1 to nucleotide 5906 of the sequence SEQ ID No. 1.

A functional regulatory polynucleotide (PA) that is included in a combination of two genetic elements according to the invention can also consist of a promoter that is known to direct the expression of the nucleic acid sequence coding for the protein ACS constitutively or tissue-specifically.

A functional regulatory polynucleotide (PA) included in a combination of two genetic elements according to the invention can thus be selected from any one of the constitutive or tissue-specific promoters described previously for certain embodiments of the regulatory polynucleotide (PG).

A functional regulatory polynucleotide (PA) according to the invention can also consist of an inducible promoter.

Thus, the invention relates to a combination of two genetic elements as defined above, in which the regulatory polynucleotide (PA) is sensitive to the action of an inducing signal, and preferably, in which the regulatory polynucleotide (PA) is an inducible activating polynucleotide of transcription or translation, which can be selected from any one of the inducible activating polynucleotides described in certain embodiments of the regulatory polynucleotide (PG).

When such a construction of nucleic acids is used in a cellular host, expression of the polynucleotide coding for the protein ACS according to the invention can be induced by bringing the transformed cellular host in contact with the activating inducing signal to which the activating regulatory polynucleotide is directly or indirectly sensitive.

When we are looking for absence of expression of the polynucleotide coding for a polypeptide ACS in this transformed cellular host, it is then sufficient to eliminate or suppress the presence of the activating inducing signal to which the regulatory polynucleotide of transcription or translation is sensitive.

A person skilled in the art will have recourse to his general technical knowledge in the field of regulatory polynucleotides, in particular those active in plants, for defining the constructions corresponding to the definition of the above embodiment, and in particular those described for the regulatory polynucleotide (PG).

### Nucleic Acids Coding for the Protein ACS

Preferably, the nucleic acid coding for the protein ACS comprises, from the 5′ end to the 3′ end, at least:
(i) one sequence having at least 95% identity with the polynucleotide from nucleotide 5907 to nucleotide 6086 of the sequence SEQ ID No. 1,
(ii) one sequence having at least 95% identity with the polynucleotide from nucleotide 6181 to nucleotide 6467 of the sequence SEQ ID No. 1, and
(iii) one sequence having at least 95% identity with the polynucleotide from nucleotide 7046 to nucleotide 7915 of the sequence SEQ ID No. 1.

### Genetic Control Element A/a, in the Form of the Recessive Allele (a) of the Combination According to the Invention

In general, the genetic control element (A/a) in the form of the recessive allele (a), when it is present in a plant that does not possess the dominant allele (A) in its genome, does not allow to obtain a level of the protein ACS as high as that obtained when the allele (A) is present.

We can therefore define the allele (a) as any alteration of the genotype corresponding to allele (A), not allowing an active level of ACS to be obtained.

The recessive allele (a) differs from the dominant allele (A) by:
(i) a nucleic acid (NA) not present in the plant, or
(ii) a regulatory polynucleotide (Pa) non-functional in a dicotyledonous plant, or
(iii) a nucleic acid coding for an inactive protein ACS, or
(iv) a regulatory polynucleotide (Pa) non-functional in a dicotyledonous plant, and a nucleic acid coding for an inactive protein ACS.

### Non-Functional Polynucleotide Regulator (Pa)

A non-functional regulatory polynucleotide (Pa) or promoter according to the invention is a nucleic acid which:
(i) does not permit expression of the protein ACS of sequence SEQ ID No. 3 in a host cell, or
(ii) permits the expression of this protein at a low level in comparison with the level observed with the regulatory polynucleotide (PA), or
(iii) permits expression of the protein ACS during the life of the plant, for a shorter time, in comparison with that observed with the regulatory polynucleotide (PA).

In certain embodiments of the non-functional regulatory polynucleotide (Pa), said polynucleotide (Pa) is methylated. For example, the regulatory polynucleotide (Pa) can consist of a regulatory polynucleotide (PA) that is in the form of a methylated nucleic acid.

“Methylated nucleic acid” or “methylated regulatory polynucleotide” means, according to the invention, the corresponding nucleic acid for which the ratio (number of methylated bases)/(number of unmethylated bases) is at least 5/1. Thus, according to the invention, a methylated nucleic acid includes a nucleic acid possessing a ratio (number of methylated bases)/(number of unmethylated bases) of at least, 6/1, 7/1, 8/1, 9/1, 10/1, 11/1, 12/1, 13/1, 14/1, 15/1, 16/1, 17/1, 18/1 and 20/1.

The ratio (number of methylated bases)/(number of unmethylated bases) can be determined easily by a person skilled in the art by any known technique. A person skilled in the art can notably use the method of sequencing with bisulphite, as described in the examples of the present description.

For comparing the level of expression of several promoters, a simple technique, known by a person skilled in the art, consists of placing a selection marker gene under the control of the promoters to be tested. A selection marker gene can be for example the gene for resistance to the herbicide BASTA, well known by a person skilled in the art.

Another technique can consist of measuring the level of the protein ACS obtained when the sequence coding for this protein is under the control of different promoters, using antibodies directed against this protein, and the methods described in the section “polypeptides according to the invention”.

As an example, a non-functional regulatory polynucleotide (Pa) comprises a nucleotide sequence from nucleotide 1 to nucleotide 3650 of the sequence SEQ ID No. 2.

As another example, a non-functional regulatory polynucleotide (Pa) comprises certain nucleotide sequences comprising one or more substitutions, deletions or additions of bases, relative to the nucleotide sequence from nucleotide 1 to nucleotide 3650 of the sequence SEQ ID No. 1.

Thus, in the combination of two genetic elements for controlling the development of the floral type according to the invention, a non-functional regulatory polynucleotide (Pa) can comprise a nucleotide sequence from nucleotide 1 to nucleotide 3650 of the sequence SEQ ID No. 2 altered by one of the methods known by a person skilled in the art.

The invention also relates to a combination of two genetic elements for controlling the development of the floral type as defined in the present description, and in which the allele (a) is a nucleic acid comprising sequence SEQ ID No. 2. Said nucleic acid of sequence SEQ ID No. 2 comprises a regulatory polynucleotide (Pa) and a nucleic acid coding for the protein ACS of sequence SEQ ID No. 3.

A non-functional polynucleotide (Pa) can also be any polynucleotide derived from the polynucleotide (PA) as defined above whose nucleotide sequence comprises an insertion, a substitution or a deletion of one or more nucleotides, relative to the nucleotide sequence of the regulatory polynucleotide.

Thus, in certain embodiments of the combination according to the invention, a polynucleotide (Pa) consists of a nucleic acid comprising a nucleotide sequence bearing at least one alteration selected from a mutation, an insertion or a deletion, relative to the nucleic acid from nucleotide 1 to nucleotide 5907 of the sequence SEQ ID No. 1, said altered nucleic acid leading to reduced expression of the protein ACS, when it controls the expression of said protein, relative to the expression of the protein ACS controlled by the nucleic acid from nucleotide 1 to nucleotide 5907 of the sequence SEQ ID No. 1.

The invention also relates to a combination of two genetic elements for controlling the development of the floral type as defined above, in which the regulatory polynucleotide (Pa) is sensitive to the action of an inducing signal, and preferably, in which the regulatory polynucleotide (Pa) is an inducible repressor polynucleotide of transcription or translation.

According to this first particular embodiment, expression of the sequence coding for a protein ACS is constitutive in the cellular host selected, in the absence of the repressor inducing signal to which the repressor regulatory polynucleotide is directly or indirectly sensitive.

Bringing the cellular host in contact with the repressor inducing signal has the effect, owing to a direct or indirect action on the repressor regulatory polynucleotide, of inhibiting and/or blocking the expression of the polynucleotide coding for the protein ACS.

For producing the DNA constructions according to the invention comprising a repressor regulatory polynucleotide, a person skilled in the art will have recourse to his general technical knowledge in the field of gene expression in plants.

A method of obtaining a transformed plant, employing this type of regulatory polynucleotide, is described in the section headed “methods of obtaining a transformed plant according to the invention”.

### Non-Functional Nucleic Acid (Na) for the Expression of an Active Protein ACS

A nucleic acid (Na) includes the nucleic acids comprising at least one portion of a sequence encoding an active protein ACS but that do not permit, when they are placed under the control of a functional regulatory polynucleotide in cells of dicotyledonous plants, the production of an active protein CmWIP1 in said plants.

A nucleic acid (Na) includes essentially the nucleic acids (NA) in which one or more mutations are present in at least one intron or one exon, each mutation being selected from (i) substitution of a nucleotide or more than one nucleotide, (ii) deletion of a nucleotide or of at least two consecutive nucleotides and (iii) deletion of a nucleotide or of at least two consecutive nucleotides, relative to the reference nucleic acid (NA). A nucleic acid (Na) includes notably the nucleic acids coding for an inactive protein ACS.

“Nucleic acid coding for an inactive protein ACS” means, in the sense of the present invention, a nucleic acid that codes for a protein that differs from the protein ACS of sequence SEQ ID No. 3, by the substitution, deletion, or insertion of one or more amino acids, and that does not possess the biological activity of the protein ACS of sequence SEQ ID No. 3. An example illustrating such a nucleic acid is presented in FIG. 8.

In particular, an inactive protein ACS of this kind does not permit the transformation of S-adenosyl methionine to ACC (1-aminocyclopropane-1-carboxylate).

### Nucleic Acids According to the Invention

As stated above, two allelic variants (G) and (g) of the second genetic control element (G/g) included in a combination of two genetic elements for control of floral development of the invention have been characterized.

The inventors identified the nucleic acid of sequence SEQ ID No. 10 as being a nucleic acid corresponding to the dominant allelic variant (G) and its methylated form in planta, as corresponding to the recessive allelic variant (g) of the second genetic control element in the form of a gene (G/g).

In the combination of two genetic elements for control of floral development of the invention, at least the second of the two genetic control elements was introduced artificially in a plant.

As was presented above, said introduction causes a change of the sex of the flower of the plant, which is one of the required aims according to the invention.

Accordingly, the nucleic acid of sequence SEQ ID No. 10 forms part of the objects of the invention.

The present invention therefore relates to a nucleic acid comprising a polynucleotide possessing at least 95% nucleotide identity with the nucleotide sequence SEQ ID No. 10, or with a fragment of the sequence SEQ ID No. 10, provided that said nucleic acid possesses the functional characteristics of the allele (G) as defined above.

A nucleic acid of sequence complementary to the nucleic acid as defined above also forms part of the invention.

Another object of the invention is a nucleic acid consisting of a polynucleotide possessing at least 95% nucleotide identity with the sequence SEQ ID No. 10, or with a fragment of the sequence SEQ ID No. 10, or a nucleic acid of complementary sequence, provided that said nucleic acid possesses the functional characteristics of the allele (G) as defined above.

The invention also relates to a nucleic acid comprising at least 12, preferably at least 15 and most preferably at least 20 consecutive nucleotides of the nucleic acid of sequence SEQ ID No. 10, it being understood that said nucleic acid includes in its definition the “fragments” of a nucleic acid according to the invention as defined in the present description.

The invention also relates to the nucleic acid comprising or consisting of the sequence SEQ ID No. 10.

The invention also relates to a nucleic acid comprising at least 12, preferably at least 15 and most preferably at least 20 consecutive nucleotides of the nucleic acid of sequence SEQ ID No. 10, it being understood that said nucleic acid includes in its definition the “fragments” of a nucleic acid according to the invention as defined in the present description.

The allele (G) defined by the sequence SEQ ID No. 10 comprises, from the 5′ end to the 3′ end, respectively:

a) a non-coding sequence bearing regulatory elements of transcription and/or translation of this gene, located upstream of the first exon, from the nucleotide in position 1 to the nucleotide in position 2999 of the sequence SEQ ID No. 10;

b) a so-called “coding” region which comprises the two exons and the intron of the gene (G/g), this coding region being located from the nucleotide in position 3000 to the nucleotide in position 5901 of the sequence SEQ ID No. 10; and

c) a non-coding region located downstream of the coding region, from the nucleotide in position 5902 to the nucleotide in position 7621 of the sequence SEQ ID No. 10.

The details of the structural characteristics of the two exons and of the intron of gene G/g are given below in Table 2. The structural characteristics of the two exons and of the intron of the alleles (G) and (g) of the gene (G/g) are very similar, so that the exons of alleles (G) and (g) can, in certain embodiments, code for one and the same protein of sequence SEQ ID No. 12. As was mentioned above, the main difference between the nucleotide sequences corresponding to the alleles (G) and (g) may occur:
(i) either in the upstream regulatory sequences corresponding to these two alleles, which are non-methylated in planta for the allele (G) and which are methylated in planta for the allele (g),
(ii) or in the sequence of the exons, since a single nucleotide substitution causing the substitution of an amino acid in the sequence of the protein CmWIP1 is sufficient for obtaining the allele (g).

TABLE 2
Sequences of the exons of the gene G/g

| | | Position of the |
|---|---|---|
| | Position of the nucleotide at 5′ on | nucleotide at 3′ on |
| Exon No. | SEQ ID No. 10 (allele G) | SEQ ID No. 10 (allele G) |
| 1 | 3000 | 3617 |
| 2 | 5458 | 5901 |

The invention also relates to a nucleic acid comprising at least 12 consecutive nucleotides of an exon polynucleotide of the gene G/g, such as the polynucleotides 1 and 2 described in Table 2 above, which are included in the nucleic acid of sequence SEQ ID No. 10.

Said nucleic acid codes for at least one part of the protein CmWIP1 and can notably be inserted into a recombinant vector intended for expression of the corresponding product of translation in a host cell or in a plant transformed with this recombinant vector, with a view to obtaining a plant of genotype (G).

Said nucleic acid can also be used for the synthesis of nucleotide probes and primers intended for the detection or amplification of nucleotide sequences comprised in the gene (G/g) in a sample.

The sequences described above can if necessary bear one or more mutations, preferably one or more mutations of a kind to induce the synthesis of an inactive protein CmWIP1, and to modify the sexual type of a plant bearing said mutated gene (G/g). Said sequences comply with the definition of nucleic acids coding for an inactive protein CmWIP1, defined generally above.

TABLE 3
Sequence of the intron of the gene (G/q)

| | | Position of the |
|---|---|---|
| | Position of the nucleotide at 5′ on | nucleotide at 3′ on |
| Intron No. | SEQ ID No. 10 (allele G) | SEQ ID No. 10 (allele G) |
| 1 | 3618 | 5457 |

The invention also relates to a nucleic acid comprising at least 12 consecutive nucleotides of an intron polynucleotide of the gene (G/g), described above in Table 3, which are included in the nucleic acid of sequence SEQ ID No. 10.

Said nucleic acid can be used as oligonucleotide probe or primer for detecting the presence of at least one copy of the gene (G/g) in a sample, or for amplifying a specified target sequence within the gene (A/a).

Said nucleic acid can also be used for amplifying a specified target sequence within the gene (G/g) or inhibiting it by a sense or co-suppression approach, or by the use of double-stranded RNA (Wassenegger et al. 1996; Kooter et al. 1999) for interference. Said nucleic acid can also be used for finding functional allelic variants of the gene (G/g), which can be used in a method of selection of plants possessing a specified sexual type.

### Other Nucleic Acids According to the Invention, Coding for the Protein CmWIP1

The invention also relates to a nucleic acid comprising a polynucleotide possessing at least 95% nucleotide identity with the nucleotide sequence starting at the nucleotide in position 3000 and ending at the nucleotide in position 5901 of the sequence SEQ ID No. 10 as well as a nucleic acid of complementary sequence.

The invention also relates to a nucleic acid possessing at least 95% nucleotide identity with the nucleotide sequence starting at the nucleotide in position 3000 and ending at the nucleotide in position 5901 of the sequence SEQ ID No. 10, as well as a nucleic acid of complementary sequence.

The invention further relates to a nucleic acid comprising the nucleotide sequence starting at the nucleotide in position 3000 and ending at the nucleotide in position 5901 of the sequence SEQ ID No. 10 or a nucleic acid of complementary sequence.

The invention also relates to a nucleic acid consisting of the nucleotide sequence starting at the nucleotide in position 3000 and ending at the nucleotide in position 5901 of the sequence SEQ ID No. 10 or a nucleic acid of complementary sequence.

Another object of the invention consists of a nucleic acid comprising, at least:
(i) one sequence having at least 95% identity with the polynucleotide from nucleotide 3000 to nucleotide 3617 of the sequence SEQ ID No. 10,
(ii) one sequence having at least 95% identity with the polynucleotide from nucleotide 5458 to nucleotide 5901 of the sequence SEQ ID No. 10, and

The invention further relates to a nucleic acid comprising, from the 5′ end to the 3′ end:
(i) a sequence extending from the nucleotide 3000 to the nucleotide 3617 of the sequence SEQ ID No. 10, and
(ii) a sequence extending from the nucleotide 5458 to the nucleotide 5901 of the sequence SEQ ID No. 10.

A nucleic acid coding for the protein CmWIP1 can further comprise conventional leader and terminator sequences, known by a person skilled in the art.

### Products of Transcription and Translation of the Gene (G/g) and Polypeptides According to the Invention

The expression of the genomic nucleic acid encoding the protein CmWIP1 leads to the synthesis of a messenger RNA whose cDNA is the nucleic acid of sequence SEQ ID No. 11, which is also one of the objects of the present invention.

The invention therefore also relates to the polypeptide comprising the amino acid sequence SEQ ID No. 12, also called “protein CmWIP1” in the present description, as well as a polypeptide possessing at least 95% amino acid identity with the sequence SEQ ID No. 12, or a fragment or a variant of the latter.

An illustration according to the invention of a protein CmWIP1 possessing at least 95% amino acid identity with the sequence SEQ ID No. 12 consists of the protein of sequence SEQ ID No. 16, which differs from the protein of sequence SEQ ID No. 12 by the deletion of a serine residue.

A fragment of a protein CmWIP1 according to the invention comprises at least 10, 50, 100, 200, 300, 320, 330, 340, 345 or 353 consecutive amino acids of a polypeptide of sequence SEQ ID No. 12.

The invention also relates to a polypeptide comprising an amino acid sequence having at least 95% amino acid identity with the sequence of a protein CmWIP1 of sequence SEQ ID No. 12.

Advantageously, a polypeptide having at least 96%, 97%, 98%, 99%, 99.1%, 99.2%, 99.3%, 99.4%, 99.5%, 99.6%, 99.7%, 99.8% or 99.9% amino acid identity with the sequence of a polypeptide of sequence SEQ ID No. 12, or a peptide fragment of the latter, also forms part of the invention.

In general, the polypeptides according to the present invention are in an isolated or purified form.

A polypeptide according to the invention can be obtained by genetic recombination according to techniques that are well known by a person skilled in the art, for example techniques described in AUSUBEL et al. (1989).

A polypeptide according to the invention can also be prepared by conventional techniques of chemical synthesis, whether in homogeneous solution or in the solid phase.

As an illustration, a polypeptide according to the invention can be prepared by the technique in homogeneous solution described by HOUBEN WEIL (1974) or by the technique of solid phase synthesis described by MERRIFIELD (1965a; 1965b).

Preferably, the variant polypeptides of a polypeptide according to the invention conserve their capacity to be recognized by antibodies directed against the polypeptides of sequences SEQ ID No. 12.

A polypeptide encoded by the gene (G/g) according to the invention, such as a polypeptide of amino acid sequence SEQ ID No. 12, or a variant or a peptide fragment of the latter is useful notably for preparing antibodies intended for detecting the presence and/or expression of a polypeptide of sequences SEQ ID No. 12 or of a peptide fragment of the latter in a sample.

In addition to detection of the presence of a polypeptide encoded by the gene (G/g) or of a peptide fragment of such a polypeptide in a sample, antibodies directed against these polypeptides are used for quantifying the synthesis of a polypeptide of sequences SEQ ID No. 12, for example in cells of a plant, and thus determine the sex of the plant, but without having to cultivate it.

“Antibodies” means, in the sense of the present invention, notably polyclonal or monoclonal antibodies or fragments (for example the F(ab)′2, F(ab) fragments) or any polypeptide comprising a domain of the initial antibody recognizing the target polypeptide or polypeptide fragment according to the invention.

Monoclonal antibodies can be prepared from hybridomas according to the technique described by KOHLER and MILSTEIN (1975).

The present invention also relates to antibodies directed against a polypeptide as described above or a fragment or a variant of the latter, such as produced in the trioma technique or the hybridoma technique described by KOZBOR et al. (1983).

The invention also relates to single-chain antibody fragments Fv (ScFv) as described in U.S. Pat. No. 4,946,778 or by MARTINEAU et al. (1998).

The antibodies according to the invention also comprise antibody fragments obtained by means of phage banks as described by RIDDER et al. (1995) or humanized antibodies as described by REINMANN et al. (1997) and LEGER et al. (1997). The antibody preparations according to the invention can be used in immunological detection assays intended for identification of the presence and/or amount of a polypeptide of sequences SEQ ID No. 3, or of a peptide fragment of the latter, present in a sample.

An antibody according to the invention can further comprise an isotopic or non-isotopic, for example fluorescent, detectable marker or can be coupled to a molecule such as biotin, according to techniques that are well known by a person skilled in the art.

Thus, the invention further relates to a method for detecting the presence of a polypeptide according to the invention in a sample, said method comprising the stages of:

a) contacting the sample to be tested with an antibody as described above;

b) detecting the antigen/antibody complex that formed.

The invention also relates to a diagnostic kit for detecting the presence of a polypeptide according to the invention in a sample, said kit comprising:

a) an antibody as defined above;

b) if necessary, one or more reagents required for detecting the antigen/antibody complex that formed.

Another object of the invention consists of using a nucleic acid or an allelic variant of a nucleic acid as defined above in plant selection programmes for obtaining plants whose floral type has been modified.

### Nucleic Acids Comprising a Functional Regulatory Polynucleotide (PG)

A functional regulatory polynucleotide or promoter (PG) according to the invention consists of a nucleic acid that permits the expression of the protein CmWIP1 of sequence SEQ ID No. 12 in dicotyledonous plants.

Said functional regulatory polynucleotide (PG) thus makes it possible, when it is introduced artificially in a plant, to change the sex of the flowers of such a plant, and in particular makes it possible to obtain male and female or male and hermaphroditic plants, capable of self-pollination.

The invention therefore also relates to a nucleic acid comprising a polynucleotide possessing at least 95% nucleotide identity with the nucleotide sequence starting at the nucleotide in position 1 and ending at the nucleotide in position 2999 of the sequence SEQ ID No. 10 as well as a nucleic acid of complementary sequence.

The invention also relates to a nucleic acid possessing at least 95% nucleotide identity with the nucleotide sequence starting at the nucleotide in position 1 and ending at the nucleotide in position 2999 of the sequence SEQ ID No. 10, as well as a nucleic acid of complementary sequence.

The invention further relates to a nucleic acid comprising the nucleotide sequence starting at the nucleotide in position 1 and ending at the nucleotide in position 2999 of the sequence SEQ ID No. 10 or a nucleic acid of complementary sequence.

The invention also relates to a nucleic acid consisting of the nucleotide sequence starting at the nucleotide in position 1 and ending at the nucleotide in position 2999 of the sequence SEQ ID No. 10 or a nucleic acid of complementary sequence.

The regulatory polynucleotide extending from the nucleotide in position 1 to the nucleotide in position 2999 of the sequence SEQ ID No. 10 is also referred to as the nucleic acid of sequence SEQ ID No. 13 in the present description.

The invention also relates to a nucleic acid comprising at least 12 consecutive nucleotides of a regulatory polynucleotide, as defined above.

Said nucleic acid can be used as oligonucleotide probe or primer for detecting the presence of at least one copy of the allele (G) of the gene (G/g) in a sample, for amplifying a specified target sequence within the gene (G/g). Said nucleic acid can also be used for finding functional allelic variants of the gene (G/g), or can be used in a method of selection of plants possessing a specified sexual type.

Methods of detection employing nucleic acids as described above are described in the section headed “Methods of selection according to the invention”.

Said nucleic acid can also be used for inhibiting a specified target sequence within the gene (G/g) by an antisense or co-suppression approach, or by the use of double-stranded RNA (Wassenegger et al. 1996; Kooter et al. 1999) for interference.

### Nucleic Acids Comprising a Non-Functional Regulatory Polynucleotide Pg

A non-functional regulatory polynucleotide (Pa) or promoter according to the invention is a nucleic acid which:
(i) does not permit the expression of the protein CmWIP1 of sequence SEQ ID No. 12 in a host cell, or
(ii) permits the expression of this protein at a very low level in comparison with the level observed with the regulatory polynucleotide (PG), or
(iii) permits the expression of the protein CmWIP1 during the life of the plant, for a shorter time, in comparison with that observed with the regulatory polynucleotide (PG).

Said non-functional regulatory polynucleotide (Pg) thus makes it possible, when it is introduced artificially in a plant, for example replacing a polynucleotide (G), to change the sex of the flowers of such a plant, and in particular makes it possible to obtain hermaphroditic plants, capable of self-pollination, or else female plants.

Said nucleic acid can be used as oligonucleotide probe or primer for detecting the presence of at least one copy of the allele (a) of the gene (G/g) in a sample, or for amplifying a specified target sequence within the gene (G/g).

The invention relates finally to nucleic acids comprising a combination of one or more nucleic acids as defined above, for example a nucleic acid coding for a functional protein CmWIP1 under the control of a promoter of type (PG) or (Pg).

### General Definitions

According to the invention, any conventional technique of molecular biology, of microbiology and of recombinant DNA known by a person skilled in the art can be used. Such techniques are described for example by SAMBROOK et al. (1989), GLOVER (1985), GAIT (1984), HAMES and HIGGINS (1984), BERBAL (1984) and AUSUBEL et al. (1994).

Preferably, any nucleic acid and any polypeptide according to the invention is in an isolated or purified form.

The term “isolated” in the sense of the present invention denotes a biological material that has been removed from its original environment (the environment in which it is located naturally). For example, a polynucleotide present in the natural state in a plant is not isolated. The same polynucleotide separated from the adjacent nucleic acids in which it is naturally inserted in the genome of the plant is isolated. A polynucleotide of this kind can be included in a vector and/or a polynucleotide of this kind can be included in a composition and nevertheless remain in the isolated state because the vector or the composition does not constitute its natural environment.

The term “purified” does not require that the material is present in an absolutely pure form, excluding the presence of other compounds. Rather it is a relative definition.

A polynucleotide or a polypeptide is in the purified state after purification of the starting material or of the natural material of at least one order of magnitude, preferably 2 or 3 and preferably four or five orders of magnitude.

For the purposes of the present description, the expression “nucleotide sequence” can be used for denoting indiscriminately a polynucleotide or a nucleic acid. The expression “nucleotide sequence” includes the genetic material itself and therefore is not restricted to the information concerning its sequence.

The terms “nucleic acid”, “polynucleotide”, “oligonucleotide” or “nucleotide sequence” include sequences of RNA, of DNA, of cDNA or RNA/DNA hybrid sequences of more than one nucleotide, whether in the single-stranded form or in the duplex form.

The term “nucleotide” denotes the natural nucleotides (A, T, G, C) as well as modified nucleotides that comprise at least one modification such as (i) an analogue of a purine, (ii) an analogue of a pyrimidine, or (iii) a sugar analogue, said modified nucleotides being described for example in PCT application No. WO 95/04064.

For the purposes of the present invention, a first polynucleotide is regarded as being “complementary” to a second polynucleotide when each base of the first nucleotide is paired with the complementary base of the second polynucleotide, whose orientation is reversed. The complementary bases are A and T (or A and U), and C and G.

According to the invention, a first nucleic acid having at least 95% identity with a second reference nucleic acid will possess at least 95%, preferably at least 96%, 97%, 98%, 98.5%, 99%, 99.1%, 99.2%, 99.3%, 99.4%, 99.5%, 99.6%, 99.7%, 99.8% or 99.9% nucleotide identity with this second reference polynucleotide, the percentage identity between two sequences being determined as described below.

The “percentage identity” between two sequences of nucleic acids, in the sense of the present invention, is determined by comparing the two sequences aligned optimally, through a comparison window.

The portion of the nucleotide sequence in the comparison window can thus comprise additions or deletions (for example “gaps”) relative to the reference sequence (which does not comprise these additions or these deletions) so as to obtain optimum alignment between the two sequences.

The percentage identity is calculated by determining the number of positions at which an identical nucleic acid base is observed for the two sequences compared, then dividing the number of positions at which there is identity between the two nucleic acid bases by the total number of positions in the comparison window, then multiplying the result by one hundred to obtain the percentage nucleotide identity between the two sequences.

Optimum alignment of the sequences for the comparison can be effected by computer using known algorithms.

Most preferably, the percentage sequence identity is determined using the CLUSTAL W software (version 1.82), the parameters being fixed as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GRAPH DISTANCES=“hide”.

A nucleic acid possessing at least 95% nucleotide identity with a nucleic acid according to the invention includes the “variants” of a nucleic acid according to the invention.

“Variant” of a nucleic acid according to the invention means a nucleic acid which differs from the reference nucleic acid by one or more substitutions, additions or deletions of a nucleotide, relative to the reference nucleic acid. A variant of a nucleic acid according to the invention can be of natural origin, such as an allelic variant that exists naturally. Said variant nucleic acid can also be a non-natural nucleic acid, obtained for example by techniques of mutagenesis.

In general, the differences between the reference nucleic acid and the “variant” nucleic acid are reduced in such a way that the reference nucleic acid and the variant nucleic acid have nucleotide sequences that are very similar and, in many regions, identical. The nucleotide modifications present in a variant nucleic acid can be silent, which signifies that they do not affect the amino acid sequence that can be encoded by this variant nucleic acid.

The nucleotide modifications in the variant nucleic acid can also result in substitutions, additions or deletions of one or more amino acids in the sequence of the polypeptide that can be encoded by this variant nucleic acid.

Most preferably, a variant nucleic acid according to the invention having an open reading frame codes for a polypeptide that conserves the same function or the same biological activity as the polypeptide encoded by the reference nucleic acid.

Most preferably, a variant nucleic acid according to the invention that comprises an open reading frame codes for a polypeptide that conserves the capacity to be recognized by antibodies directed against the polypeptide encoded by the reference nucleic acid.

The nucleic acids of the genes orthologous to the protein CmWIP1 included in the genome of plants, and possessing a nucleotide identity of at least 95% with a nucleic acid encoding the protein CmWIP1, form part of the “variants” of a nucleic acid encoding the protein ACS.

“Fragment” of a nucleic acid according to the invention means a nucleotide sequence of reduced length relative to the reference nucleic acid, the nucleic acid fragment possessing a nucleotide sequence identical to the nucleotide sequence of the reference nucleic acid on the part in common. Said fragments of a nucleic acid according to the invention possess at least 12, 15, 18, 20, 25, 30, 35, 40, 45, 50, 60, 70, 80, 90, 100, 150, 200, 300, 400, 500, 1000, 2000 or 3000 consecutive nucleotides of the reference nucleic acid, the maximum nucleotide length of a fragment of a nucleic acid according to the invention being of course limited by the maximum nucleotide length of the reference nucleic acid.

### Probes and Primers

The nucleic acids according to the invention, and in particular the nucleotide sequences SEQ ID No. 10 and SEQ ID No. 11, their fragments of at least 12 nucleotides, the regulatory polynucleotides (PG) and (Pg), as well as the nucleic acids of complementary sequence, can be used for detecting the presence of at least one copy of a nucleotide sequence of the gene (G/g) or of a fragment or of an allelic variant of the latter in a sample.

In particular, the above probes and primers derived from the sequence SEQ ID No. 10, and in particular derived from the regulatory polynucleotide (PG), can be used for detecting the presence of the allele (G) in a dicotyledonous plant.

The nucleotide probes and primers that hybridize, in hybridization conditions of high stringency, to a nucleic acid selected from the sequences SEQ ID No. 10 and SEQ ID No. 11, or to a regulatory polynucleotide (PG) or (Pg), also form part of the invention.

The invention therefore also relates to a nucleic acid usable as probe or primer, hybridizing specifically to a nucleic acid as defined above.

The hybridization conditions given below are employed for the hybridization of a nucleic acid, probe or primer, with a length of 20 bases.

The level and specificity of hybridization depend on various parameters, such as:

a) the purity of the preparation of nucleic acid to which the probe or primer must hybridize;

b) the composition of bases of the probe or of the primer, the base pairs G−C possessing greater thermal stability than the base pairs A−T or A−U;

c) the length of the sequence of homologous bases between the probe or primer and the nucleic acid;

d) the ionic force: the hybridization rate increases with increase in the ionic force and the incubation time;

e) the incubation temperature;

f) the concentration of the nucleic acid to which the probe or primer is to hybridize;

g) the presence of denaturing agents such as agents promoting rupture of the hydrogen bonds, such as formamide or urea, which increase the stringency of hybridization;

h) the incubation time, the hybridization rate increasing with the incubation time;

i) the presence of volume excluding agents, such as dextran or dextran sulphate, which increase the hybridization rate because they increase the effective concentrations of the probe or primer and of the nucleic acid that is to hybridize, within the preparation.

The parameters defining the conditions of stringency depend on the temperature at which 50% of the paired strands separate (Tm).

For sequences comprising more than 360 bases, Tm is defined by the relation:

Tm=81.5+0.41(% G+C)+16.6 Log(concentration of cations)−0.63(% formamide)−(600/number of bases) (SAMBROOK et al., (1989), pages 9.54-9.62).

For sequences less than 30 bases long, Tm is defined by the relation: Tm=4(G+C)+2(A+T).

In suitable conditions of stringency, in which the nonspecific sequences do not hybridize, the hybridization temperature is approximately 5 to 30° C., preferably 5 to 10° C. below Tm.

“Hybridization conditions of high stringency” means, according to the invention, hybridization conditions such that the hybridization temperature is 5° C. below Tm.

The hybridization conditions described above can be adapted depending on the length and composition of bases of the nucleic acid whose hybridization is required or on the type of labelling selected, according to the techniques known by a person skilled in the art.

Suitable hybridization conditions can for example be adapted according to the teaching given in the work of NAMES and HIGGINS (1985) or in the work of AUSUBEL et al. (1989).

For purposes of illustration, the hybridization conditions used for a nucleic acid with a length of 200 bases are as follows:

### Prehybridization:

same conditions as for hybridization

duration: overnight.

### Hybridization:

5×SSPE (0.9 M NaCl, 50 mM sodium phosphate pH 7.7, 5 mM EDTA)

5× Denhardt's (0.2% PVP, 0.2% Ficoll, 0.2% SAB)

100 μg/ml salmon sperm DNA

0.1% SDS

duration: overnight.

### Washings:

2×SSC, 0.1% SDS 10 min 65° C.

1×SSC, 0.1% SDS 10 min 65° C.

0.5×SSC, 0.1% SDS 10 min 65° C.

0.1×SSC, 0.1% SDS 10 min 65° C.

The nucleotide probes or primers according to the invention comprise at least 12 consecutive nucleotides of a nucleic acid according to the invention, in particular of a nucleic acid of sequences SEQ ID No. 10 or SEQ ID No. 11 or of its complementary sequence, of a nucleic acid having 95% nucleotide identity with a sequence selected from the sequences SEQ ID No. 10 or SEQ ID No. 11 or of its complementary sequence or of a nucleic acid hybridizing in hybridization conditions of high stringency to a sequence selected from the sequences SEQ ID No. 10 or SEQ ID No. 11 or from its complementary sequence.

Preferably, nucleotide probes or primers according to the invention will have a length of at least 12, 15, 18, 20, 25, 30, 35, 40, 45, 50, 60, 100, 150, 200, 300, 400, 500, 1000, 2000 or 3000 consecutive nucleotides of a nucleic acid according to the invention.

Alternatively, a nucleotide probe or primer according to the invention will consist of and/or comprise fragments of a length of 12, 15, 18, 20, 25, 30, 35, 40, 45, 50, 60, 100, 150, 200, 300, 400, 500, 1000, 2000 or 3000 consecutive nucleotides of a nucleic acid according to the invention.

A nucleotide primer or probe according to the invention can be prepared by any suitable method well known by a person skilled in the art, including by cloning and the action of restriction enzymes or by direct chemical synthesis according to techniques such as the phosphodiester method of NARANG et al. (1979) or of BROWN et al. (1979), the method with diethylphosphoroamidites of BEAUCAGE et al. (1980) or the technique on a solid support described in European patent No. EP 0 707 592. Each of the nucleic acids according to the invention, including the oligonucleotide probes and primers described above, can be labelled, if desired, by incorporating a detectable molecule, i.e. a marker that is detectable by spectroscopic, photochemical, biochemical, immunochemical or chemical means.

For example, such markers can consist of radioactive isotopes (32P, 3H, 35S), fluorescent molecules (5-bromodeoxyuridine, fluorescein, acetylaminofluorene) or ligands such as biotin.

Preferably, the probes are labelled by incorporation of labelled molecules within the polynucleotides by primer extension, or by adding on the 5′ or 3′ ends.

Examples of non-radioactive labelling of fragments of nucleic acids are notably described in French patent No. FR 78 10 975 or in the articles of URDEA et al. (1988) or SANCHEZ PESCADOR et al. (1988).

Advantageously, the probes according to the invention can have structural characteristics of a nature that permits signal amplification, such as the probes described by URDEA et al. (1991) or in European patent No. EP 0 225 807 (Chiron).

The oligonucleotide probes according to the invention can be used notably in hybridizations of the Southern type to any nucleic acid coding for the protein CmWIP1, in particular the nucleic acids of sequences SEQ ID No. 10 or SEQ ID No. 11, or in hybridizations to RNA when looking for the expression of the corresponding transcript in a sample.

The probes according to the invention can also be used for detecting PCR amplification products or for detecting mispairings.

Nucleotide probes or primers according to the invention can be immobilized on a solid support. Said solid supports are well known by a person skilled in the art and comprise surfaces of the wells of microtitration plates, polystyrene beads, magnetic beads, strips of nitrocellulose or microparticles such as latex particles.

Accordingly, the invention further relates to a nucleic acid usable as nucleotide probe or primer, characterized in that it comprises at least 12 consecutive nucleotides of a nucleic acid as defined above, in particular of a nucleic acid of nucleotide sequences SEQ ID No. 10 and SEQ ID No. 11.

The invention also relates to a nucleic acid usable as nucleotide probe or primer, characterized in that it consists of a polynucleotide of at least 12 consecutive nucleotides of a nucleic acid according to the invention, most preferably of a nucleic acid of sequences selected from the nucleotide sequences SEQ ID No. 10 and SEQ ID No. 11.

As described above, said nucleic acid can further be characterized in that it is labelled with a detectable molecule.

A nucleic acid usable as nucleotide probe or primer for the detection or amplification of a genomic sequence, of the mRNA or of the cDNA of the gene (G/g) can further be characterized in that it is selected from the following sequences:

a) the nucleotide sequences hybridizing, in hybridization conditions of high stringency, to a nucleic acid of sequence SEQ ID No. 10 or SEQ ID No. 11; and

b) the sequences comprising at least 12 consecutive nucleotides of a nucleic acid of sequence SEQ ID No. 10 or SEQ ID No. 11.

### Vectors, Cells and Plants According to the Invention

In the combination of two genetic elements for controlling the development of the floral type of a dicotyledonous plant according to the invention, to enable at least one of the genetic control elements to be introduced artificially into the dicotyledonous plant, the nucleic acids and the regulatory polynucleotides defined above must be introduced into vectors, and then into cells.

Thus, the invention also relates to vectors, cells and transformed plants, which comprise the regulatory polynucleotides (PG) and (Pg), the nucleic acids coding for active or inactive proteins CmWIP1, as well as the nucleic acids corresponding to the alleles (G) and (g) as described above, and the primers defined above.

### Vectors

A nucleic acid as defined above, hereinafter called nucleic acid of interest, can be inserted into a suitable vector.

“Vector” in the sense of the present invention means a circular or linear molecule of DNA or of RNA which is indiscriminately of single-stranded or double-stranded form.

A recombinant vector according to the invention is preferably an expression vector, or more specifically an insertion vector, a transformation vector or an integration vector.

It can notably be a vector of bacterial or viral origin.

In all cases, the nucleic acid of interest is placed under the control of one or more sequences containing signals for regulation of its expression in the plant in question, the regulatory signals either all being contained in the nucleic acid of interest, as is the case in the constructions of nucleic acids described in the preceding section, or one or more of them, or all the regulatory signals being contained in the receiving vector into which the nucleic acid of interest was inserted.

A recombinant vector according to the invention advantageously comprises suitable transcription start and stop sequences.

Moreover, the recombinant vectors according to the invention can include one or more functional replication origins in the host cells in which their expression is required, plus, if necessary, selection marker nucleotide sequences.

The recombinant vectors according to the invention can also include one or more of the expression regulating signals as defined above in the description.

The bacterial vectors that are preferred according to the invention are for example the vectors pBR322 (ATCC No. 37 017) or the vectors such as pAA223-3 (Pharmacia, Uppsala, Sweden) and pGEM1 (Promega Biotech, Madison, Wis., United States).

We may also mention other vectors available commercially such as the vectors pQE70, pQE60, pQE9 (Qiagen), psiX174, pBluescript SA, pNH8A, pMH16A, pMH18A, pMH46A, pWLNEO, pSV2CAT, pOG44, pXTI and pSG (Stratagene).

They may also be vectors of the Baculovirus type such as the vector pVL1392/1393 (Pharmingen) used for transfecting the cells of the line Sf9 (ATCC No. CRL 1711) derived from Spodoptera frugiperda.

Preferably, and for the main application of the vectors of the invention consisting of obtaining stable, and preferably inducible, expression of a sequence coding for a protein CmWIP1 in a plant, we shall have recourse to vectors specially adapted for expression of sequences of interest in plant cells, such as the following vectors:
vector pBIN19 (BEVAN et al.), marketed by the company CLONTECH (Palo Alto, Calif., USA);
vector pBI 101 (JEFFERSON, 1987), marketed by the company CLONTECH;
vector pBI121 (JEFFERSON, 1987), marketed by the company CLONTECH;
vector pEGFP; Yang et al. (1996), marketed by the company CLONTECH;
vector pCAMBIA 1302 (HAJDUKIEWICZ et al., 1994)
intermediate and superbinary vectors derived from the vectors pSB12 and pSB1 described by Japan Tobacco (EP 672 752 and Ishida et al., 1996).

### Cells

The methods that are most widely used for introducing nucleic acids into bacterial cells can be used within the scope of this invention. This can be the fusion of receiving cells with bacterial protoplasts containing DNA, electroporation, bombardment with projectiles, infection by viral vectors, etc. Bacterial cells are often used for amplifying the number of plasmids containing the construct comprising the nucleotide sequence according to the invention. The bacteria are placed in culture and the plasmids are then isolated by methods that are well known by a person skilled in the art (see the manuals of protocols already mentioned), including the kits for purification of plasmids sold commercially, for example EasyPrepI from Pharmacia Biotech or QIAexpress Expression System from Qiagen. The plasmids thus isolated and purified are then manipulated to produce other plasmids which will be used for transfecting the plant cells.

To permit expression of a nucleic acid of interest according to the invention placed under the control of a suitable regulatory sequence, the nucleic acids or the recombinant vectors defined in the present description must be introduced into a host cell. The introduction of the polynucleotides according to the invention into a host cell can be performed in vitro, according to the techniques that are well known by a person skilled in the art.

The invention further relates to a host cell transformed by a nucleic acid according to the invention or by a recombinant vector as defined above.

Said transformed host cell is preferably of bacterial, fungal or vegetable origin.

Thus, it is notably possible to use bacterial cells of various strains of Escherichia coli or of Agrobacterium tumefaciens.

Advantageously, the transformed host cell is a plant cell or a plant protoplast.

Among the cells that can be transformed according to the method of the invention, we may mention, as examples, cells of dicotyledonous plants, preferably belonging to the Cucurbitaceae family, whose members are detailed below in the section headed “plants according to the invention”.

The hybrid plants obtained by the crossing of plants according to the invention also form part of the invention.

Preferably, it is a cell or a protoplast of a plant belonging to the species Cucumis melo.

The invention also relates to the use of a nucleic acid of interest, for making a transformed plant whose sexual phenotype is modified.

The invention also relates to the use of a recombinant vector as defined in the present description for making a transformed plant whose sexual phenotype is modified.

The invention also relates to the use of a cellular host transformed by a nucleic acid of interest, for making a transformed plant whose sexual phenotype is modified.

The invention also relates to a transformed plant comprising a plurality of host cells as defined above.

### Transformed Plants According to the Invention

The invention also relates to a transformed multicellular vegetable organism, characterized in that it comprises a host cell transformed or a plurality of host cells transformed by at least one of the nucleic acids as defined above, or by a recombinant vector comprising such a nucleic acid.

The transformed plant can contain a plurality of copies of a nucleic acid coding for the protein CmWIP1, in situations in which overexpression of the protein CmWIP1 is required. Overexpression of the protein CmWIP1 is notably required when we wish to obtain plants producing male and female flowers or else plants producing male and hermaphroditic flowers.

A plant overexpressing the protein CmWIP1 produces male and female flowers in the embodiments of the combination of two genetic elements of the invention in which the protein ACS is also overexpressed (allele A present in the homozygous or heterozygous state).

A plant overexpressing the protein CmWIP1 produces male and hermaphroditic flowers in the embodiments of the combination of two genetic elements of the invention in which there is absence of expression of an active protein ACS.

The invention therefore also relates to a transformed plant as defined above whose flowers are male and female and to a transformed plant as defined above whose flowers are male and hermaphroditic.

The transformed plant can contain a plurality of copies of a nucleic acid coding for the protein ACS, in situations in which overexpression of the protein ACS is required. Overexpression of the protein ACS is notably required when we wish to obtain plants producing female flowers, not capable of self-pollination.

A plant overexpressing the protein ACS produces male and female flowers in the embodiments of the combination of two genetic elements of the invention in which the protein CmWIP1 is also overexpressed (allele G present in the homozygous state).

A plant overexpressing the protein ACS produces female flowers in the embodiments of the combination of two genetic elements of the invention in which there is absence of expression of an active protein CmWIP1.

The invention therefore also relates to a transformed plant as defined above whose flowers are male and female and to a transformed plant as defined above whose flowers are exclusively female.

The transformed plants according to the invention all comprise at least the second genetic element of the combination according to the invention, selected from the nucleic acids, and the regulatory polynucleotides defined above in a form artificially introduced into their genome.

The hybrid plants obtained by the crossing of transformed plants according to the invention also form part of the invention.

The invention also relates to any part of a transformed plant as defined in the present description, such as the root, but also the aerial parts such as the stem, the leaf, the flower and especially the seed or the fruit.

The invention further relates to a plant seed produced by a transformed plant as defined above.

Typically, said transformed seed comprises one or more cells comprising in their genome one or more copies of the first and second genetic control elements as defined above, artificially introduced into said dicotyledonous plant permitting the synthesis of the protein CmWIP1 at a high level or at a low level, as required, in a controlled and inducible manner.

According to a preferred embodiment of a transformed plant according to the invention, the aim is to express, in a controlled manner, the protein CmWIP1, which implies that the transformed plant does not contain, as functional copy of a polynucleotide coding for the protein CmWIP1, only the copy or copies that were introduced artificially into their cells, and preferably into their genome, whereas the sequences of the gene (G/g) coding for CmWIP1, occurring naturally in the wild plant, bear at least one mutation causing a defect in expression of the gene (G/g).

The transformed plants according to the invention are dicotyledons, preferably belonging to the Cucurbitaceae family, and in particular to the genera selected from: Abobra, Acanthosicyos, Actinostemma, Alsomitra, Ampelosicyos, Anacaona, Apatzingania, Apodanthera, Bambekea, Benincasa, Biswarea, Bolbostemma, Brandegea, Bryonia, Calycophysum, Cayaponia, Cephalopentandra, Ceratosanthes, Chalema, Cionosicyos, Citrullus, Coccinia, Cogniauxia, Corallocarpus, Cremastopus, Ctenolepis, Cucumella, Cucumeropsis, Cucumis, Cucurbita, Cucurbitella, Cyclanthera, Cyclantheropsis, Dactyliandra, Dendrosicyos, Dicoelospermum, Dieterlea, Diplocyclos, Doyerea, Ecballium, Echinocystis, Echinopepon, Edgaria, Elateriopsis, Eureiandra, Fevillea, Gerrardanthus, Gomphogyne, Gurania, Guraniopsis, Gymnopetalum, Gynostemma, Halosicyos, Hanburia, Helmontia, Hemsleya, Herpetospermum, Hodgsonia, Ibervillea, Indofevillea, Kedrostis, Lagenaria, Lemurosicyos, Luffa, Marah, Melancium, Melothria, Melothrianthus, Microsechium, Momordica, Muellerargia, Mukia, Myrmecosicyos, Neoalsomitra, Nothoalsomitra, Odosicyos, Oreosyce, Parasicyos, Penelopeia, Peponium, Peponopsis, Polyclathra, Posadaea, Praecitrullus, Pseudocyclanthera, Pseudosicydium, Psiguria, Pteropepon, Pterosicyos, Raphidiocystis, Ruthalicia, Rytidostylis, Schizocarpum, Schizopepon, Sechiopsis, Sechium, Selysia, Seyrigia, Sicana, Sicydium, Sicyos, Sicyosperma, Siolmatra, Siraitia, Solena, Tecunumania, Telfairia, Thladiantha, Trichosanthes, Tricyclandra, Trochomeria, Trochomeriopsis, Tumamoca, Vaseyanthus, Wilbrandia, Xerosicyos, Zanonia, Zehneria, Zombitsia, or Zygosicyos.

Preferably, the transformed plants belong to the genus Cucumis, and to the species Cucumis melo.

### Methods of Detection According to the Invention

Identification of the system controlling the development of the floral type by the inventors made it possible to develop methods for detecting the sexual phenotype of the plants that are extremely simple, having the main characteristics detailed below.

The invention also relates to a method of detection of the presence of an allele (G) or (g), said method comprising the stages of:

1) contacting a nucleotide probe or a plurality of nucleotide probes as defined above with the sample to be tested; and

2) detecting any complex formed between the probe or probes and the nucleic acid present in the sample.

The above method of detection can be employed jointly with the method of detection of the presence of an allele (A) or (a) described in PCT application No. WO 2007/125264, which comprises the stages of:

1) contacting a nucleotide probe or a plurality of nucleotide probes as defined above with the sample to be tested; and

2) detecting any complex formed between the probe or probes and the nucleic acid present in the sample.

These two methods of detection make it possible to select plants whose phenotypes and genotypes are summarized in Table 1.

The detection of the complex between a nucleic acid and a probe can be performed by any technique known by a person skilled in the art, and in particular using labelled probes or primers, as described in the section “Probes and primers according to the invention”.

These methods are particularly advantageous as they avoid having to cultivate a dicotyledonous plant, in order to know its sexual phenotype. It is thus possible to perform the detection of the sexual phenotype of a very large sample of plants, at less cost.

### Methods of Selection According to the Invention

The above methods of detection can be employed in the methods of selection detailed below.

The invention also relates to a method of selection of the floral type of a plant belonging to the genus Cucurbitaceae, characterized in that it comprises the stages of:
a) determining the presence of the alleles (G) and (g), in a plant of interest belonging to the family Cucurbitaceae, for example using the nucleic acids as defined above, and
b) positively selecting the plant that possesses the allele (G) or the allele (g) in its genome.

The above method of selection can be employed jointly with the method of selection of the floral type of a plant belonging to the genus Cucurbitaceae that is described in PCT application No. WO 2007/125264, and that is characterized in that it comprises the stages of:
a) determining the presence of the alleles (A) and (a), in a plant of interest belonging to the family Cucurbitaceae, for example using the nucleic acids as defined above, or an antibody directed against the protein ACS and
b) positively selecting the plant that possesses the allele (A) or the allele (a) in its genome.

Determination of the presence of the alleles (A), (a), (G) and (g) can therefore be performed advantageously employing the above methods of detection.

A person skilled in the art can easily combine the methods of selection defined above, by referring to Table 1, showing the correspondence between genotype and phenotype, to obtain plants of solely female phenotype or hermaphroditic, for example.

### Methods of Obtaining a Transformed Plant According to the Invention

The transformed plants according to the invention can be obtained by any one of the numerous methods that now form part of the general knowledge of a person skilled in the art.

Methods of obtaining transformed plants according to the invention are described below.

### Method of Obtaining Transformed Plants by “TILLING”

“TILLING” (Targeted Induced Local Lesions IN Genomes) is a reverse genetics technique that is based on the capacity of an endonuclease to detect mispairings in a double strand of DNA and cut the DNA at the unpaired bases. This technique makes it possible to detect single mutation points generated by exposing the plants to a mutagenic chemical. The TILLING technique thus permits identification of a series of alleles of a given gene and is particularly well suited to the application of methods of high-throughput screening permitting the selection, in target genes of interest, of mutations induced by chemical mutagenesis.

The Tilling technique is well known by a person skilled in the art; it is described notably by McCallum et al. (2000, Plant Physiology, Vol. 123: 439-442).

For the needs of the present invention, the Tilling technique is particularly well suited to obtaining and selecting plants in which the allele (g) of the second genetic control element (G/g) has been introduced artificially.

According to the invention, the Tilling technique can also be used successfully for obtaining and selecting plants in which the allele (a) of the first genetic element (A/a) has been introduced artificially.

By definition, in view of the foregoing, the Tilling technique is suitable for obtaining and selecting plants in which both the allele (g) of the second genetic control element (G/g) and the allele (a) of the first genetic element (A/a) have been introduced artificially.

Dicotyledonous plants artificially mutated in the gene encoding the protein CmWIP1 can be obtained using the Tilling technique, for example according to a method comprising the following stages:
a) generating a collection of mutant dicotyledonous plants by chemical mutagenesis;
b) selecting, from the collection of mutant plants generated in stage a), the plants possessing a mutation or more than one mutation in the gene encoding the protein CmWIP1,
c) selecting, from the mutant plants selected in stage b), the plants that express the phenotype (g).

In certain embodiments of the above method, stage a) is performed by chemical mutagenesis of the seeds of the dicotyledonous plants of interest, by exposing the seeds to a mutagenic agent, for example to ethyl methanesulphonate, for example using the method described by Koornbeef et al. (1982, Mutat Res, Vol. 93: 109-123).

Then, a collection of plants M1 is generated from the seeds previously exposed to the mutagenic agent. The plants M1 are then self-fertilized in order to generate a collection of plants M2, which is the collection of mutant plants generated in stage a) of the method.

Then, in stage b), the DNA is extracted from each plant of the plant collection generated in stage a) and amplification of the nucleic acid of the target gene, here the gene encoding the protein CmWIP1, is performed, and the presence of mutation(s) in the sequence of the target gene is investigated by comparing with the sequence of the unmutated target gene. Then the plants mutated in the sequence of the gene encoding the target gene, here the gene encoding the protein CmWIP1, are selected.

In certain embodiments of stage b), the DNA extracted from several plants M2, for example 20 plants M2, is first mixed and the detection of mutations in the sequence of the target gene is performed on the mixture (“pool”) of DNA extracted in order to reduce the number of stages of detection of mutations that must be carried out.

Then a stage of amplification of the target sequences by PCR is carried out, using suitable nucleic acid primers and the amplicons that are generated are heated, and then cooled in order to generate DNA heteroduplexes between the original DNA from a plant that is not mutated on the target nucleic acid and the original DNA of a plant mutated on the target nucleic acid.

Then, the DNA heteroduplexes are incubated in the presence of an endonuclease that cleaves at the level of the mispairings. Then the cleaved heteroduplexes are denatured and separated. Then the separated DNA strands are submitted to the stage of detection of mutation(s) proper, for example by electrophoresis or else by HPLC in denaturing conditions (DHPLC).

In certain embodiments of stage b), mutation(s) in the target gene are detected by the technique of HPLC in denaturing conditions (“DHPLC”), as described for example by McCallum et al. (2000, Plant Physiol., Vol. 123: 439-442).

Then, in stage c), the plants that express the phenotype associated with the allele (g) of the genetic element (G/g) are selected from the plants mutated on the target gene, in this case the plants mutated in the gene encoding the protein CmWIP1.

As already mentioned above, the Tilling technique can be employed, according to the present invention, for obtaining transformed plants comprising any one of the combinations of alleles (G/g) and (A/a) described in the present description.

Accordingly, in certain embodiments of the above method, it is possible to select, in stage b), the plants (i) possessing a mutation or more than one mutation in the gene encoding the protein CmWIP1 and (ii) possessing a mutation or more than one mutation in the gene encoding the protein ACS.

In these particular embodiments, the plants that express both the phenotype associated with the allele (g) of the genetic element (G/g) and the phenotype associated with the allele (a) of the genetic element (A/a) are then selected, in stage c), from the plants mutated on the two target genes, i.e. the plants mutated in the gene encoding the protein CmWIP1 and in the gene encoding the protein ACS.

The present invention also relates to dicotyledonous plants of modified floral type, in the genome of which at least one mutation has been introduced in the gene encoding the protein CmWIP1.

The present invention also relates to dicotyledonous plants of modified floral type that have been artificially mutated in the sequence of the gene encoding the protein CmWIP1, said plants expressing the phenotype associated with the allele (g) of the genetic element (G/g).

The present invention also relates to dicotyledonous plants of modified floral type, in the genome of which (i) at least one mutation in the gene encoding the protein CmWIP1 and (ii) at least one mutation in the gene encoding the protein ACS have been introduced.

The present invention also relates to dicotyledonous plants of modified floral type that have been artificially mutated (i) in the sequence of the gene encoding the protein CmWIP1 and (ii) in the sequence of the gene encoding the protein ACS, said plants expressing both (i) the phenotype associated with the allele (g) of the genetic element (G/g) and (ii) the phenotype associated with the allele (a) of the genetic element (G/g).

### Other Methods of Obtaining Transformed Plants

The invention relates firstly to a method for obtaining a transformed plant for the purpose of inserting the allele (G) in a plant not comprising this allele.

The invention thus relates to a method for obtaining a transformed plant, belonging to the family Cucurbitaceae, bearing female flowers, characterized in that it comprises the following stages:

a) transformation of at least one vegetable cell of a plant of interest not bearing the allele (G) in its genome, by a nucleotide sequence (NG); or a recombinant vector comprising said nucleic acid;

b) selection of the transformed cells obtained in stage a) that have incorporated the nucleic acid (NG) into their genome;

c) regeneration of a transformed plant from the transformed cells obtained in stage b).

This type of method is particularly useful in that it makes it possible to insert the allele (G) into the genome of a plant, which will thus have a monoecious or andromonoecious phenotype.

The invention also relates to a method of transformation of plants for the purpose of suppressing the allele (G) in a plant, or of replacing the allele (G) by an allele (g) so as to obtain a plant of hermaphroditic phenotype or a plant of female type.

The invention therefore relates to a method for obtaining a transformed plant, belonging to the family Cucurbitaceae, bearing hermaphroditic flowers or female flowers, characterized in that it comprises the following stages:

a) replacement of the allele (G) with an allele (g) in a plant,

b) selection of the transformed cells obtained in stage a) that have integrated the allele (g) in their genome,

c) regeneration of a transformed plant from the transformed cells obtained in stage b),

d) crossing of plants obtained in stage c) for obtaining one no longer bearing the allele (G).

In a first embodiment of the above method, stage a) consists of transforming a plant having the allele (G) in its genome, by a nucleic acid of the “antisense” type as defined above, and selecting the plants no longer having the allele (G).

An identical result can be obtained by employing phenomena of homologous recombination for the purpose of replacing some or all of the nucleic acid (NG) with a nucleic acid of altered structure, which does not allow a phenotype corresponding to the allele (G) to be obtained.

Said nucleic acid of altered structure can consist of a regulatory polynucleotide (Pg), or a nucleic acid coding for a protein CmWIP1 with altered sequence.

The invention thus relates to a method for obtaining a transformed plant, belonging to the family Cucurbitaceae, bearing hermaphroditic flowers, characterized in that it comprises the following stages:

a) transformation of at least one vegetable cell of a plant of interest comprising an allele (G) by a regulatory polynucleotide (Pg) or by a nucleic acid coding for an altered protein CmWIP1; or a recombinant vector comprising such a nucleic acid;

b) selection of the transformed cells obtained in stage a) that have integrated at least one copy of a regulatory polynucleotide (Pg) or a nucleic acid coding for an altered protein CmWIP1 into their genome;

c) regeneration of a transformed plant from the transformed cells obtained in stage b);

d) crossing of plants obtained in stage c) for obtaining one no longer bearing the allele (G).

This type of method is particularly useful in that it makes it possible to obtain plants no longer comprising the allele (G), and which are of hermaphroditic or gynoecious type.

The above method can be employed with the method of transformation of plants for the purpose of inserting the allele (A), which is described in PCT application No. WO 2007/125364.

This previous method for obtaining a transformed plant, belonging to the family Cucurbitaceae, bearing female flowers, is characterized in that it comprises the following stages:

a) transformation of at least one vegetable cell of a plant of interest not comprising the allele (A) in its genome, by a nucleotide sequence (NA); or a recombinant vector comprising such a nucleic acid;

b) selection of the transformed cells obtained in stage a) that have integrated the nucleic acid (NA) into their genome;

c) regeneration of a transformed plant from the transformed cells obtained in stage b).

The invention also relates to a method of transformation of plants for the purpose of replacing the allele (A) with the allele (a).

The above method can be employed with the method of transformation of plants for the purpose of inserting the allele (A) that is described in PCT application No. WO 2007/125364.

This previous method for obtaining a transformed plant, belonging to the family Cucurbitaceae, bearing hermaphroditic flowers, is characterized in that it comprises the following stages:

a) in a plant, replacement of the allele (A) with an allele (a),

b) selection of the transformed cells obtained in stage a) that have integrated the allele (a) into their genome,

c) regeneration of a transformed plant from the transformed cells obtained in stage b),

d) crossing of plants obtained in stage c) for obtaining one no longer bearing the allele (A).

The above methods can therefore be combined with one another on the basis of Table 1, so as to obtain plants that are exclusively female or exclusively hermaphroditic, the industrial importance of which has already been discussed.

To simplify the methods for obtaining a transformed plant that is exclusively female or exclusively hermaphroditic, it is possible to carry out a preceding stage in the methods defined above, during which mutations of the genes (A/a) and (G/g) present naturally in the plant are carried out, for example by random insertion of the Mutator transposon in a population of plants of wild-type phenotype, then detecting, in the mutants obtained, those among these mutants that are of genotype (aagg), for example by means of the nucleotide probes or primers described in the examples.

According to this preferred embodiment, the transformed plant according to the invention is characterized in that it possesses a genotype (aagg) and has flowers that are exclusively hermaphroditic.

In one embodiment of the methods of obtaining a transformed plant defined above, the polynucleotide (NG), when it is used, comprises an inducible activating regulatory polynucleotide (PG).

The invention therefore also relates to a method for obtaining seeds of plants, development of which produces plants bearing female flowers, comprising the following stages:

a) cultivating a plant of interest not bearing in its genome the allele (G) as defined in the present description, transformed by a nucleotide sequence (NG) comprising an inducible activating regulatory polynucleotide (PG); or by a recombinant vector comprising this nucleic acid; in the absence of an inducing signal to which the inducible activating polynucleotide is sensitive,

b) contacting the transformed plant defined in a) with the inducible activating signal to which the inducible activating polynucleotide is sensitive,

c) recovering mature seeds, whose development produces exclusively plants bearing female flowers.

In another embodiment, an inducible repressing regulatory polynucleotide (Pg) is used for replacing the regulatory polynucleotide naturally present in the plant, and for lowering the level of protein CmWIP1 at a specified time.

### Preferred Methods of Obtaining a Plant Bearing Exclusively Female Flowers

Most preferably, the invention relates to a method of obtaining a plant bearing exclusively female flowers, characterized in that it consists of:
detecting the alleles (A), (a), (G) and (g) by applying the above methods of detection, and
obtaining a plant comprising at least one copy of the allele (A) and no copy of the allele (G), employing the methods of selection or the methods of obtaining a transformed plant as defined above.

The plants obtained according to the above method bear female flowers exclusively, and are therefore particularly interesting from an industrial standpoint, since they are not capable of self-pollination. These plants can therefore be used in methods of selection for the purpose of obtaining hybrid plants.

### Preferred Methods of Obtaining a Plant Bearing Hermaphroditic Flowers Exclusively

Most preferably, the invention relates to a method of obtaining a plant bearing hermaphroditic flowers exclusively, characterized in that it consists of:
detecting the alleles (A), (a), (G) and (g) by applying the methods of detection defined above, and
obtaining a plant that has no copy of the allele (A) and no copy of the allele (G), employing the methods of selection or the methods of obtaining a transformed plant as defined above.

The plants obtained according to the above method bear hermaphroditic flowers exclusively, and are therefore particularly interesting from an industrial standpoint, since they are capable of self-pollination. These plants can therefore be used in methods for creating pure lines.

### Methods of Transformation of the Plants According to the Invention

The methods that are employed most widely for introducing nucleic acids into plant cells can be used within the scope of the present invention.

The transformation of plant cells can be performed by various methods such as, for example, the transfer of the aforementioned vectors into the plant protoplasts after incubation of the latter in a solution of polyethylene glycol in the presence of divalent cations (Ca2+), electroporation (Fromm et al. 1985), the use of a particle gun, or cytoplasmic or nuclear micro-injection (Neuhaus et al., 1987).

One of the methods of transformation of plant cells that can be used within the scope of the invention is infection of the plant cells with a bacterial cellular host comprising the vector containing the sequence of interest. The cellular host can be Agrobacterium tumefaciens (An et al. 1986), or A. rhizogenes (Guerche et al. 1987).

Preferably, transformation of the plant cells is performed by transfer of the T region of the tumour-inducing extrachromosomal circular plasmid Ti of A. tumefaciens, using a binary system (Watson et al., 1994). To do this, two vectors are constructed. In one of these vectors, the DNA-T region was removed by deletion, with the exception of the right and left edges, a marker gene being inserted between them to permit selection in the cells of plants. The other partner of the binary system is an auxiliary plasmid Ti, a modified plasmid that no longer has DNA-T but still contains the virulence genes vir necessary for transformation of the plant cell. This plasmid is maintained in Agrobacterium.

According to a preferred embodiment, the method described by Ishida et al. (1996) can be applied for the transformation of dicotyledons. According to another protocol, transformation is performed according to the method described by Finer et al. (1992) using the tungsten or gold particle gun.

The present invention is also illustrated, but is not limited, by the following examples.

### EXAMPLES

### Example 1

### Identification of the Genetic Control Element (G/g)

Cloning of the locus g based on mapping was performed by screening the elements of recombination in a population of 12 660 plants segregating for the allele g. A plant of F1 generation resulting from crossing of PI124112 (monoecious genotype A−G−)×Gynadou (gynoecious genotype A-gg) was backcrossed with Gynadou in order to generate a population of generation F2, for the purposes of analyses.

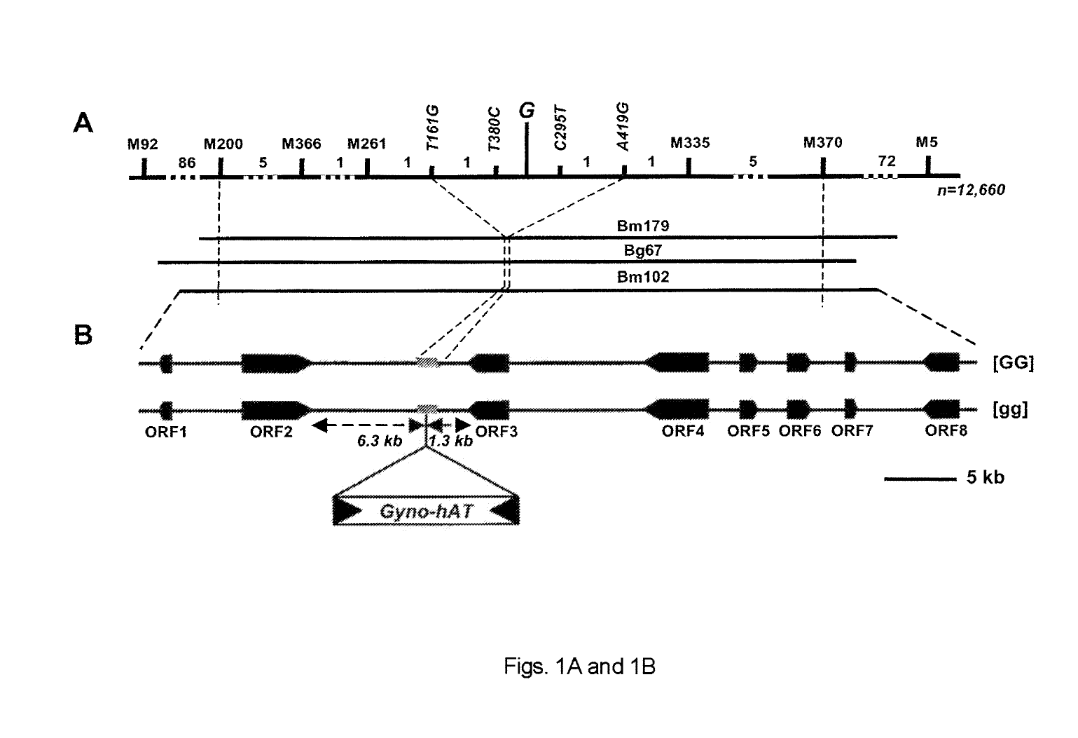

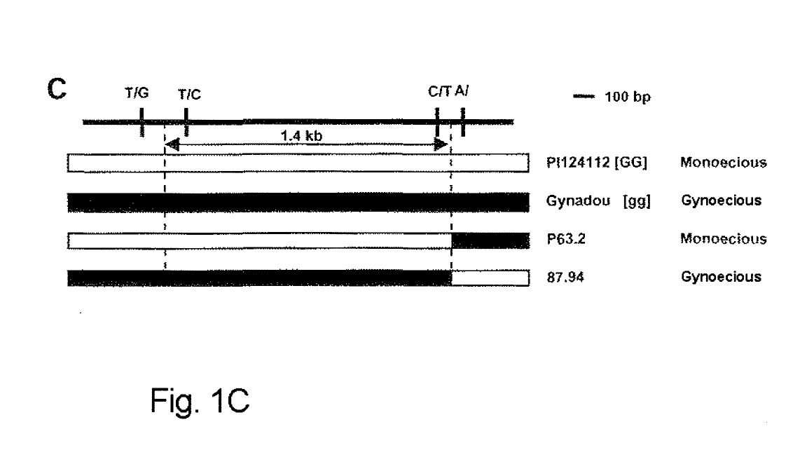

The locus g was located in an interval between markers M261 and M365 on chromosome 4 (see FIG. 1A).

The markers M261 and M365 are anchored in four different clones BAC from a genomic library of monoecious genotype, in order to generate a physical map of the locus.

An analysis of the SNPs in this interval revealed two very critical recombinants, which ultimately made it possible to reduce the locus g to a region of 1.4 kb in the monoecious BAC clone 102 (see FIG. 1C). Annotation of this clone made it possible to identify 8 open reading frames (ORFs) in this region (see FIG. 1B). No significant polymorphism was found in the 8 open reading frames surrounding this interval, when the corresponding region was sequenced in the gynoecious genotype bearing the recessive allele.

However, an insertion of 8 kb was found within the region of 1.4 kb delimited by positional cloning (FIG. 1B). This insertion corresponds to the DNA transposon of the family hAT, called Gyno-hAT (Sequence No. 14), a very widely occurring group of transposable elements (TEs).

Since this DNA transposon was found by positional cloning in the shortest interval, and since this transposon corresponds to the only significant polymorphism between the monoecious and gynoecious genotypes at this locus, this transposable element (TE) has a very strong probability of being responsible for the sex determination phenotype.

### Example 2

### Characterization of the Genetic Control Element (G/g)

Since the transposable elements (TEs) are subject to epigenetic inhibition (“silencing”) inducing suppression of transposition and of illegitimate genomic rearrangement, the methylation status of the DNA of the transposon at the locus g was examined by PCR amplification sensitive to the endonuclease McrBC.

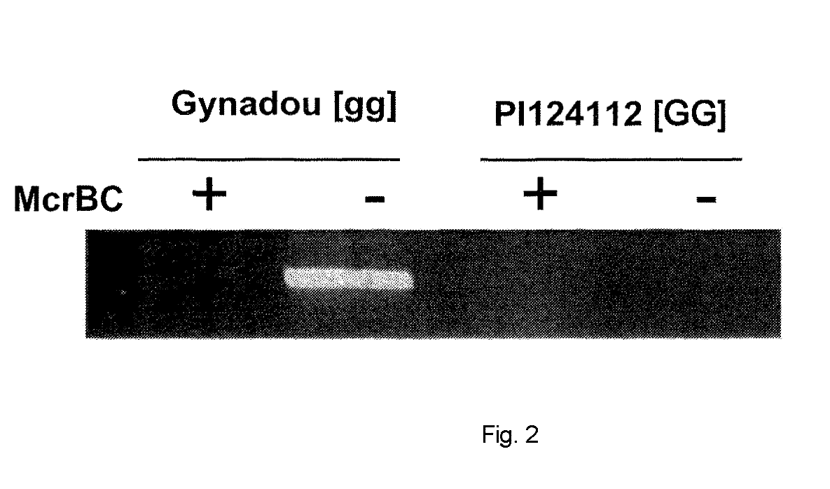

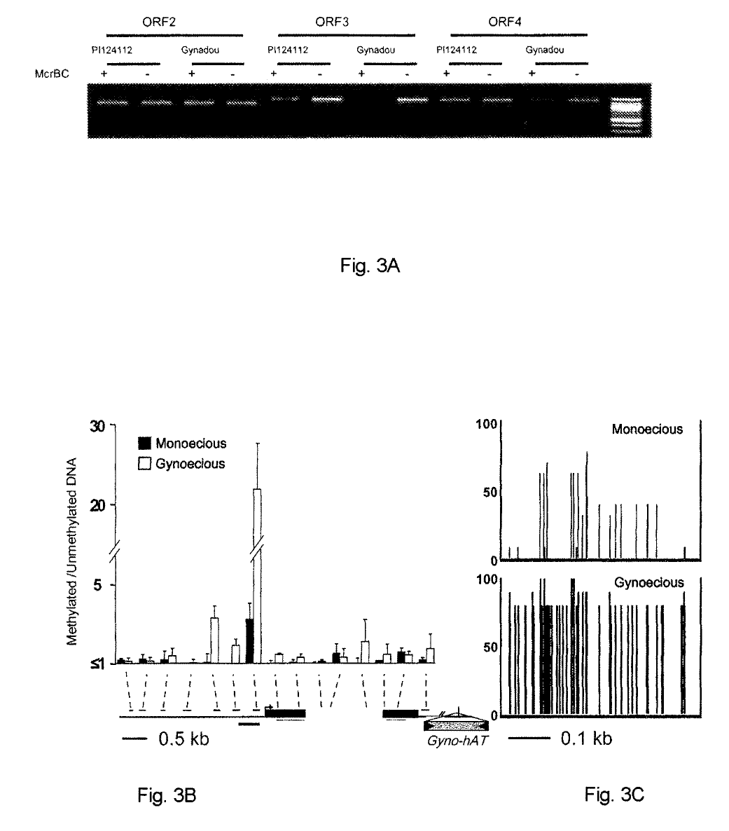

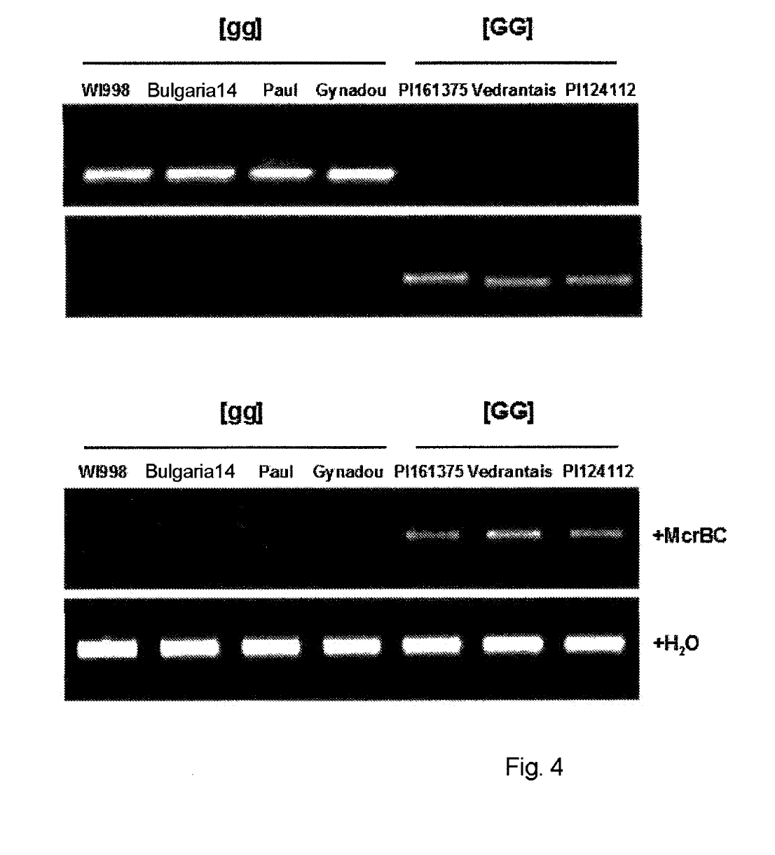

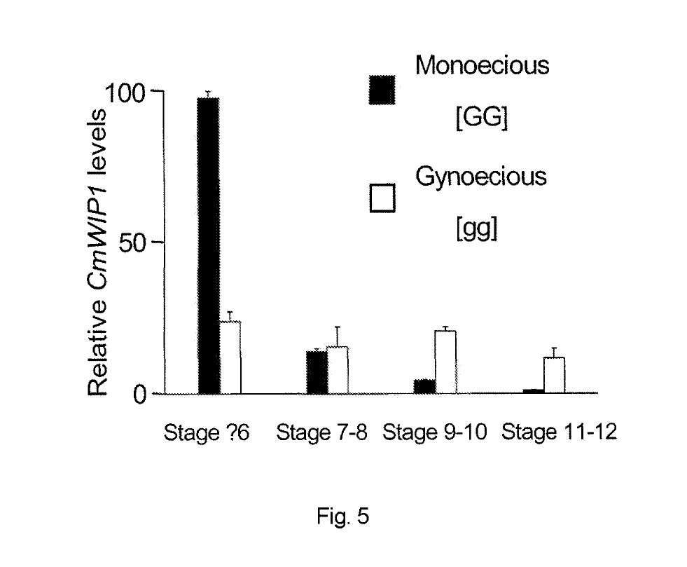

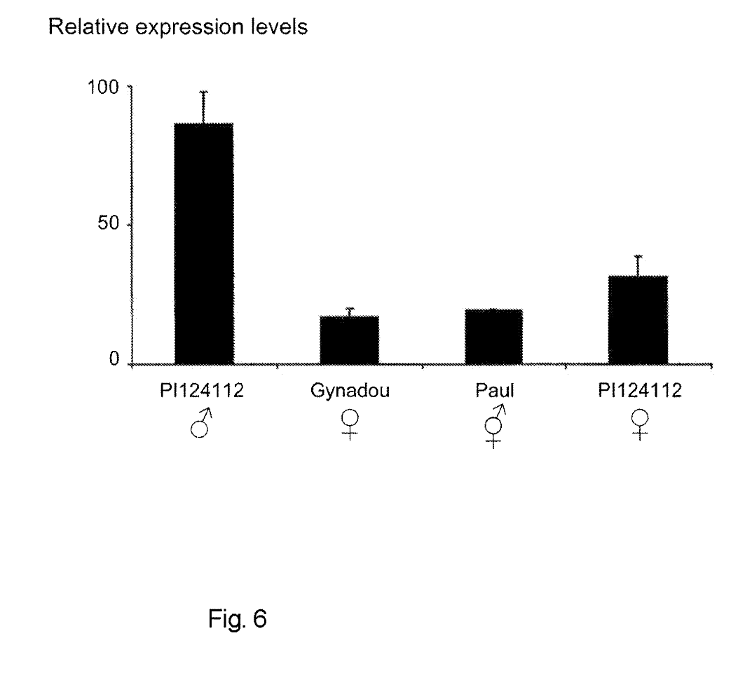

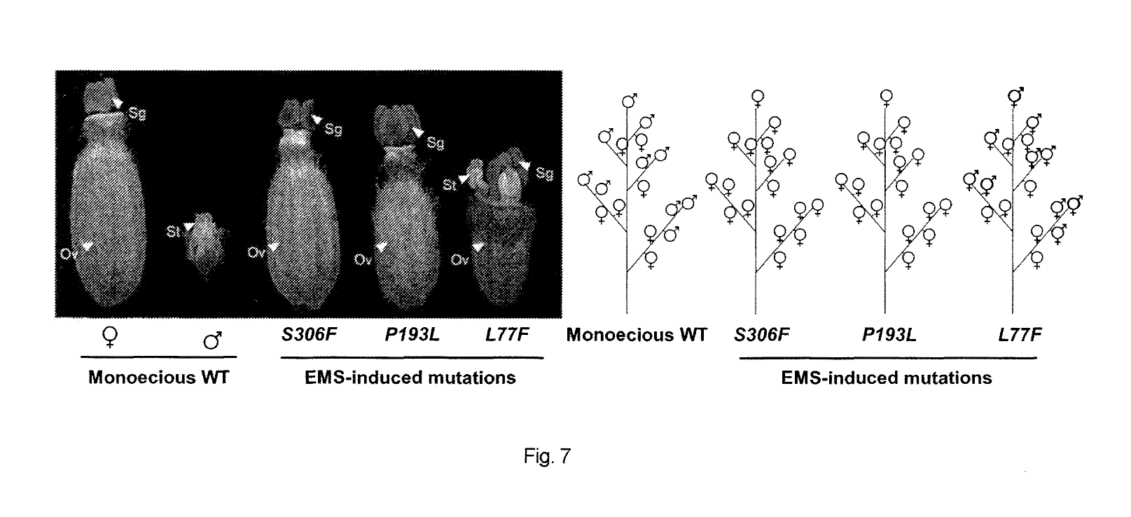

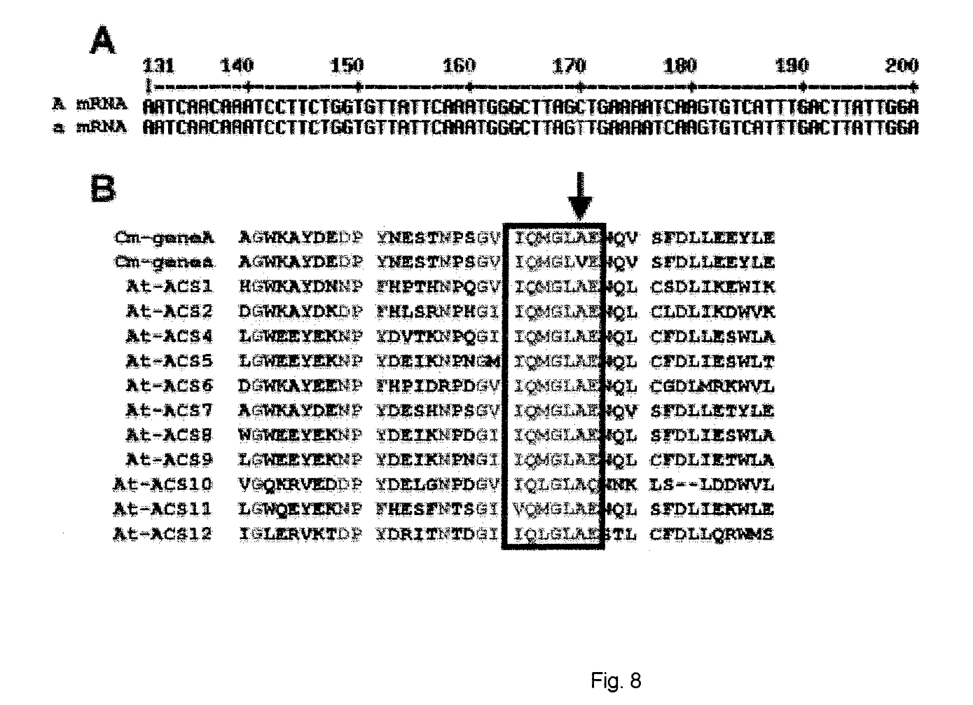

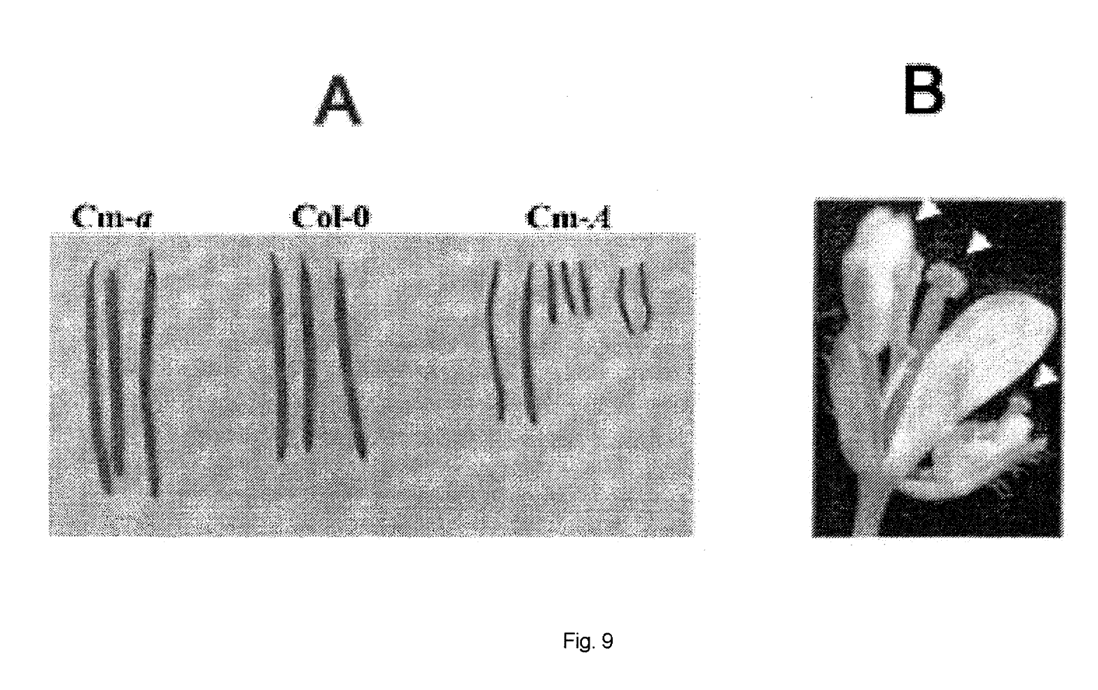

It was observed that the transposon is highly methylated (see FIG. 2).

The potential extension of DNA methylation to the genes surrounding the locus g was analysed by performing a PCR amplification sensitive to the endonuclease McrBC on the three open reading frames (ORFs) closest to the transposon.

This analysis revealed that only the open reading frame referenced ORF3, which is located at a distance of less than 1 kb from the locus g, was specifically methylated in the gynoecious genotype bearing the transposon (FIG. 3A). The open reading frame ORF3 encodes a zinc-finger transcription factor C2H2 and is homologous to the members of subfamily WIP specific to the plants (Sagasser et al., 2002).

With a view to obtaining more precise information on the profiles of methylation of the DNA of the genomic sequence CmWIP1, the whole of the gene CmWIP1 was screened by quantitative PCR sensitive to the endonuclease McrBC in the plants PI124112 (G−) and Gynadou (gg) (see FIG. 3B). The genomic DNA of Gynadou was shown to be highly methylated in the promoter region near the transcription initiation site, compared with the monoecious genotype (FIG. 3B).

The methylation of the DNA was also greater in the first exon, the intron and near the sequence 3′UTR of the gynoecious genotype, but to a lesser extent.

In order to investigate the methylation profiles of the DNA of CmWIP1, sequencing with bisulphite was performed on the highly methylated region of the promoter CmWIP1. The sequencing with bisulphite confirmed the hypermethylation phenotype of the gynoecious plants.

Of course, methylation was always greater in the promoter of the plants bearing the transposon at the locus g, and methylation was also observed in the cytosine residues, which were completely unmethylated in the monoecious genotype (see FIG. 3C).

However, interestingly, significant methylation of the DNA was observed in the promoter of PI124112.

The fact that significant methylation of DNA was detected in the promoter CmWIP1 might explain the considerable extension of inhibition of expression (“silencing”) when a transposable element (TE) was inserted nearby. Hypermethylation of CmWIP1 was confirmed in various gene pools recessive for the allele g, which reinforces the correlation between the presence of the transposon DNA and the higher state of methylation of this gene (FIG. 4).

These results strongly suggest that hypermethylation of CmWIP1, which is due to an extension of inhibition due to the transposon (“silencing”), might be the cause of sex determination in the plants bearing the allele g.

The excessive methylation of the cytosine residues in the promoter region has been linked to reduced gene expression, which could lead to a decrease in the levels of production of transcripts of CmWIP1.

### Example 3

### Profiles of Regulation of Expression of the Control Element (G/g)

In order to test the hypothesis according to which hypermethylation of the promoter region of the gene CmWIP1 might induce a decrease in expression of this gene, the profiles of regulation of the mRNA of CmWIP1 were analysed. The levels of expression of CmWIP1 were determined by quantitative PCR during sex determination and floral development.

The relative levels of transcripts were compared in the male flowers of PI124112 (G−) and in the female flowers of Gynadou (gg).

Generally, sex determination and floral development in the Cucurbitaceae have been divided into 12 stages, from initiation of the floral meristem to anthesis (Bai et al., 2004). The processes of sex determination, i.e. arrest of the inappropriate sex organ in the initial bisexual flower bud, takes place at about stages 7 and 8.

In the present case, it was shown that the gene CmWIP1 was strongly expressed in the male flower buds at stage 6, and that its expression decreases rapidly in the subsequent stages (FIG. 5). In contrast, in female flower buds, the mRNA of CmWIP1 was detected at a very low level, whatever the stage of floral development. Remarkably, the high, transient expression of CmWIP1 in male flower buds in the early stage of reproductive development coincides with arrest of development of the carpels, which was described in recent works in the Cucurbitaceae (Hao et al., 2003; Bai et al., 2004).

In order to determine the profile of spatio-temporal expression of the mRNA of CmWIP1, hybridizations of the male flowers of PI124112 (G−) and of the female flowers of Gynadou (gg) were performed in situ. The mRNA of CmWIP1 was detected early during floral development around stage 6 in the male flower buds, which is in agreement with the results of analysis by quantitative PCR.

It was found that the localization of mRNA of CmWIP1 was strongly confined in the fourth spiral of the male flower buds. This localization corresponds to the primary carpel, which will stop developing in the very next stage, in the male flowers.

The mRNA of CmWIP1 was not detectable in the female flower buds, at any stage of development. These results confirm the very low level of expression, which had already been revealed by analysis by quantitative PCR in the female flowers of Gynadou (see FIG. 7).

The levels of expression of CmWIP1 were also very low during the stages of development linked to sex determination (up to stage 6) in the hermaphroditic flower buds (aagg) and in the female flower buds of monoecious genotype (FIG. 6). In other words, expression of CmWIP1 in the early stage of floral development was still low in the flowers that will develop into a mature female structure, in all the genotypes. Taken together, these results strongly suggest that CmWIP1 has a role in sex determination in the melon, in particular in arrest of development of the carpels in the male flower buds.

### Example 4

### Determination of the Floral Type Under the Control of the Element (G/g)

For a more detailed investigation of the role of CmWIP1 in sex determination and floral development in the melon, a reverse genetics approach was developed in order to identify the function of this protein.

A strategy was adopted using the TILLING technique (Targeted Induced Local Lesions in Genomes) for screening a population of monoecious genotype (A−G−) mutagenized with ethyl methanesulphonate (EMS).

The screening of the population targeted the two exons of CmWIP1. According to alignment with the close homologues, the protein CmWIP1 is composed of two different domains, respectively a specific N-terminal domain and a conserved C-terminal domain within zinc-finger WIP proteins.

Three mutants in the coding sequence of CmWIP1 having a single base substitution were identified. The nucleotide substitutions result in the following amino acid modifications: L77F, P193L, and S306F. The substitutions of amino acids were located either in the specific N-terminal portion of CmWIP1, or in the highly conserved C-terminal domain of the protein.

The floral phenotypes were observed in the homozygous mutant plants M2, in comparison with the wild-type homozygous plants of the same family EMS.

The three mutant alleles showed redevelopment of the carpels, in comparison with the wild-type male flowers (FIG. 7).

The mutants P193L and S306F became completely gynoecious, which confirms that CmWIP1 is the gynoecium gene. The mutant L77F is a weak mutant.

The results from the examples show that the epiallele of CmWIP1 that was found in the gynoecious plants is clearly transmissible, and co-segregates with the phenotype, since genotype-phenotype relations were observed in various gene pools (see FIG. 4) and in more than twelve thousand gametes by positional cloning experiments.

Interestingly, and in agreement with the epigenetic regulation, phenotypes reverting during somatic development were occasionally observed, and correlated with demethylation of the DNA sequence of CmWIP1.

The results of the examples demonstrating the involvement of regulation of the gene CmWIP1 in floral sex determination are all the more interesting since coding genes of the zinc-finger proteins of the WIP type were found in a large variety of plants, including monocotyledons, gymnosperms and mosses. Moreover, it will be recalled that the C-terminal domains of the zinc-finger proteins of the WIP type are highly conserved.

Accordingly, control of regulation of CmWIP1, or of genes of the same family in the genomes of dicotyledons, monocotyledons, gymnosperms and mosses, at the transcriptional or post-transcriptional level during early floral development, is such as to produce controlled orientation of the development of the floral type in the plants.

### Example 5

### Spatial and Temporal Expression of the Genetic Control Element (A/a)

To investigate the expression of the genetic control element A/a in the form of the allele A, in situ hybridizations were carried out using probes specific for the allele A on plants, and more precisely on floral meristems of male, female and hermaphrodite plants, of genotype AA GG, aa GG, AA gg and aa gg. In the floral meristems A, expression is locally strong and the hybridization signal is detected specifically in the primordia of the carpels of the female and hermaphroditic flowers of the monoecious, andromonoecious, gynoecious and hermaphrodite plants. Referring to the different stages of development of the flower described for the cucumber (Bai et al., 2004), it appears that in the melon the gene (A/a) is expressed at an early stage of development of the floral meristems before a morphological distinction can be made between the male flowers and the female flowers.

In the male and hermaphroditic flowers, no expression was detected in the anthers. These data indicate that expression of the allele (A) in the carpels of the female flowers prevents development of the stamens. From the fact that, in the hermaphroditic flowers, the recessive allele (a) has the same expression profile as the allele A in the female flowers, it can be concluded that the function of gene A depends on its tissue-specificity of expression as well as on the nature of the protein ACS synthesized.

### Example 6

### Transgenesis in Arabidopsis Thaliana

The potential effects of the gene A/a, and of the protein ACS on the floral sexual phenotype and the architecture of the flower of plants not belonging to the Cucurbitaceae were investigated by transformation of Arabidopsis thaliana by Agrobacterium. The transgenic plants of Arabidopsis bearing the melon allele A or a display a phenotype at the level of the floral architecture and the siliques (FIGS. 9A and 9B). In fact the siliques of the transformants of Arabidopsis are shorter than those of a wild Arabidopsis plant and the architecture of the flowers of the transformants of Arabidopsis is very affected. These results make it possible to extend the use of the melon gene (A/a) to dicotyledonous plants not belonging to the family Cucurbitaceae.

To summarize, by cloning gene A (called CmACS-7) and gene G (called CmWIP1) it was demonstrated that expression of the protein CmWIP1 in the carpel inhibits the development of the carpel and expression of CmACS-7 or any other enzyme capable of producing ethylene in the carpel inhibits the development of the stamens. A person skilled in the art can therefore express the protein CmWIP1 in the carpel of a plant, preferably a member of the Cucurbitaceae, to block the development of the carpel or conversely inhibit the expression of the protein CmWIP1 in the carpel to promote the development of the carpel.

A person skilled in the art can also express the protein CmACS-7 in the carpel of a plant, preferably a member of the Cucurbitaceae to block the development of the stamens or conversely to inhibit expression of the protein CmACS-7 in the carpel to promote the development of the stamens. Thus, the combination of expression of the active or mutant proteins of CmACS-7 and CmWIP1 will make it possible to generate the different floral types described in Table 1.

The authors have also identified the orthologues of CmACS-7 and CmWIP1 in other species. In the cucumber (Cucumis sativa) the locus M codes for the othologue of CmACS-7.

Table of Sequences

| SEQ ID | Type | Designation |
|---|---|---|
| 1 | Nucleic acid | Genomic sequence encoding ACS |
| 2 | Nucleic acid | Genomic sequence “a” with regulatory |
| | | polynucleotide |
| 3 | Peptide | Protein ACS |
| 4 | Nucleic acid | Primer-marker M8 |
| 5 | Nucleic acid | Primer-marker M30 |
| 6 | Nucleic acid | Probe for amplicon M8/M30 |
| 7 | Nucleic acid | Probe for SEQ ID No. 1 |
| 8 | Nucleic acid | Probe for SEQ ID No. 2 |
| 9 | Nucleic acid | Vector PEC2 with gene A |
| 10 | Nucleic acid | Genomic sequence encoding CmWIP1 |
| 11 | Nucleic acid | cDNA encoding CmWIP1 |
| 12 | peptide | Protein CmWIP1 |
| 13 | Nucleic acid | Promoter of CmWIP1 |
| 14 | Nucleic acid | Gyno-hAT transposon |
| 15 | Nucleic acid | CmWIP1 gene and Gyno-hAT transposon |
| | | inserted alongside |
| 16 | Peptide | Functional protein homologous to |
| | | CmWIP1 |

### REFERENCES

An et al., (1986) Plant Physiol. 81, 86-91
Aoyama T et al., (1997) The Plant Journal, vol. 11 (3):605-612.
Ausubel et al., (1997) Current protocols in molecular biology
Beaucage et al. (1981), Tetrahedron Lett., 22:1859-1862.
Berbal, 1984.
Bevan et al., Nucleic Acids Research, vol. 12:8711-8721.
Brown et al. (1979), Methods Enzymol., 68:109-151.
Causse et al. (1995) Molecular Breeding 1: 259-272.
Christensen et al. (1996), Transgenic. Res., 5:213
Finer et al. (1992) Plant Cell Report, 11, 323-328
FROMM M. et al. (1990), Biotechnology, 8:833-839
GAIT (ed.), (1984). Nucleic Acid Hybridization.
GORLACH J, VOLRATH S, KNAUF-BEITER G, HENGY G, BECKHOVE U, KOGEL K H, OOSTENDORP M, STAUB T, WARD E, KESSMANN H, RYALS J. (1996) Benzothiadiazole, a novel class of inducers of systemic acquired resistance, activates gene expression and disease resistance in wheat. Plant Cell 8:629-43
GLOVER (ed.), 1985. DNA Cloning: A Practical Approach, Volumes I and II Oligonucleotide Synthesis, MRL Press, Ltd., Oxford, U.K.
Guerche et al. (1987), Mol Gen. Genet 206, 382
HAMES and HIGGINS, 1985. Nucleic Acid Hybridization: a practical approach, Hames & Higgins Ed. IRL Press, Oxford.
HAJDUKIEWICZ, P. SVAB. Z. AND MALIGA P. Plant Mol. Biol. 25 (6), 989-994 (1994).
Ishida et al. (1996) Nature biotechnology 14, 745-750
JEFFERSON, 1987, Plant Molecular Biology Reporter, vol. 5:387-405.
Kay et al., (1987) Science 236, 4805
Kahana, A., Silberstein, L., Kessler, N., Goldstein, R. S. and Perl-Treves, R. (2000) expression of ACC oxidase genes differs among sex genotypes and sex phases in cucumber. Plant Mol Biol. Nov; 41 (4): 517-528.
Kamachi, S., Sekimoto, H., Kondo, N. & Sakai, S., (1997). Cloning of a cDNA for a 1-aminocyclopropane-1-carboxylate synthase that is expressed during development of female flowers at the apices of Cucumis sativus L. The Plant Cell Physiol., 38:1197-206.
Kohler G and Milstein C, (1975), Nature, volume 256:495
KOOTER, J M., MATZKE, M A., AND MEYER, P. (1999) Listening to the silent genes: transgene silencing, gene regulation and pathogen control. Trends Plant Sci. 4, 430-437
Kozbor et al., (1983), Hybridoma, vol. 2 (1):7-16.
Leger O J et al., (1997), Hum Antibodies, vol. 8 (1):3-16
Martineau Pet al., (1998), J. Mol. Biol. Vol. 280(1):117-127.
Martinez, A., Sparks, C., Hart, C A., Tompson, J., and Jepson, I. (1999) Ecdysone agonist inducible transcription in transgenic tobacco plants. Plant J. 19:97-106
McNELLIS T W, 1998, The Plant Journal, vol. 14 (2): 247-257
Molina A, Hunt M D, Ryals J A (1998) Impaired fungicide activity in plants blocked in disease resistance signal transduction. Plant Cell 10:1903-14 NARANG et al. (1979), Methods Enzymol., 68: 90-98.
Neuhaus et al., (1987). Theor. Appl. Genet. 75(1), 30-36
Reinmann K A et al. (1997), Aids Res. Hum retroviruses, vol. 13 (11):933-943.
Ridder R. et al., (1995), Biotechnology (NY), vol. 13 (3):255-260.
SALTER M G et al., 1998, vol. 16 (1): 127-132
Risser, G. and Rode, J. C., 1979. Induction par le nitrate d'argent de fleurs staminées chez des plantes gynoïques de melon (Cucumis melo L.) [Silver nitrate induction of staminate flowers in gynoecious melon plants]. Annales de I'Amélioration des Plantes, 29:349-352.
Rudich, J., Halevy, A. H. and Kedar, N., 1969. Increase of femaleness of three cucurbits by treatment with Ethrel, an ethylene-releasing compound. Planta, 86:69-76.
Sambrook et al., (2001), Molecular Cloning—A laboratory manual
Sanchez Pescador, (1988), J. Clin. Microbiol., 26 (10):1934-1938.
Urdea et al. (1988), Nucleic Acids Research, 11:4937-4957.
WASSENEGGER, M., AND PELISSIER, T. (1998) A model for RNA-mediated gene silencing in higher plants Plant Mol. Biol. 37, 349-362
Watson et al. (1994) ADN recombinant [Recombinant DNA], Ed. De Boek Université, 273-292
YANG F. MOSS L G, PHILIPS G N JR. Nat. Biotechnol. 1996 Oct. 14(10):1246-51.
YANG T T, CHENG L. KAIN S R, Nucleic acids Res. 1996 Nov. 15; 24(22):4592-3.

1. Nucleic acid combination of two genetic elements for controlling the development of the floral type of a dicotyledonous plant, said combination comprising respectively:
a) a first genetic control element (A/a) present in said dicotyledonous plant, in the form of a dominant allele (A), and of a recessive allele (a), in which:
the dominant allele (A) consists of a nucleic acid (NA) permitting expression of the protein ACS (aminocyclopropane carboxylate synthase) of sequence SEQ ID No. 3,
the recessive allele (a) differs from the dominant allele by a nucleic acid (NA) non-functional in said dicotyledonous plant, and
b) a second genetic control element (G/g) present in said dicotyledonous plant, in the form of a dominant allele (G), and of a recessive allele (g), in which:
the dominant allele (G) consists of a nucleic acid (NG) permitting expression of the protein CmWIPI (“C. melo Zinc Finger Protein”) of sequence SEQ ID No. 12,
the recessive allele (g) differs from the dominant allele by a nucleic acid (NG) that is non-functional in said dicotyledonous plant, it being understood that at least the second genetic control element was introduced artificially into said dicotyledonous plant.

2. The combination according to claim 1, wherein for the first genetic control element (A/a), the respective characteristics of the dominant allele (A) and of the recessive allele (a) are as follows:
the dominant allele (A) consists of a nucleic acid (NA) comprising:
(i) a regulatory polynucleotide (PA) that is functional in a dicotyledonous plant, and
(ii) a nucleic acid whose expression is regulated by the regulatory polynucleotide (PA), said nucleic acid coding for the protein ACS (aminocyclopropane carboxylate synthase) of sequence SEQ ID No. 3, and
the recessive allele (a) differs from the dominant allele (A) by:
(i) a nucleic acid (NA) not present in the plant, or
(ii) a regulatory polynucleotide (Pa) non-functional in a dicotyledonous plant, or
(iii) a nucleic acid (Na) non-functional for the expression of an active protein ACS.

3. The combination according to claim 1, wherein the nucleic acid coding for the protein ACS comprises, from the 5′ end to the 3′ end, at least:
(i) a sequence having at least 95% identity with the polynucleotide from nucleotide 5907 to nucleotide 6086 of the sequence SEQ ID No. 1,
(ii) a sequence having at least 95% identity with the polynucleotide from nucleotide 6181 to nucleotide 6467 of the sequence SEQ ID No. 1,
and
(iii) a sequence having at least 95% identity with the polynucleotide from nucleotide 7046 to nucleotide 7915 of the sequence SEQ ID No. 1.

4. The combination according to claim 2, wherein:
(a) the regulatory polynucleotide (PA) comprises a nucleotide sequence from nucleotide 1 to nucleotide 5906 of the sequence SEQ ID No. 1; or
(b) the regulatory polynucleotide (Pa) comprises a nucleotide sequence from nucleotide 1 to nucleotide 3650 of the sequence SEQ ID No. 2; or
(c) the regulatory polynucleotide (PA), the regulatory polynucleotide (Pa), or both are sensitive to the action of an inducing signal;
(d) the regulatory polynucleotide (PA) is an inducible activating polynucleotide of transcription or translation; or
(e) the regulatory polynucleotide (Pa) is an inducible repressor polynucleotide of transcription or translation.

5.-8. (canceled)

9. The combination according to claim 1, wherein for the second genetic control element (G/g), the respective characteristics of the dominant allele (G) and of the recessive allele (g) are as follows:
(a) the dominant allele (G) consists of a nucleic acid (NG) comprising:
(i) a regulatory polynucleotide (PG) that is functional in a dicotyledonous plant, and
(ii) a nucleic acid, expression of which is regulated by the regulatory polynucleotide (PG), said nucleic acid coding for the protein CmWIP1 (“C. melo Zinc Finger Protein”) of sequence SEQ ID No. 12, and
(b) the recessive allele (g) differs from the dominant allele (G) by:
(i) a nucleic acid (NG) not present in the plant, or
(ii) a regulatory polynucleotide (Pg) that is non-functional in a dicotyledonous plant, or
(iii) a non-functional nucleic acid (Ng) for the expression of an active protein CmWIP1.

10. The combination according to claim 9, wherein the nucleic acid coding for the protein CmWIPI comprises, from the 5′ end to the 3′ end, at least:
(i) one sequence having at least 95% identity with the polynucleotide from nucleotide 3000 to nucleotide 3617 of the sequence SEQ ID No. 10, and
(ii) one sequence having at least 95% identity with the polynucleotide from nucleotide 5458 to nucleotide 5901 of the sequence SEQ ID No. 10.

11. The combination according to claim 9, wherein,
(a) the regulatory polynucleotide (PG) comprises the nucleotide sequence SEQ ID No. 11; or
(b) the regulatory polynucleotide (PG), the regulatory polynucleotide (Pg) or both are sensitive to the action of an inducing signal; or
(c) the regulatory polynucleotide (PG) is an inducible activating polynucleotide of transcription or translation; or
(d) the regulatory polynucleotide (Pg) is an inducible repressor polynucleotide of transcription or translation.

12-14. (canceled)

15. Nucleic acid comprising, from the 5′ end to the 3′ end, at least:
(i) one sequence having at least 98.5% identity with the polynucleotide from nucleotide 3000 to nucleotide 3617 of the sequence SEQ ID No. 10, and
(ii) a sequence having at least 99.5% identity with the polynucleotide from nucleotide 5458 to nucleotide 5901 of the sequence SEQ ID No. 10.

16. (canceled)

17. The nucleic acid of claim 15 comprising a nucleotide sequence:
(a) extending from nucleotide 3000 to nucleotide 5901 of the sequence SEQ ID No. 10; or
(b) having the sequence of SEQ ID no. 10.

18. Nucleic acid comprising a nucleotide sequence bearing at least one alteration selected from a mutation, an insertion or a deletion, relative to the nucleic acid from nucleotide 1 to nucleotide 2999 of the sequence SEQ ID No. 10, said altered nucleic acid leading to altered expression of the protein CmWIP1 of sequence SEQ ID No. 12, when it controls the expression of said protein, relative to the expression of the protein CmWIP1 controlled by the nucleic acid from nucleotide 1 to nucleotide 2999 of the sequence SEQ ID No. 10.

19. Recombinant vector comprising a nucleic acid combination of claim 1 wherein the nucleic acid comprises:
(a) a first genetic control element (A/a) and a second genetic control element (G/g) wherein:
i) the dominant allele (A) consists of a nucleic acid (NA) permitting expression of the protein ACS (aminocyclopropane carboxylate synthase) of sequence SEQ ID No. 3.
ii) the recessive allele (a) differs from the dominant allele by a nucleic acid (NA) non-functional in a dicotyledonous plant,
iii) the dominant allele (G) consists of a nucleic acid (NG) permitting expression of the protein CmWIPI of sequence SEQ ID No. 12 comprising a regulatory polynucleotide (PG) that is functional in a dicotyledonous plant, and a nucleic acid, expression of which is regulated by the regulatory polynucleotide (PG), said nucleic acid coding for the protein CmWIP1 of sequence SEQ ID No. 12; and
iv) the recessive allele (g) differs from the dominant allele by a nucleic acid (NG) that is not present in the plant, or a regulatory polynucleotide (Pg) that is non-functional in a dicotyledonous plant, or a non-functional nucleic acid (Ng) for the expression of an active protein CmWIP1; or
(b) a first genetic control element (A/a) and a second genetic control element (G/g) as in (a) and wherein the nucleic acid coding for the protein CmWIPI comprises, from the 5′ end to the 3′ end, at least one sequence having at least 95% identity with the polynucleotide from nucleotide 3000 to nucleotide 3617 of the sequence SEQ ID No. 10, and one sequence having at least 95% identity with the polynucleotide from nucleotide 5458 to nucleotide 5901 of the sequence SEQ ID No. 10; or
(c) a first genetic control element (A/a) and a second genetic control element (G/g) as in (a) or (b), wherein the regulatory polynucleotide (PG) comprises the nucleotide sequence SEQ ID No. 11; or
(d) a first genetic control element (A/a) and a second genetic control element (G/g) as in (a), (b) or (c), wherein the regulatory polynucleotide (PG) and/or regulatory polynucleotide (Pg) is(are) sensitive to the action of an inducing signal; or
e. a first genetic control element (A/a) and a second genetic control element (G/g) as in (a), (b), (c), or (d), wherein the regulatory polynucleotide (PG) is an inducible activating polynucleotide of transcription or translation; or
(f) a first genetic control element (A/a) and a second genetic control element (G/g) as in (a), (b), (c), or (d), wherein the regulatory polynucleotide (Pg) is an inducible repressor polynucleotide of transcription or translation; or
(g) from the 5′ end to the 3′ end, at least:
(i) one sequence having at least 98.5% identity with the polynucleotide from nucleotide 3000 to nucleotide 3617 of the sequence SEQ ID No. 10, and
(ii) a sequence having at least 99.5% identity with the polynucleotide from nucleotide 5458 to nucleotide 5901 of the sequence SEQ ID No. 10; or
(h) (i) a regulatory polynucleotide (PG) that is functional in a dicotyledonous plant, and
(ii) a nucleic acid, expression of which is regulated by the regulatory polynucleotide (PG), said nucleic acid coding for the protein CmWIP1 (“C. melo Zinc Finger Protein”) of sequence SEQ ID No. 12, and which has the of sequence of SEQ ID No. 10; or
(i) a nucleotide sequence extending from nucleotide 3000 to nucleotide 5901 of the sequence SEQ ID No. 10; or
(j) a nucleotide sequence bearing at least one alteration selected from a mutation, an insertion or a deletion, relative to the nucleic acid from nucleotide 1 to nucleotide 2999 of the sequence SEQ ID No. 10, said altered nucleic acid leading to altered expression of the protein CmWIP1 of sequence SEQ ID No. 12, when it controls the expression of said protein, relative to the expression of the protein CmWIP1 controlled by the nucleic acid from nucleotide 1 to nucleotide 2999 of the sequence SEQ ID No. 10.

20. Host cell transformed by a recombinant vector according to claim 19.

21. Host cell according to claim 20, characterized in that it is a cell of a plant belonging to the family Cucurbitaceae.

22. Plant belonging to the family Cucurbitaceae transformed by a recombinant vector according to claim 19.

23. Transformed plant according to claim 22, which comprises at least one allele (G) consisting of a nucleic acid (NG) permitting expression of the protein CmWIPI (“C. melo Zinc Finger Protein”) of sequence SEQ ID No. 12.

24. Transformed plant comprising a plurality of host cells according to claim 20.

25. Host cell transformed by a first and a second nucleic acid, respectively:
(1) wherein the first nucleic acid is selected from:
(1a) a nucleic acid determining an allele A or (a) as defined in claim 1,
(1b) a nucleic acid comprising, from the 5′ end to the 3′ end, at least:
(i) one sequence having at least 95% identity with the polynucleotide from nucleotide 5907 to nucleotide 6086 of the sequence SEQ ID No. 1,
(ii) one sequence having at least 95% identity with the polynucleotide from nucleotide 6181 to nucleotide 6467 of the sequence SEQ ID No. 1, and
(iii) one sequence having at least 95% identity with the polynucleotide from nucleotide 7046 to nucleotide 7915 of the sequence SEQ ID No. 1,
(1c) a nucleic acid in the form of allele (A) of sequence SEQ ID No. 1,
(1d) a nucleic acid in the form of the allele (a) of sequence SEQ ID No. 2,
(1e) a nucleic acid comprising a nucleotide sequence from nucleotide 1 to nucleotide 5906 of the sequence SEQ ID No. 1,
(1f) a nucleic acid comprising a nucleotide sequence bearing at least one alteration selected from a mutation, an insertion or a deletion, relative to the nucleic acid from nucleotide 1 to nucleotide 5907 of the sequence SEQ ID No. 1, said altered nucleic acid leading to altered expression of the protein ACS of sequence SEQ ID No. 3, when it controls the expression of said protein, relative to the expression of the protein ACS controlled by the nucleic acid from nucleotide 1 to nucleotide 5907 of the sequence SEQ ID No. 1, or
(1g) a nucleic acid comprising a sequence extending from nucleotide 1 to nucleotide 3650 of the sequence SEQ ID No. 2, and
(2) wherein the second nucleic acid is selected from:
(2a) a first genetic control element (A/a) and a second genetic control element (G/g) wherein:
i) the dominant allele (A) consists of a nucleic acid (NA) permitting expression of the protein ACS (aminocyclopropane carboxylate synthase) of sequence SEQ ID No. 3,
ii) the recessive allele (a) differs from the dominant allele by a nucleic acid (NA) non-functional in a dicotyledonous plant,
iii) the dominant allele (G) consists of a nucleic acid (NG) permitting expression of the protein CmWIPI of sequence SEQ ID No. 12 comprising a regulatory polynucleotide (PG) that is functional in a dicotyledonous plant, and a nucleic acid, expression of which is regulated by the regulatory polynucleotide (PG), said nucleic acid coding for the protein CmWIP1 of sequence SEQ ID No. 12; and
iv) the recessive allele (g) differs from the dominant allele by a nucleic acid (NG) that is not present in the plant, or a regulatory polynucleotide (Pg) that is non-functional in a dicotyledonous plant, or a non-functional nucleic acid (Ng) for the expression of an active protein CmWIP1; or
(2b) a first genetic control element (A/a) and a second genetic control element (G/g) as in (2a) and wherein the nucleic acid coding for the protein CmWIPI comprises, from the 5′ end to the 3′ end, at least one sequence having at least 95% identity with the polynucleotide from nucleotide 3000 to nucleotide 3617 of the sequence SEQ ID No. 10, and one sequence having at least 95% identity with the polynucleotide from nucleotide 5458 to nucleotide 5901 of the sequence SEQ ID No. 10; or
(2c) a first genetic control element (A/a) and a second genetic control element (G/g) as in (2a) or (2b), wherein the regulatory polynucleotide (PG) comprises the nucleotide sequence SEQ ID No. 11; or
(2d) a first genetic control element (A/a) and a second genetic control element (G/g) as in (2a), (2b) or (2c), wherein the regulatory polynucleotide (PG) and/or regulatory polynucleotide (Pg) is(are) sensitive to the action of an inducing signal; or
(2e) a first genetic control element (A/a) and a second genetic control element (G/g) as in (2a), (2b), (2c), or (2d), wherein the regulatory polynucleotide (PG) is an inducible activating polynucleotide of transcription or translation; or
(2f) a first genetic control element (A/a) and a second genetic control element (G/g) as in (2a), (2b), (2c), or (2d), wherein the regulatory polynucleotide (Pg) is an inducible repressor polynucleotide of transcription or translation; or
(2g) from the 5′ end to the 3′ end, at least:
(i) one sequence having at least 98.5% identity with the polynucleotide from nucleotide 3000 to nucleotide 3617 of the sequence SEQ ID No. 10, and
(ii) a sequence having at least 99.5% identity with the polynucleotide from nucleotide 5458 to nucleotide 5901 of the sequence SEQ ID No. 10; or
(2h) (i) a regulatory polynucleotide (PG) that is functional in a dicotyledonous plant, and
(ii) a nucleic acid, expression of which is regulated by the regulatory polynucleotide (PG), said nucleic acid coding for the protein CmWIP1 (“C. melo Zinc Finger Protein”) of sequence SEQ ID No. 12, and which has the of sequence of SEQ ID No. 10; or
(2i) a nucleotide sequence extending from nucleotide 3000 to nucleotide 5901 of the sequence SEQ ID No. 10; or
(2j) a nucleotide sequence bearing at least one alteration selected from a mutation, an insertion or a deletion, relative to the nucleic acid from nucleotide 1 to nucleotide 2999 of the sequence SEQ ID No. 10, said altered nucleic acid leading to altered expression of the protein CmWIP1 of sequence SEQ ID No. 12, when it controls the expression of said protein, relative to the expression of the protein CmWIP1 controlled by the nucleic acid from nucleotide 1 to nucleotide 2999 of the sequence SEQ ID No. 10; or
(2k) a recombinant vector comprising a nucleic acid of (2a), (2b), (2c), (2d), (2e), 12f), (2g), (2h) or (2i).

26. Host cell according to claim 25, characterized in that it is a cell of a plant belonging to the family Cucurbitaceae.

27. Transformed plant comprising a plurality of host cells according to claim 25.

28. Nucleic acid, usable as probe or primer, hybridizing specifically to a nucleic acid as defined in claim 9 wherein:
(a) the nucleic acid coding for the protein CmWIPI comprises, from the 5′ end to the 3′ end, at least:
(i) one sequence having at least 95% identity with the polynucleotide from nucleotide 3000 to nucleotide 3617 of the sequence SEQ ID No. 10, and
(ii) one sequence having at least 95% identity with the polynucleotide from nucleotide 5458 to nucleotide 5901 of the sequence SEQ ID No. 10; or
(b) the regulatory polynucleotide (PG) comprises the nucleotide sequence SEQ ID No. 11; or
(c) the regulatory polynucleotide (PG) and/or regulatory polynucleotide (Pg) is(are) sensitive to the action of an inducing signal; or
(d) the regulatory polynucleotide (PG) is an inducible activating polynucleotide of transcription or translation; or
(e) the regulatory polynucleotide (Pg) is an inducible repressor polynucleotide of transcription or translation; or
(f) the nucleic acid comprising, from the 5′ end to the 3′ end, at least:
(i) one sequence having at least 98.5% identity with the polynucleotide from nucleotide 3000 to nucleotide 3617 of the sequence SEQ ID No. 10, and
(ii) a sequence having at least 99.5% identity with the polynucleotide from nucleotide 5458 to nucleotide 5901 of the sequence SEQ ID No. 10.
(g) the nucleic acid in the form of the allele (G) of sequence SEQ ID No. 10; or
(h) the nucleic acid comprising a nucleotide sequence extending from nucleotide 3000 to nucleotide 5901 of the sequence SEQ ID No. 10; or
(i) the nucleic acid comprising a nucleotide sequence bearing at least one alteration selected from a mutation, an insertion or a deletion, relative to the nucleic acid from nucleotide 1 to nucleotide 2999 of the sequence SEQ ID No. 10, said altered nucleic acid leading to altered expression of the protein CmWIP1 of sequence SEQ ID No. 12, when it controls the expression of said protein, relative to the expression of the protein CmWIP1 controlled by the nucleic acid from nucleotide 1 to nucleotide 2999 of the sequence SEQ ID No. 10.

29. Method for detecting the presence of an allele (G) or (g), wherein the dominant allele (G) consists of a nucleic acid (NG) permitting expression of the protein CmWIPI (“C. melo Zinc Finger Protein”) of sequence SEQ ID No. 12, and wherein the recessive allele (g) differs from the dominant allele by a nucleic acid (NG) that is non-functional in said dicotyledonous plant, said method comprising the steps of:
1) contacting a nucleotide probe or a plurality of nucleotide probes according to claim 28 with the sample to be tested; and
2) detecting any complex formed between the probe or probes and the nucleic acid present in the sample.

30. Method for obtaining plants artificially mutated in the gene encoding the protein CmWIP1 comprising the following steps:
a) generating a collection of mutant dicotyledonous plants by chemical mutagenesis;
b) selecting, from the collection of mutant plants generated in step a), the plants possessing a mutation or more than one mutation in the gene encoding the protein CmWIP1; and
c) selecting, from the mutant plants selected in step b), the plants that express the phenotype (g).

31. Method according to claim 30, wherein the plants are moreover mutated in the gene encoding the protein ACS and wherein:
the plants (i) possessing a mutation or more than one mutation in the gene encoding the protein CmWIP1 and (ii) possessing a mutation or more than one mutation in the gene encoding the protein ACS are selected in step b), and
the plants that express both the phenotype associated with the allele (g) of the genetic element (G/g) and the phenotype associated with the allele (a) of the genetic element (A/a) are selected in step c) from the plants mutated in the gene encoding the protein CmWIP1 and in the gene encoding the protein ACS.

32. Dicotyledonous plant with modified floral type that has been artificially mutated:
(a) in the sequence of the gene encoding the protein GmWIP1, said plant expressing the phenotype associated with the allele (g) of the genetic element (G/g); or
(b) in the sequence of the gene encoding the protein CmWIP1 and in the sequence of the gene encoding the protein ACS, said plant expressing both the phenotype associated with the allele (g) of the genetic element (G/g) and the phenotype associated with the allele (a) of the genetic element (A/a).

33. (canceled)

34. Method for obtaining a transformed plant, belonging to the family Cucurbitaceae, characterized in that it comprises the following steps:
a) transformation of at least one vegetable cell of a plant of interest not comprising the allele (G) in its genome, by a nucleotide sequence (NG) or a recombinant vector comprising such a nucleic acid, wherein the allele (G) and the nucleotide sequence (NG) are as defined in claim 1;
b) selection of the transformed cells obtained in step a) that have integrated the nucleic acid (NG) into their genome; and
c) regeneration of a transformed plant from the transformed cells obtained in step b).

35. Method for obtaining a transformed plant, belonging to the family Cucurbitaceae, characterized in that it comprises the following steps:
a) in a plant, replacing the allele (G) by an allele (g), wherein the allele (G) and the allele (g) are as defined in claim 1,
b) selection of the transformed cells derived from a plant obtained in stage a) and that have integrated the allele (g) in their genome,
c) regeneration of a transformed plant from the transformed cells obtained in stage b), and
d) crossing of plants obtained in stage c) to obtain a plant no longer bearing the allele (G)

36. A host cell or a plant belonging to the family Cucurbitaceae transformed by a nucleic acid as defined in claim 9 wherein:
(a) a first genetic control element (A/a) and a second genetic control element (G/g) wherein:
i) the dominant allele (A) consists of a nucleic acid (NA) permitting expression of the protein ACS (aminocyclopropane carboxylate synthase) of sequence SEQ ID No. 3,
ii) the recessive allele (a) differs from the dominant allele by a nucleic acid (NA) non-functional in a dicotyledonous plant,
iii) the dominant allele (G) consists of a nucleic acid (NG) permitting expression of the protein CmWIPI of sequence SEQ ID No. 12 comprising a regulatory polynucleotide (PG) that is functional in a dicotyledonous plant, and a nucleic acid, expression of which is regulated by the regulatory polynucleotide (PG), said nucleic acid coding for the protein CmWIP1 of sequence SEQ ID No. 12; and
iv) the recessive allele (g) differs from the dominant allele by a nucleic acid (NG) that is not present in the plant, or a regulatory polynucleotide (Pg) that is non-functional in a dicotyledonous plant, or a non-functional nucleic acid (Ng) for the expression of an active protein CmWIP1; or
(b) a first genetic control element (A/a) and a second genetic control element (G/g) as in (a) and wherein the nucleic acid coding for the protein CmWIPI comprises, from the 5′ end to the 3′ end, at least one sequence having at least 95% identity with the polynucleotide from nucleotide 3000 to nucleotide 3617 of the sequence SEQ ID No. 10, and one sequence having at least 95% identity with the polynucleotide from nucleotide 5458 to nucleotide 5901 of the sequence SEQ ID No. 10; or
(c) a first genetic control element (A/a) and a second genetic control element (G/g) as in (a) or (b), wherein the regulatory polynucleotide (PG) comprises the nucleotide sequence SEQ ID No. 11; or
(d) a first genetic control element (A/a) and a second genetic control element (G/g) as in (a), (b) or (c), wherein the regulatory polynucleotide (PG) and/or regulatory polynucleotide (Pg) is(are) sensitive to the action of an inducing signal; or
(e) a first genetic control element (A/a) and a second genetic control element (G/g) as in (a), (b), (c), or (d), wherein the regulatory polynucleotide (PG) is an inducible activating polynucleotide of transcription or translation; or
(f) a first genetic control element (A/a) and a second genetic control element (G/g) as in (a), (b), (c), or (d), wherein the regulatory polynucleotide (Pg) is an inducible repressor polynucleotide of transcription or translation; or
(g) from the 5′ end to the 3′ end, at least:
(i) one sequence having at least 98.5% identity with the polynucleotide from nucleotide 3000 to nucleotide 3617 of the sequence SEQ ID No. 10, and
(ii) a sequence having at least 99.5% identity with the polynucleotide from nucleotide 5458 to nucleotide 5901 of the sequence SEQ ID No. 10; or
(h) (i) a regulatory polynucleotide (PG) that is functional in a dicotyledonous plant, and
(ii) a nucleic acid, expression of which is regulated by the regulatory polynucleotide (PG), said nucleic acid coding for the protein CmWIP1 (“C. melo Zinc Finger Protein”) of sequence SEQ ID No. 12, and which has the of sequence of SEQ ID No. 10; or
(i) a nucleotide sequence extending from nucleotide 3000 to nucleotide 5901 of the sequence SEQ ID No. 10; or
(j) a nucleotide sequence bearing at least one alteration selected from a mutation, an insertion or a deletion, relative to the nucleic acid from nucleotide 1 to nucleotide 2999 of the sequence SEQ ID No. 10, said altered nucleic acid leading to altered expression of the protein CmWIP1 of sequence SEQ ID No. 12, when it controls the expression of said protein, relative to the expression of the protein CmWIP1 controlled by the nucleic acid from nucleotide 1 to nucleotide 2999 of the sequence SEQ ID No. 10.


SEQUENCE LISTING (16 sequences)

SEQ ID NO: 1 - DNA, 13380 bp, Cucumis melo
tatacacattttgtaatgataaaattagaagactagttgattaattgtttaggctttattatatattcatcataagtcttttttgtagccatttaggtttgttttcgtcgaattaatcttataaacactatttttattcgtaaattccgttgctttcttatttactttatatcaatgctttaaaacatcaatctagtttttaaaaatcaatatatatgtttgcacacaccattattatcgtatgttactctatctattactgacaaacgttatgaaattttattatatttgtaattatcttttgcagttttgtcatttaaaatcgtttttcttaaaagaattatgttgttattttaaaattttggctaaagaatcacgtggagaattagatatatcaaacctttcatctttgagatgaaagattacatcaattactattaactaagcttactttgataaattaaaatcatattaaaacaaatagtccgtaaaagaatataattttgaaaaactaaacagtcatcaaacaacgcgtgttagcttttaatatatattatgatatgttaagtgaaaataaagttgaagtgtatgaagccaaaagagaagtcgttttcacttgttgagttctaatttctaggatggttctatgtaaagtacttcctcttccaaaattggaatccaactcactacttataaacatcatttattcgtcatcttaattacaataccaactcttatttttgtctcatctatcatcacactcactaattaacattaccattatcttatatcattttatgaactcattatttaacaaataaatcacttaaaagtttaacttcaaaaaaaaaaaggaagaaagaaagaaggtttgaaattacactatttgcaattaattatgttttatgaaaactttctaatactttaattttatgtcgaatcgtttgtcgaatcgtttctcttttatcctactacaaaaatattataaaatgattataaatggctaaaatatatagtatgtgtatttcataaatttaagaaaatgttttcgaatacagtcaaatgaactaaaatatttacaaaaatataacaaaatttcatatgtatatcgaataaaatttaaaaatttgaagactaaatttgtaatataattacatattaaagtaatttttagatgtgtgggtattatataataataatgttgggaaggtgagggcatgaggcagctggagggataaggactagggattgttttatatcctttttcacatttaatttttgatgctaattaatttgttgccaatcatttcatcacttttttttttttttttggttctaatttatttactttatatggaaaataaataaaagaaaaatgaaagaaagaaaaaagtggttttcaaatcaatagaaaaaacaaacaactccaactttaatggcttgaaaacaaatgcattctaaaattaaaccttatgattgatttgatttttattccccttttttacacttttcattttcatcataattatatcttcagttacctgtccaccaattacaccatcaaatgtggattattgggattcttttttttttttttaagattatcttacggctttcatttttttcgtatctttatgacggtttgatagacgtaaaagtggttattgtgttatagagatttgtattattttgatattatggaaggattcgtttgagtaaaattataaaaatcagaggggtgtcgtttaaaaatgtaagtaatccaacacaaaaaaataattatcataaaatgtaaaaaaaagggttagattgaaaacaaacgaaacaaatgagttttgtattataaatcgacctaaaatgttcaacccaaacatggattacgatacgaccgattcatctcattacagctcatcgatcctaaaaatgtgaagagaagtattggatataattattacttaaaaagataatagaaaaaggaaatcagcaaaattagggttctttaataagttataaaactcatttatatacaaaattaattacattacaaaaggtgggaatgtggatttagacatacaacctataataattaattaaaaacaatacacatgtttcacaatttgagataattaaattttaatccccatttgataagtaatgattttatcttataaattagtttgttaggtctatactttatttgtttatttatttattcttactcttttttaattatatttttacttatatcccaagcttcattaacgattaatctaagtttgaaatgattaattacaaaatagtagtctattttgatctatcacggactattgtgggtattttataatattttgttatattttataaatatttttagttcattttgctatatttgaaaataaccatatattatttatattttatttttctagaaaatattttccataaactcaagttctatatttaaaatatatattcaaaagtttcctattacaaccctaagttgaatacttatagaattgtaataaaataagataattaactaaataagtctaattaaacactaataatttgaattaacaacactaaacaaatattgtcaacaaaacttagttcaattgacatctatatgaagaatcgagttccaaatcttcacacctgaacattaacaaaattatatagtaattatcttaattaattctcctcatcgataaagtgaatatctaattaaaaatttaaagtcaaaagtgtgaatttcttgaaatatcaaattaagacaaaattcaaatcaatttgaaaacatataaacaaaatggtaaattagacaaaaaaaaaaaatcctaaaaactacatatgaaaaggttcattaccaaagaagtttttccatgaaaaaaaaaaagaaagaaagagaataaaatattatatatagttaataattatgaaatttttgtataatcccataaagtttgcaactaaacttaagcatatagtttatgacataattaaggtcactaataatagagaacagttagagcaaaggtcaaacatccactttattcactctctctcaatcatacaaagagatttaattgaatctactcattacaaaatccccaatcttataataatattaatatcattaatctcttatatatatatataatatatatacatatattatctcatgcacatggattttcatgatcttcaaaccccacgtcgttgattttccataaaacctatatattccactaatcatttatattcatttttttttttgggtctaattttaaactatatgttttaaaactccatagtttgatcaattcaaaaaaaaaaaaaaaaaaaagtgagttatacaatttttaaaatttttaggacataatcttgacaagtatctttatctctcctacatgaaagagggagcataagattagcttgacattgtctaaaattggaagtgtatatatatatatatatatatctataaatttagaaattaaaataatggggttttttcatgaaatatatattaatagcttgattaaggaaggtttagagggtgattaaagtgcaataatattgttgattaattgttttttttttcttatgtgtatcttagtttcaaggactcatgttttttttttcttttttctttttggtcccatggaagagaactttttttcaattataggatttgggtttttagtttttgggaattattgaaaagttataatttctgttgctaatgatgggaaaattatgaaaaattatatatgcatgggttggtggggtcataagatctcaaagaagcttttattttgtcattatttttctttagaaaatcagaatcttaatctttttttttttacacattggtattttggtcccctctcgtccaacccaaatttaaaaaagatcaaaaaagaaaaaaaaaaaaaaaagaaacagaaacctaatcttaaatcaatttccactatgcaatccttaattgtcatgttgatataaaaaaaaatagtaacgaggcaggagattgaaccataaaacttagctttgtggttattaatacacttagatgatgctaattgagttaaactcttgattgacaattaaaagaaaagttaaatcattagttaaataattaaagtttaatgatcataagttaatatttgatgttgggtattaataaaggagatgcatttgactaaaaaaatgattaggtagagactagggtaattaataaccaatattaataaagtatggttatgggggaattcatgacaaactcaagaggggatgttcatttgggtcttaatgaagtgtaggaattcaaataatttaaaaagttattaataattattatgattttattattattatttcatttgggtctacataagtataaagaattgattaaagaggttgattatgcagaaagaagggtgattagagaagtacaattatgaagggattttggataaacacataggaacgaatgattttcattgggggccttaacaaataatattcaattttaaaaaaattgactatttgcaattaggtcttgatcatgaagatcctcgagataaattatagttttttctttttttcttcgcatatgaatttgttcgatataacgaattttccgacatatcttacgtacactgataagatattgtctgcttaggatctatacttgtgatttattctattatctaatcaatgtgagattttggtctcattcctaacaattctctgctaattaattgaacaaaggacgatcactgaggctccattcaaataggaactcttatatctaggttaattactatgctacattagaacatatcacctatctgatagagttcaaacacatatcacaccatgagtactactttttgaggctaagctccactacatctttgtttgacacccaaatactctatctacacgactaggttaggagcataaactttgataccatctctttgagacataaactcccgtcactttatttttcatttcattgatctaaaacgtcttataccaatagagatagttgttttcacatatatatacttatattatcctattgcctagtgaatctttatacaaagcaacatactttaattttgattaaacaaagagtgattacacatggagatcatagcctaattaaataattaaagtataattatagggagggattttgagagaaatgtaattcaacaaggattttgcataagggtcttagataaggaactaaacaactagaaaaaaaaatataatatatatatatataaaagggaaatgaaatcaaagaaagcatccattctccatatatataaaaatacatatatatatatggggaagagagaagagattacaaaactaatttaataataaggtagttgagggggcaaaaagcaaaatacaagagattttgatttttgagagaagccctttttagcaaaaaaaataaaatagattaatataacacacaaacacacacctactccttttcttcaaccaccagattcgattttgcctctctctctctctctctctctctctctctctgtggatcttaaaccccaattcaaaatatgatgacaaattattaattattattcctccaaaaatattttccctattaaaaaaaataccaagagagagaaaattcaatgattgttttttctcttttacattatttttcttttaaagaaaaaaacttgctataaatagaggtgcccattgtaagagcaacattcaattcaacaaatcttcagttcaatttctctctttttggctctcaaaaagggaaagaaaaaaaaatcattattattattatttcattttctttctttcccttaaatttgagctgaaggaaaaaaaaaaaaaaaaaatcaatggcgattgagattgatattgagcaaaatccaacggttgaactttcgcgaatcggaacatcagaaacacacggcgaagattcgccgtattttgctggctggaaagcgtatgatgaagatccttataatgaatcaacaaatccttctggtgttattcaaatgggcttagctgaaaatcaagtaagaatatataacttttttttgttttgttttgctttgtaaggagattgggtttttttttttaattgggtttgtgttggaatttatgaaacaggtgtcatttgacttattggaggaatatttggaggaaaattgtgagggagaagggaattatttaaattctgggtttagagaaaatgctttatttcaagactatcatggtcttttctcatttagaagtgcaatgggaagttttatggaagagattagaggtggaagagcaaaatttgacccaaatcgagttgttttaactgctggtgccactgctgccaatgagcttctcactttcattcttgcaaatcctggcgatgctttgcttgtccccactccttactatcctgggtaagtttatcatcacctctacgttttcgtatttcatttcaaaaaccactctttactgtaattactataccctcagacattaaaattttaactttcaaactattcttaaagtatgagtttgagggtatttcatatggggtttttaaatgtaaatttatttacatttttccactacttaagtgtcctatatttctactaatttcttcttgtgttgtactcatattttctatcgtggggtggactacgtatttttacgagactattcgtataacatacgaatgagtgctttttaaaccaaattcttcaaaatccaagtttaattttggaaactagaaaatgggtagttttttaaaatgttaccaaacgtgatctttatccttacaatcaaacattaccaaggataattgcaactaccgttagactttatgagtgcttttttttccaactgttctatatttttacaacattttgagttgtattcatcatttctgttaaagatatttatatgtaactaagtatttttataagacactgttggtataatttcatgcactaataatatagtttctttttccagatttgacagagatttgagatggagaacaggagtgaaaattgtaccaattcattgtgacagttcaaacaattttcaaataactccaaaagcattagaagaagcttataattcagcaatggaaatgaaaatcaaagtaagaggagttttaatcacaaatccatcaaatccactcggagcaacgatccaacgctccacaatcgaagacattctagatttcgttacacgcaaaaacatccacctcgtatccgacgaaatctattccggttccgttttctcctccgccgagttcacaagcgtcgctgaggttttggaatcccgcagctacaaaaacgccgaacgtgtccacatcgtttacagcctctccaaagatctcggccttcccgggtttagaatcggcacgatctactcatacaacgataaagtcgtcacaaccgctcgccggatgtctagctttacgcttatctcttcacaaacgcaacgatttttagcgtccatgttgtcgaaccggaagtttacggagaaatatattaaaatgaaccgggacaggctcaagaaacggtatgaaatgattattgaagggctgcgaaccgccgggattgaatgtttggaagggaatgccggtttgttttgttggatgaatttgagcccgttgttgaaagataaaaaaaccaaagaaggtgagattgagatatggaagaggattttgaaggaagtgaaattgaatatttcgcccggttcgtcgtgtcattgctctgaacccggttggttcagggtttgttttgctaatatgagtgaaaagactctgcatgttgcccttgatagaatacgtcggttcatggaacggatgaagaaggaaaacgaagctaattaaatatatatctatatataaatatatgaaaagaaaaaaaacatatgtagcttattttattttatttttttttacaatggttgtgagaaaaaagaaaaaagaaaaaaaaaagaaaaaaaagccattgtgattcttttgtgtggacactgcccaatatttgttagaaatttggggttttttgtcttcatttatacgtcatattttgatgatttaaactgaggaaaaagaaaaagaaatccttgttttcttgcttttagcaaagcaagttttatttctcagttttatatatatatatatataaagtttctatttgtattgtcatttttatgtgatatggaatataattagtataattcgttcttgcaattaattacctcgaaaataaacgaaatacaagaaaaagaaaaaaaaaatctcatggagtattttagggacaagtgtcaactcagggagagagaaaaaaatatggtttaaatttaatagtattggttattttcataacatgctctaaaaaggaatataactaataatttgactttaattaagaaaagaaaagctaataatatataattaaaatcacttttagcaacgaataacactttgccgacttgtgtaattaaccacctaactatccatctgacgtggaatgcaagtaattaattaattgattttttcgttttcaaattttggtcaacttgattcttggtactaatttaatgtttccatctgtcagaaagctacaacgtttttcctccttctttcttttttttaagaattattttaaaaagtcaatacggtgctataattagatttttatttttcctcttttttagtgtatatatatatttatataagtagagattaggaactaattgattgaaaattaaatatgctgtgacgctcaaaagatattaatcccgcgttggttttatgtatttaaaaaatgtattttttcttttttgatatttttaaataataaaatatttaaattatttacaaaatataacaaaatttgctcgtttacatttttattttgtgaaggacttatgcgatgtggttcgatctagaattcttgtattttcaaaatagatggagcttctttttggatgaattctctctaggcttctgaagtcaaaaattttcaacccaagaaaaaactagagtttccttgtggtatgaggtgtatgaaattgactcattgactcaattacatggacttttatcatatttaactcagctaaattaagtttattttttggaattaatctaagtaaataatatttaattgaaccaaaatatttaatttgatcagtcataataaagacatgtgacatcattggaatcagtcaatttgtgtttaaatttaatttgggatacatgtcaacttttagttaatctcaaatgcaatttgtgattagttacaaaatttcttattcaacatacttcaaatctaaatttggtaaattatgttttttttaaagaaattagatcaacacaaaaatataatatgttttgtgaaaatgaaaattttggtttaatgggaggagacaaatttgaacgacaaattttcttagtaacttacgatatgatcactaactaatttattataggttgggggtttgaacctctctcaactttgtgctcattatataatatatctttaaaagaccgaccttgcattaatgttgttggttagtctagtggtagaatcgtcattctctagctctctctaaattgttccagcttcagtttttatatacttttttatattatttttactgaactataaaaaattactgtcgacaaaatttatgcttttatcacttaacataataattgaactatgtttgctttgttttttcttttttaacgtatactatctcaaagttttggataatgtacgtgtttgaaattttgtaacaaacaaggatacataaatacgtaattgttgttaattatttcaaaatgtaaatagatgatatgatgtaggggttgctaatattattgtctaattatttttgtaaagaataaaataaaataaaaatccatatgatgcctaaggacgtgtttagtattcaatatggcgattattatgtctttttttcaaatggattactttttggatgatatgatatcttttattttaattaaaactttttgatgacttttaaaatttaaagggcataaaatatagctttcttttgtacatatatatggttgtgcttggattttgtatctgcttgtctttgtcccgagtttctcgtcggtttgaccttcgatctactttttttgatatatatgttccaatatcgatttctaagctaacatatgtaaaagatgattgcactcgtgtggtgctgagactatcacacttgatacttgatataagattgtattattcatctgaaccaataaaaattaagatggatattactcactcttctcttttaggatcatcaaacaaggctctttttttgacaatcacctgaagtcacgagtaactctacagtgttatagttgtttatagtatttgagactggaaatatcaaattcagccaattcaattaaattttataattgatatacattcatatgctcgaaaagttggcttccaaccgtccaacgctctaactttgggacatgtgttacaaaacacataggtttaaaaaaatacctaaaaacataaaaaatacaactttgatcatcctttagttcaattttattcccaatttgatacttttgaattccctaactagagaattgtaattgtgattgaacgtttttatagtcaaaatttggatttgatagcttataggaccccatgtgccaaacaaaataaaaattgcatatatatatatagagagagagggggagttttatattttaaaaaacaaaattagaaaaaggcaattaattttgttctttaaacgacatggtttctcacgtgttaaacgtcattgttatcaactgcgtcttgtcgctgaccaattcattgacagcacagcaagcaaaaaagaaaaaagaaaaaaaaaagtcaaaaagttaatgtggttaaagaaagcgctttttgtgataactcaaaaaaacaaaccaaccaatggaagccctccaggtcactctcatgtggctcccgtctccacgtcatcaatgaacacggacgtgtttacagctcaacaaacaacaactattcctctgacgtggcaatttcttattaatgaatttccaactctgccctcctattacattttcttaaggtcggtgaatgccgtacaatggaagctcctagacatactttctagttgtgatatatatatatatatattattcatccatcatatattaaactccaatgtatgtttgtggattttacaaaagttaatattatttggtagatgttgagatttcttttttttccaatgtctagtctttttccaagtttggagaaagttttttatgatgttgggagttatttaatttcctagtggggccacgtagttaaataaatataggttaaattttacgaaatatccatatatcgcgtgtgtggtatgatttcagttacgtcactattttgaaaacatagttttcgtgttcttattaatgcttatagttgtaaataacataattaaaagtatcatttgttaaaattgatgtcacaccgtatgtacataatttatttattgattgatgttataaggggcgttggagatatcgttggaaaaaaatgatttgtaaggatgatcttaattttctataattgactcacgtatattatattgtatacgtttttcaaaatttacacaccaatcatctcactttcgttttcatttttcattttagtggaaaacaattcaataaaaaaaaattctgacaactttttaaaatttaaggcacagttgaatcaatccaaccgttcaagatttaaagaagaaaaaaactaatttggttgctccactttttgtttttgttcgttttggtccattaattctaaaaatgtttaatttatttgttatactttcaaatcttcacaactttaccgtattgatcccttaaaaatgaagtaaaaacaataatgaacgaactaagacaatcaccatttgaaagtttaaggaccaaatgaaccaaagttaaaagtatagaaacaaaaataaacatcgctaaataaaccaaataaaactagaattacttaattgaaacaaaataatatgaaatggatcaaatctttagactttagtgtatgggaaagttctatgaaaatgaccaccgactatcgagagaccaattttggggccaagtcaatgattggtaatttcaacctacatttatgatgtatgacaatgacaatagcttaggtcactttgaaaatgactataagattttctagttagagatatacacttgatattagacttggtcgttgtaataaaaactatgtgtcacggatgatatatgctaagtacatgttttagtctttaatgtttgcgtatatttctttacgtaatttaatcttcgttaattatattttttaaactatgttttaatcttttaattcttttgttgtgaaattgacaaataaagagaaacacgacaatgtagatggtaaacaatgaagtttgtgtagtttattgacaatatgttggcaatgtttacgagtagagagataattgttcaaattacagaacaataggtgacaatacgtgagtttttgttttaatttacttttgaaactttattttgattttcaaaacttaaagaagttgatactattattgttttgagctatgaagatgtgtgatcgaacctttcacacgtttagaatgaaagagcatgtcaattaattttgagctaaacttgtttaaaaaaattgaccttttgtctttgttttaagttttaacaaattaatgatgtcattgcgtaattttaagtcagttaggtatgaaaagccaccatcgaagaaagaaaatttcaagaagaaaagcaatgtagtaaatcacaaataattgtttttctttcccataggttataactataaaaaaaaaacttcttttttagtataataacggtaaagaaggatgatcaaaccttctaactcagtcaattgcaaatatgataaattcactttgacaaactaaaataaatttgaagatttatgaaacaaaatgtacattttaaaagtttatattttcacacaatgttagcttccttttaaaaaaaaattaaattttaaaagttcagagaacaaaacatacatttcaactttgctaacttcaatatagaacttatataaaatcgtgccacatagggttcaaaagaactataggattttaaaatgaaaacatatattttaattgagttttagaactaagttaatatatttatttataaagaataacacttcagtaaaattaagaacaacacagacatagttcaataacataaaactagtaccttttacatcaatattagaa

SEQ ID NO: 2 - DNA, 11137 bp, Cucumis melo
tagtctattttgatctatcacgaactattgtaggtattttataatattttgttatattttataaatatttttagttcattttgctatatttaaaaataaccatatattatttatattttatttttctaaaaaatattttccataaactcaagttctatatttaaaatatatattcaaaactttcttattacaaccctaagttgaatacttatagaattgtaataaaataagataattaactaaataagtctaattaaacattaataatttgaattaacaacactaaacaaatattgtcaacaaaacttagttcaattgacatctatatgaagaatcgacttccaaatcttcgcacctgaacattaacaaaattatatagtaattatcttaattaattatcctcatcgataaagtgaatatctaattaaaaatttaaagtcaaaagtgtgaatttcttgaaatatcaaattaagacaaaattcaaatctctttagagtaaaattatagaatttgaaaacatataaacgaaaaatggtaaattagacaaaaaaaaaatatattttttctaaaaactacatatgaaaaggttcattaccaaagaagtttttccatgaaaaaaaaaagaaagaaagaaagaaagagaataaaatattatatatagttaataattatgaaatttttgtataatcccataaagtttgcaactaaacttaagcatatagtttatgacataattaaggtcactaataatagagaacagttagagcaaaggtcaaacatccactttattcactctctctcaatcatacaaagagatttaattgaatctactcattacaaaatccccaatcttataataatattaatatcattaatctcttatatatatatataatatatatacatatattatctcatgcacatggattttcatgatcttcaaaccccacgtcgttgattttccataaaacctatatattccactaatcatttatattcatttttttttttgggtctaattttaaactatatgttttaaaactccatagtttgatcaattcaaaaaaaaaaaaaaaaaaaaaagtgagttatacaatttttaaaatttttaggacataatcttgacaagtatctttatctctcctacatgaaagagggagcataagattagcttgacattgtctaaaattggaagtgtatatatatatatatatatatctataaatttagaaattaaaataatggggttttttcatgaaatatatattaatagcttaattaaggaaggtttagagggtgattaaagtgcaataatattgttgattaattgtttttttttcttatgtgtatcttagtttcaaggactcatgttttttttttcttttttctttttggtcccatggaagagaactttttttcaattataggatttgggtttttagtttttgggaattattgaaaagttataatttctgttgctaatgatgggaaaattatgaaaaattatatatgcatgggttggtggggtcataagatctcaaagaagcttttattttgtcattatttttctttagaaaatcagaatcttaatcttttttttttacacattggtattttggtcccctctcgtccaacccaaatttaaaaaagatcaaaaaagaaaaaagaaaaaaaaaaaaagaagaagaagaaacctaatcttaaattaatttccactatgcaatccttaattgtcatgttgatataaaaaaaaatagtaacgaggtaggagattcaaccataaaacttagctttgtggttattaatacacttagatgatgctaattgagttaaactcttgattgacaattaaaagaaaagttaaatcattagttaaataattaaagtttaatgatcataagttaatatttgatgttgggtattaataaaggagatgcatttgactaaaaaaatgattaggtagagactagggtaattaataacaaatattaataaagtatggttatgggggaattcatgacaaactcaagaggggatgttcatttgggtcttaatgaagtgtaggaattcaaataatttaaaaagttattaataattattatgattttattattattatttcatttgggtctacataagtataaagaattgattaaagaggttgattatgcagaaagaagggtgattagagaagtacaattatgaagggattttggataaacacataggaacgaatgattttcattgggggccttaacaaataatattcaatttttaaaaaattgactatttgcaattaggtcttgatcatgaagatcctcgagataaattatagttttttcttgttttcttcgcatatgaatttgttcgatataacgaattttccgacatatcttacgtacactgataagatattgtctgcttaagatctatacttgtgatttattctattatctaatcaatgtgagattttggtctcattcctaacaattctctgctaattaattgaacaaaggacgatcactgaggctccattcaaataggaactcttatatctaggttaattactatgctacattagaacatatcacctatctgatagagttcaaacacatatcacaccatgagtactacttttcgaggctaagctccactacatctttgtttgacacccaaatactctatctacacgactaggttaggagcataaactttgataccatctctttgagacataaactcccgtcactttatttttcatttcactgatctaaaacgtcttataccaatagagatagttgttttcacatatatatacttatattatcctattacctagtgaatctttctgcaaagcacatactttaattttgattaaacaaaaagtgattacacatggatatcatagcctaattaaataattaaagtataattatggggggggggggattttgagagaaatgtaattcaacaaggattttgcataagggtcttagataaggaactaaacaactagaaaaaaaatataatatatatatatataaaaaagggaaatgaaatcaaagaaagcatccattctccatatatataaaaatacatatatatatatggggaagagagaagagattacaaaactaatttaataataaggtagttgagggggcaaaaagcaaaatacaagagattttgatttttgagagaagccctttttagcaaaaaaaataaaatagattaatataacacacaaacacacacctactccttttcttcaaccaccagattcgattttgcctctctctctctctctctctctctctctctctctctggatcttaaaccccaattcaaaatatgatgacaaattattaattattattcctccaaaaatattttccctattaaaaaaaataccaagagagagaaaattcaatgattgttttttctcttttacattattttttttttaaagaaaaaaacttgctataaatagaggtgcccattgtaagagcaacattcaattcaacaaatcttcagttcaatttctctctttttggctctcaaaaagggaaagaaaaaaaaatcattattattattatttcattttctttctttcccttaaatttgagctgaaggaaaaaaaaaaaaaatcaatggcgattgagattgatattgagcaaaatccaacggttgaactttcgcgaatcggaacatcagaaacacacggcgaagattcgccgtattttgctggctggaaagcgtatgatgaagatccttataatgaatcaacaaatccttctggtgttattcaaatgggcttagttgaaaatcaagtaagaatatataacttttttttgttttgttttgctttgtaaggagattggggtttttttttaattgggtttgtgttggaatttatgaaacaggtgtcatttgacttattggaggaatatttggaggaaaattgtgagggagaagggaattatttaaattctgggtttagagaaaatgctttatttcaagactatcatggtcttttctcatttagaagtgcaatgggaagttttatggaagagattagaggtggaagagcaaaatttgacccaaatcgagttgttttaactgctggtgccactgctgccaatgagcttctcactttcattcttgcaaatcctggcgatgctttgcttgtccccactccttactatcctgggtaagtttatcatcacctctacgttttcgtatttcatttcaaaaaccactctttactgtaattactataccctcagacattaaaattttaactttcaaactattcttaaagtatgagtttgagggtatttcatatggggtttttaaatgtaaatttatttacatttttccactacttaagtgtcctatatttctactcatttcttcttgtgttgtactcatattttctatcgtggggtggactacgtatttttacgagactattcgtataacatacgaatgagtgctttttaaaccaaattcttcaaaatccaagtttaattttggaaactagaaaatgggtagttttttaaaatgttaccaaacgtgatctttatccttacaatcaaacattatcaaggataattgcaactatcattagactttatgagtgctttttttttccaactgttctatatttttacaacattttgagttatattcatcacttctgttaaagatatttatatgtaactaagtatttttataagacactgttggtataatttcatgcactaataatatagtttctttttccagatttgacagagatttgagatggagaacaggagtgaaaattgtaccaattcattgtgacagttcaaacaattttcaaataactccaaaagcattagaagaagcttataattcagcaatggaaatgaaaatcaaagtaagaggagttttaatcacaaatccatcaaatccactcggagcaacgatccaacgctccacaatcgaagacattctagatttcgttacacgcaaaaacatccacctcgtatccgacgaaatctattccggttccgttttctcctccgccgagttcacaagcgtcgctgaggttttggaatcccgcagctacaaaaacgccgaacgtgtccacatcgtttacagcctctccaaagatctcggccttcccgggtttagaatcggcacgatctactcatacaacgataaagtcgtcacaaccgctcgccggatgtctagctttacgcttatctcttcacaaacgcaacgatttttagcgtccatgttgtcgaaccggaagtttacggagaaatatattaaaatgaaccgggacaggctcaagaaacggtatgaaatgattattgaagggctgcgaaccgctgggattgaatgtttggaagggaatgccggtttgttttgttggatgaatttgagcccgttgttgaaagataaaaaaaccaaagaaggtgagattgagatatggaagaggattttgaaggaagtgaaattgaatatttcgcccggttcgtcgtgtcattgctctgaacccggttggttcagggtttgttttgctaatatgagtgaaaagactctgcatgttgcccttgatagaatacgtcggttcatggaacggatgaagaaggaaaacgaagctaattaaatatatatatatatatatataaatatatgaaaagaaaaaaaacatatgtagcttattttattttattttttttttacaatggttgtgagaaaaaagaaaaaagaaaaaagaaaaaaaaaaagaaaaaaaagccattgtgattcttttgtgtggacactgcccaatatttgttagaaatttggggttttttgtcttcatttatacgtcatattttgatgatttaaactgaggaaaaagaaaaagaaatccttgttttcttgcttttagcaaagcaagttttatttctcagttttatatatatatataaagtttctatttgtattgtcatttttatgtgatatggaatataattagtataattcgttcttgcaattaattacctcgaaaataaacgaaatacaagaaaaagaaaaaaaaaatctcatggagtattttagggacaagtgtcaactcagggagagagaaaaaaatatggtttaaatttaatagtattggttattttcataacatgctctaaaaaggaatataactaataatttgactttaattaagaaaagaaaagctaataatatataattaaaatcacttttagcaacgaataacactttgccgacttgtgtaattaaccacctaactatccatctgacgtggaatgcaagtaattaattaattgattttttcgttttcaaattttggtcaacttgattcttggtactaatttaatgtttccatctgtcagaaagctacaacgtttttcctccttctttcttttttttaagaattattttaaaaagtcaatacggtgctataattagatttttatttttcctcttttttagtgtatatatatatttatataagtagagattaggaactaattgattgaaaattaaatatgctgtgacgctcaaaagatattaatcccgcgttggttttatgtatttaaaaaatgtattttttcttttttgatatttttaaataataaaatatttaaattatttacaaaatataacaaaatttgctcgtttacatttttattttgtgaaggacttatgcgatgtggttcgatctagaattcttgtattttcaaaatagatggagcttctttttggatgaattctctctaggcttctgaagtcaaaaattttcaacccaagaaaaaactagagtttccttgtggtatgaggtgtatgaaattgactcattgactcaattacatggacttttatcatatttaactcagctaaattaagtttattttttggaattaatctaagtaaataatatttaattgaaccaaaatatttaatttgatcagtcataataaagacatgtgacatcattggaatcagtcaatttgtgtttaaatttaatttgggatacatgtcaacttttagttaatctcaaatgcaatttgtgattagttacaaaatttcttattcaacatacttcaaatctaaatttggtaaattatgttttttttaaagaaattagatcaacacaaaaatataatatgttttgtgaaaatgaaaattttggtttaatgggaggagacaaatttgaacgacaaattttcttagtaacttacgatatgatcactaactaatttattctaggttgggggtttgaacctctctcaactttgtgctcattatataatatatctttaaaagaccgaccttgcattaatgttgttggttagtctagtggtagaatcgtcattctctagctctctctaaattgttccagcttcagtttttatatacttttttatattatttttactgaactataaaaaattactgtcgacaaaatttatgcttttatcacttaacataataattgaactatgtttgctttgttttttcttttttaacgtatactatctcaaagttttggataatgtacgtgtttgaaattttgtaacaaacaaggatacataaatacgtaattgttgttaattatttcaaaatgtaaatagatgatatgatgtaggggttgctaatattattgtctaattatttttgtaaagaataaaataaaataaaaatccatatgatgcctaaggacgtgtttagtattcaatatggcgattattatgtctttttttcaaatggattactttttggatgatatgatatcttttattttaattaaaactttttgatggcttttaaaatttaaagggcataaaatatagctttcttttgtacatatatatggttgtgcttggattttgtatctgcctgtctttgtcccgagtttctcgtcggtttgacctccgatctacttttcttgatatatatgttccaatatcgatttctaagctaacatatgtaagagatgattgcactcgtgtggtgtcgagactatcacacttgatacttgatataagattgtattattcatctgaaccaataaaaattaagatggatattactcactcttctcttttaggatcatcaaacaatgctctttttttgacaatcacctgaagtcatgagtaactctacagtgttatagttgtttatagtatttgagactagaaataacaaattcagccaattcaattaaattttataattgatatacattcatatgctcgaaaagttggcttccaaccgtccaacgccctaactttgggacatgtgttacaaaacacataggtttaaaaaaatacctaaaaacataaaaaaaaatacaattttgatcatcctttagttcaattttattcccaatttgatacttttgaattccctaactagagaattgtaattgtgattgaacgtttttatagtcaaaatttggatttgatagcttatagaaccccatgtgccaaacaaaataaaaattgcatacatatatatatatatatatatatatatatatatatatatatatatatatatatatatagagggagttttatattttcaaaaacaaaattagaaaaaggcaattaattttgttctttaaacgacatggtttctcacgtgttaaacgtcattgttatcaactgcgtcttgtcgctgaccaattcattgacagcacagcaagcaaaaaagaaaaaaaaaaaaaaaaaagtcaaaaagttaatgtggttaaagaaagcgctttttgtgataactcaaaaaaacaaaccaaccaatggaagccctccaggtcactctcatgtggctcccgtctccacgtcatcaatgaacacggacgtgtttacagctcaacaaacaacaaccattcctctgacgtggcaatttcttattaatgaatttccaactctgccctcctattacattttcttaaggtcggtgaatgccgtacaatggaagctcctagacatactttctagttgtgatatatatatatatatatatatattattcatccatcatatattaaactccaatgtatgtttgtggattttacaaaagttaatattatttggtagatgttgagatttcttttttttccaatgtctagtctttttccaagtttggagaaagttttttatgatgttgggagttatttaatttcctagtggggccacgtagttaaataaatataggttaaattttacgaaatatccatatatcgcgtgtgtggtatgatttcagttacgtcactattttgaaaacatagttttcgtgttcttattaatgcttatagttgtaaataacataattaaaagtatcatttgttaaaattgatgtcacaccgtatgtacataatttatttattgattgatgttataaggggcgttggagatatcgttggaaaaaaatgatttgtaaggatgatcttaattttctataattgactcacgtatattatattgtatacgtttttcaaaatttacacaccaatcatctcactttcgttttcatttttcattttagtggaaaacaattcaaaaaaaaaaaaaattctgacaactttttaaaatttaaggcacagttgaatcaatccaaccgttcaagatttaaagaagaaaaaaactaatttggttgctccactttttgtttttgttcgttttggtccattaattctaaaaatgtttaatttatttgttatactttcaaatcttcacaactttaccgtattgatcccttaaaaatgaagtaagtcaatcaccatttgaaagtttaaggaccaaatgaaccaaagttaaaagtataaaaacaaaaataaacatcgctaaataaaccaaataaaactagaattacttaattgaaacaaaataatatgaaatggatcaaatctttagactttagtgtatgggaaagttctatgaaaatgaccaccgactatcgagagaccaattttggggccaagtcaatgattggtaatttcaacctacatttatgatgtatgacaatgacaatagcttaggtcactttgaaaatgactataagattttctagttagagatatacacttgatattagacttggtcgttgtaataaaaactatgtgtcacggatgatatatgctaagtacatgttttagtctttaatgtttgcgtatatttctttacgtaatttaatcttcgttaattatattttttaaactatgttttaatcttttaattcttttgttgtgaaattgatacaaataaagaggtttctctttattgacaatatgttgcgtagtttattgacaatatgttggcaatgtttacgagtagagagataattgttcaaattacagaacaataggtgacaatacgtgagtttttgttttaatttagttttgaaactttattttgattttcaaaacttaaagaagttgatactattattgttttgagctatgaagatgtgtgatcgaacctttcacacgtttagaatgaaagagcatgtcaattaattttgagctaaacttgtttaaaaaaattgaccttttgtctttgttttaagttttaacaaattaatgatgtcattgcgtaattttaagtcagttaggtatgaaaagccaccatcgaagaaagaaaatttcaagaagaaaagcaatgtagtaaatcacaaataattgtttttctttcccataggttataactataaaaaaaaacttcttttttagtataataacggtaaagaaggatgatcaaaccttctaactcagtcaattgcaaatatgataaattcactttgacaaactaaaataaatttgaagatttatgaaacaaaatgtacattttaaaagtttatattttcacacaatgttagcttccttttaaaaaaaatttaaattttaaaagttcagagaacaaaacatacatttcaactttgctaacttcaatatagaacttatataaaatcgtgccacatagggttcaaaagaactataggattttaaaatgaaaacatatattttaattgagttttagaactaagttaatatatttatttataaagaataacacttcagtaaaattaagaacaacacagacatagttcaataacataaaactagaggatc

SEQ ID NO: 3 - protein, 445 aa, Cucumis melo
MAIEIDIEQNPTVELSRIGTSETHGEDSPYFAGWKAYDEDPYNESTNPSGVIQMGLAENQVSFDLLEEYLEENCEGEGNYLNSGFRENALFQDYHGLFSFRSAMGSFMEEIRGGRAKFDPNRVVLTAGATAANELLTFILANPGDALLVPTPYYPGFDRDLRWRTGVKIVPIHCDSSNNFQITPKALEEAYNSAMEMKIKVRGVLITNPSNPLGATIQRSTIEDILDFVTRKNIHLVSDEIYSGSVFSSAEFTSVAEVLESRSYKNAERVHIVYSLSKDLGLPGFRIGTIYSYNDKVVTTARRMSSFTLISSQTQRFLASMLSNRKFTEKYIKMNRDRLKKRYEMIIEGLRTAGIECLEGNAGLFCWMNLSPLLKDKKTKEGEIEIWKRILKEVKLNISPGSSCHCSEPGWFRVCFANMSEKTLHVALDRIRRFMERMKKENEAN

SEQ ID NO: 4 - DNA, 19 bp, Artifical Sequence
  Synthetic Construct primer
gatgagtcctgagtaagta

SEQ ID NO: 5 - DNA, 19 bp, Artifical  Sequence
  Synthetic Construct primer
gatgagtcctgagtaatgt

SEQ ID NO: 6 - DNA, 18 bp, Artifical sequence
  Synthetic Construct probe
gactgcgtacatgcagca

SEQ ID NO: 7 - DNA, 27 bp, Artifical sequence
  Synthetic Construct probe
cgtattttgctggctggaaagcgtatg

SEQ ID NO: 8 - DNA, 27 bp, Artifical sequence
  Synthetic Construct probe
cgatagaaaatatgagtacaacacaag

SEQ ID NO: 9 - DNA, 16177 bp, Artifical sequence
  Synthetic Construct vector pEC2 with insertion of the ACS gene
ggaaacagctatgaccatgattacgccaagctcggaattaaccctcactaaagggaacaaaagctggagctccaccgcggtggccggggccgctctagccacagacagctccgtagccctcgttctccttggagttcttcgggaaatggatctttcgattcccgatgatgtctctcttatctgctttgacgacgccgactggacatccgctataacgccgccattgaccgtgatttcgcaacctgtcagggatctcgcgacggctgccacagaagacctgatcgcccgcttaaagggcgagacttcagccccacccaaggaaactcttctcccggcggttctcatagagcgcggttccgtaagcggttcttcgcaaggtcggggttgcataccgaactcgcgaaacgtcggcgactgagctcccgaggcgcgttgacaagatgccacgaagggaatggaagacagccgatattgcaattgtcttcgtggactgctttcgggacgtaaggcgcaagccatcatcaccgccgtcctaaacaaacatacctccacacaaatttatctacctgaccacaagatatatcctgtcacacgatttattaaacgctgcacttggctagaactagtggatccgcggccgcatgcctgcaggtcgactctagttgattaattgtttaggctttattatatattcatcataagtcttttttgtagccatttaggtttgttttcgtcgaattaatcttataaacactatttttattcgtaaattccgttgctttcttatttactttatatcaatgctttaaaacatcaatctagtttttaaaaatcaatatatatgtttgcacacaccattattatcgtatgttactctatctattactgacaaacgttatgaaattttattatatttgtaattatcttttgcagttttgtcatttaaaatcgtttttcttaaaagaattatgttgttattttaaaattttggctaaagaatcacgtggagaattagatatatcaaacctttcatctttgagatgaaagattacatcaattactattaactaagcttactttgataaattaaaatcatattaaaacaaatagtccgtaaaagaatataattttgaaaaactaaacagtcatcaaacaacgcgtgttagcttttaatatatattatgatatgttaagtgaaaataaagttgaagtgtatgaagccaaaagagaagtcgttttcacttgttgagttctaatttctaggatggttctatgtaaagtacttcctcttccaaaattggaatccaactcactacttataaacatcatttattcgtcatcttaattacaataccaactcttatttttgtctcatctatcatcacactcactaattaacattaccattatcttatatcattttatgaactcattatttaacaaataaatcacttaaaagtttaacttcaaaaaaaaaaaggaagaaagaaagaaggtttgaaattacactatttgcaattaattatgttttatgaaaactttctaatactttaattttatgtcgaatcgtttgtcgaatcgtttctcttttatcctactacaaaaatattataaaatgattataaatggctaaaatatatagtatgtgtatttcataaatttaagaaaatgttttcgaatacagtcaaatgaactaaaatatttacaaaaatataacaaaatttcatatgtatatcgaataaaatttaaaaatttgaagactaaatttgtaatataattacatattaaagtaatttttagatgtgtgggtattatataataataatgttgggaaggtgagggcatgaggcagctggagggataaggactagggattgttttatatcctttttcacatttaatttttgatgctaattaatttgttgccaatcatttcatcacttttttttttttttttggttctaatttatttactttatatggaaaataaataaaagaaaaatgaaagaaagaaaaaagtggttttcaaatcaatagaaaaaacaaacaactccaactttaatggcttgaaaacaaatgcattctaaaattaaaccttatgattgatttgatttttattccccttttttacacttttcattttcatcataattatatcttcagttacctgtccaccaattacaccatcaaatgtggattattgggattcttttttttttttttaagattatcttacggctttcatttttttcgtatctttatgacggtttgatagacgtaaaagtggttattgtgttatagagatttgtattattttgatattatggaaggattcgtttgagtaaaattataaaaatcagaggggtgtcgtttaaaaatgtaagtaatccaacacaaaaaaataattatcataaaatgtaaaaaaaagggttagattgaaaacaaacgaaacaaatgagttttgtattataaatcgacctaaaatgttcaacccaaacatggattacgatacgaccgattcatctcattacagctcatcgatcctaaaaatgtgaagagaagtattggatataattattacttaaaaagataatagaaaaaggaaatcagcaaaattagggttctttaataagttataaaactcatttatatacaaaattaattacattacaaaaggtgggaatgtggatttagacatacaacctataataattaattaaaaacaatacacatgtttcacaatttgagataattaaattttaatccccatttgataagtaatgattttatcttataaattagtttgttaggtctatactttatttgtttatttatttattcttactcttttttaattatatttttacttatatcccaagcttcattaacgattaatctaagtttgaaatgattaattacaaaatagtagtctattttgatctatcacggactattgtgggtattttataatattttgttatattttataaatatttttagttcattttgctatatttgaaaataaccatatattatttatattttatttttctagaaaatattttccataaactcaagttctatatttaaaatatatattcaaaagtttcctattacaaccctaagttgaatacttatagaattgtaataaaataagataattaactaaataagtctaattaaacactaataatttgaattaacaacactaaacaaatattgtcaacaaaacttagttcaattgacatctatatgaagaatcgagttccaaatcttcacacctgaacattaacaaaattatatagtaattatcttaattaattctcctcatcgataaagtgaatatctaattaaaaatttaaagtcaaaagtgtgaatttcttgaaatatcaaattaagacaaaattcaaatcaatttgaaaacatataaacaaaatggtaaattagacaaaaaaaaaaaatcctaaaaactacatatgaaaaggttcattaccaaagaagtttttccatgaaaaaaaaaaagaaagaaagagaataaaatattatatatagttaataattatgaaatttttgtataatcccataaagtttgcaactaaacttaagcatatagtttatgacataattaaggtcactaataatagagaacagttagagcaaaggtcaaacatccactttattcactctctctcaatcatacaaagagatttaattgaatctactcattacaaaatccccaatcttataataatattaatatcattaatctcttatatatatatataatatatatacatatattatctcatgcacatggattttcatgatcttcaaaccccacgtcgttgattttccataaaacctatatattccactaatcatttatattcatttttttttttgggtctaattttaaactatatgttttaaaactccatagtttgatcaattcaaaaaaaaaaaaaaaaaaaagtgagttatacaatttttaaaatttttaggacataatcttgacaagtatctttatctctcctacatgaaagagggagcataagattagcttgacattgtctaaaattggaagtgtatatatatatatatatatatctataaatttagaaattaaaataatggggttttttcatgaaatatatattaatagcttgattaaggaaggtttagagggtgattaaagtgcaataatattgttgattaattgttttttttttcttatgtgtatcttagtttcaaggactcatgttttttttttcttttttctttttggtcccatggaagagaactttttttcaattataggatttgggtttttagtttttgggaattattgaaaagttataatttctgttgctaatgatgggaaaattatgaaaaattatatatgcatgggttggtggggtcataagatctcaaagaagcttttattttgtcattatttttctttagaaaatcagaatcttaatctttttttttttacacattggtattttggtcccctctcgtccaacccaaatttaaaaaagatcaaaaaagaaaaaaaaaaaaaaaagaaacagaaacctaatcttaaatcaatttccactatgcaatccttaattgtcatgttgatataaaaaaaaatagtaacgaggcaggagattgaaccataaaacttagctttgtggttattaatacacttagatgatgctaattgagttaaactcttgattgacaattaaaagaaaagttaaatcattagttaaataattaaagtttaatgatcataagttaatatttgatgttgggtattaataaaggagatgcatttgactaaaaaaatgattaggtagagactagggtaattaataaccaatattaataaagtatggttatgggggaattcatgacaaactcaagaggggatgttcatttgggtcttaatgaagtgtaggaattcaaataatttaaaaagttattaataattattatgattttattattattatttcatttgggtctacataagtataaagaattgattaaagaggttgattatgcagaaagaagggtgattagagaagtacaattatgaagggattttggataaacacataggaacgaatgattttcattgggggccttaacaaataatattcaattttaaaaaaattgactatttgcaattaggtcttgatcatgaagatcctcgagataaattatagttttttctttttttcttcgcatatgaatttgttcgatataacgaattttccgacatatcttacgtacactgataagatattgtctgcttaggatctatacttgtgatttattctattatctaatcaatgtgagattttggtctcattcctaacaattctctgctaattaattgaacaaaggacgatcactgaggctccattcaaataggaactcttatatctaggttaattactatgctacattagaacatatcacctatctgatagagttcaaacacatatcacaccatgagtactactttttgaggctaagctccactacatctttgtttgacacccaaatactctatctacacgactaggttaggagcataaactttgataccatctctttgagacataaactcccgtcactttatttttcatttcattgatctaaaacgtcttataccaatagagatagttgttttcacatatatatacttatattatcctattgcctagtgaatctttatacaaagcaacatactttaattttgattaaacaaagagtgattacacatggagatcatagcctaattaaataattaaagtataattatagggagggattttgagagaaatgtaattcaacaaggattttgcataagggtcttagataaggaactaaacaactagaaaaaaaaatataatatatatatatataaaagggaaatgaaatcaaagaaagcatccattctccatatatataaaaatacatatatatatatggggaagagagaagagattacaaaactaatttaataataaggtagttgagggggcaaaaagcaaaatacaagagattttgatttttgagagaagccctttttagcaaaaaaaataaaatagattaatataacacacaaacacacacctactccttttcttcaaccaccagattcgattttgcctctctctctctctctctctctctctctctctgtggatcttaaaccccaattcaaaatatgatgacaaattattaattattattcctccaaaaatattttccctattaaaaaaaataccaagagagagaaaattcaatgattgttttttctcttttacattatttttcttttaaagaaaaaaacttgctataaatagaggtgcccattgtaagagcaacattcaattcaacaaatcttcagttcaatttctctctttttggctctcaaaaagggaaagaaaaaaaaatcattattattattatttcattttctttctttcccttaaatttgagctgaaggaaaaaaaaaaaaaaaaaatcaatggcgattgagattgatattgagcaaaatccaacggttgaactttcgcgaatcggaacatcagaaacacacggcgaagattcgccgtattttgctggctggaaagcgtatgatgaagatccttataatgaatcaacaaatccttctggtgttattcaaatgggcttagctgaaaatcaagtaagaatatataacttttttttgttttgttttgctttgtaaggagattgggtttttttttttaattgggtttgtgttggaatttatgaaacaggtgtcatttgacttattggaggaatatttggaggaaaattgtgagggagaagggaattatttaaattctgggtttagagaaaatgctttatttcaagactatcatggtcttttctcatttagaagtgcaatgggaagttttatggaagagattagaggtggaagagcaaaatttgacccaaatcgagttgttttaactgctggtgccactgctgccaatgagcttctcactttcattcttgcaaatcctggcgatgctttgcttgtccccactccttactatcctgggtaagtttatcatcacctctacgttttcgtatttcatttcaaaaaccactctttactgtaattactataccctcagacattaaaattttaactttcaaactattcttaaagtatgagtttgagggtatttcatatggggtttttaaatgtaaatttatttacatttttccactacttaagtgtcctatatttctactaatttcttcttgtgttgtactcatattttctatcgtggggtggactacgtatttttacgagactattcgtataacatacgaatgagtgctttttaaaccaaattcttcaaaatccaagtttaattttggaaactagaaaatgggtagttttttaaaatgttaccaaacgtgatctttatccttacaatcaaacattaccaaggataattgcaactaccgttagactttatgagtgcttttttttccaactgttctatatttttacaacattttgagttgtattcatcatttctgttaaagatatttatatgtaactaagtatttttataagacactgttggtataatttcatgcactaataatatagtttctttttccagatttgacagagatttgagatggagaacaggagtgaaaattgtaccaattcattgtgacagttcaaacaattttcaaataactccaaaagcattagaagaagcttataattcagcaatggaaatgaaaatcaaagtaagaggagttttaatcacaaatccatcaaatccactcggagcaacgatccaacgctccacaatcgaagacattctagatttcgttacacgcaaaaacatccacctcgtatccgacgaaatctattccggttccgttttctcctccgccgagttcacaagcgtcgctgaggttttggaatcccgcagctacaaaaacgccgaacgtgtccacatcgtttacagcctctccaaagatctcggccttcccgggtttagaatcggcacgatctactcatacaacgataaagtcgtcacaaccgctcgccggatgtctagctttacgcttatctcttcacaaacgcaacgatttttagcgtccatgttgtcgaaccggaagtttacggagaaatatattaaaatgaaccgggacaggctcaagaaacggtatgaaatgattattgaagggctgcgaaccgccgggattgaatgtttggaagggaatgccggtttgttttgttggatgaatttgagcccgttgttgaaagataaaaaaaccaaagaaggtgagattgagatatggaagaggattttgaaggaagtgaaattgaatatttcgcccggttcgtcgtgtcattgctctgaacccggttggttcagggtttgttttgctaatatgagtgaaaagactctgcatgttgcccttgatagaatacgtcggttcatggaacggatgaagaaggaaaacgaagctaattaaatatatatctatatataaatatatgaaaagaaaaaaaacatatgtagcttattttattttatttttttttacaatggttgtgagaaaaaagaaaaaagaaaaaaaaaagaaaaaaaagccattgtgattcttttgtgtggacactgcccaatatttgttagaaatttggggttttttgtcttcatttatacgtcatattttgatgatttaaactgaggaaaaagaaaaagaaatccttgttttcttgcttttagcaaagcaagttttatttctcagttttatatatatatatatataaagtttctatttgtattgtcatttttatgtgatatggaatataattagtataattcgttcttgcaattaattacctcgaaaataaacgaaatacaagaaaaagaaaaaaaaaatctcatggagtattttagggacaagtgtcaactcagggagagagaaaaaaatatggtttaaatttaatagtattggttattttcataacatgctctaaaaaggaatataactaataatttgactttaattaagaaaagaaaagctaataatatataattaaaatcacttttagcaacgaataacactttgccgacttgtgtaattaaccacctaactatccatctgacgtggaatgcaagtaattaattaattgattttttcgttttcaaattttggtcaacttgattcttggtactaatttaatgtttccatctgtcagaaagctacaacgtttttcctccttctttcttttttttaagaattattttaaaaagtcaatacggtgctataattagatttttatttttcctcttttttagtgtatatatatatttatataagtagagattaggaactaattgattgaaaattaaatatgctgtgacgctcaaaagatattaatcccgcgttggttttatgtatttaaaaaatgtattttttcttttttgatatttttaaataataaaatatttaaattatttacaaaatataacaaaatttgctcgtttacatttttattttgtgaaggacttatgcgatgtggttcgatctagaattcttgtattttcaaaatagatggagcttctttttggatgaattctctctaggcttctgaagtcaaaaattttcaacccaagaaaaaactagagtttccttgtggtatgaggtgtatgaaattgactcattgactcaattacatggacttttatcatatttaactcagctaaattaagtttattttttggaattaatctaagtaaataatatttaattgaaccaaaatatttaatttgatcagtcataataaagacatgtgacatcattggaatcagtcaatttgtgtttaaatttaatttgggatacatgtcaacttttagttaatctcaaatgcaatttgtgattagttacaaaatttcttattcaacatacttcaaatctaaatttggtaaattatgttttttttaaagaaattagatcaacacaaaaatataatatgttttgtgaaaatgaaaattttggtttaatgggaggagacaaatttgaacgacaaattttcttagtaacttacgatatgatcactaactaatttattataggttgggggtttgaacctctctcaactttgtgctcattatataatatatctttaaaagaccgaccttgcattaatgttgttggttagtctagtggtagaatcgtcattctctagctctctctaaattgttccagcttcagtttttatatacttttttatattatttttactgaactataaaaaattactgtcgacaaaatttatgcttttatcacttaacataataattgaactatgtttgctttgttttttcttttttaacgtatactatctcaaagttttggataatgtacgtgtttgaaattttgtaacaaacaaggatacataaatacgtaattgttgttaattatttcaaaatgtaaatagatgatatgatgtaggggttgctaatattattgtctaattatttttgtaaagaataaaataaaataaaaatccatatgatgcctaaggacgtgtttagtattcaatatggcgattattatgtctttttttcaaatggattactttttggatgatatgatatcttttattttaattaaaactttttgatgacttttaaaatttaaagggcataaaatatagctttcttttgtacatatatatggttgtgcttggattttgtatctgcttgtctttgtcccgagtttctcgtcggtttgaccttcgatctactttttttgatatatatgttccaatatcgatttctaagctaacatatgtaaaagatgattgcactcgtgtggtgctgagactatcacacttgatacttgatataagattgtattattcatctgaaccaataaaaattaagatggatattactcactcttctcttttaggatcatcaaacaaggctctttttttgacaatcacctgaagtcacgagtaactctacagtgttatagttgtttatagtatttgagactggaaatatcaaattcagccaattcaattaaattttataattgatatacattcatatgctcgaaaagttggcttccaaccgtccaacgctctaactttgggacatgtgttacaaaacacataggtttaaaaaaatacctaaaaacataaaaaatacaactttgatcatcctttagttcaattttattcccaatttgatacttttgaattccctaactagagaattgtaattgtgattgaacgtttttatagtcaaaatttggatttgatagcttataggaccccatgtgccaaacaaaataaaaattgcatatatatatatagagagagagggggagttttatattttaaaaaacaaaattagaaaaaggcaattaattttgttctttaaacgacatggtttctcacgtgttaaacgtcattgttatcaactgcgtcttgtcgctgaccaattcattgacagcacagcaagcaaaaaagaaaaaagaaaaaaaaaagtcaaaaagttaatgtggttaaagaaagcgctttttgtgataactcaaaaaaacaaaccaaccaatggaagccctccaggtcactctcatgtggctcccgtctccacgtcatcaatgaacacggacgtgtttacagctcaacaaacaacaactattcctctgacgtggcaatttcttattaatgaatttccaactctgccctcctattacattttcttaaggtcggtgaatgccgtacaatggaagctcctagacatactttctagttgtgatatatatatatatatattattcatccatcatatattaaactccaatgtatgtttgtggattttacaaaagttaatattatttggtagatgttgagatttcttttttttccaatgtctagtctttttccaagtttggagaaagttttttatgatgttgggagttatttaatttcctagtggggccacgtagttaaataaatataggttaaattttacgaaatatccatatatcgcgtgtgtggtatgatttcagttacgtcactattttgaaaacatagttttcgtgttcttattaatgcttatagttgtaaataacataattaaaagtatcatttgttaaaattgatgtcacaccgtatgtacataatttatttattgattgatgttataaggggcgttggagatatcgttggaaaaaaatgatttgtaaggatgatcttaattttctataattgactcacgtatattatattgtatacgtttttcaaaatttacacaccaatcatctcactttcgttttcatttttcattttagtggaaaacaattcaataaaaaaaaattctgacaactttttaaaatttaaggcacagttgaatcaatccaaccgttcaagatttaaagaagaaaaaaactaatttggttgctccactttttgtttttgttcgttttggtccattaattctaaaaatgtttaatttatttgttatactttcaaatcttcacaactttaccgtattgatcccttaaaaatgaagtaaaaacaataatgaacgaactaagacaatcaccatttgaaagtttaaggaccaaatgaaccaaagttaaaagtatagaaacaaaaataaacatcgctaaataaaccaaataaaactagaattacttaattgaaacaaaataatatgaaatggatcaaatctttagactttagtgtatgggaaagttctatgaaaatgaccaccgactatcgagagaccaattttggggccaagtcaatgattggtaatttcaacctacatttatgatgtatgacaatgacaatagcttaggtcactttgaaaatgactataagattttctagttagagatatacacttgatattagacttggtcgttgtaataaaaactatgtgtcacggatgatatatgctaagtacatgttttagtctttaatgtttgcgtatatttctttacgtaatttaatcttcgttaattatattttttaaactatgttttaatcttttaattcttttgttgtgaaattgacaaataaagagaaacacgacaatgtagatggtaaacaatgaagtttgtgtagtttattgacaatatgttggcaatgtttacgagtagagagataattgttcaaattacagaacaataggtgacaatacgtgagtttttgttttaatttacttttgaaactttattttgattttcaaaacttaaagaagttgatactattattgttttgagctatgaagatgtgtgatcgaacctttcacacgtttagaatgaaagagcatgtcaattaattttgagctaaacttgtttaaaaaaattgaccttttgtctttgttttaagttttaacaaattaatgatgtcattgcgtaattttaagtcagttaggtatgaaaagccaccatcgaagaaagaaaatttcaagaagaaaagcaatgtagtaaatcacaaataattgtttttctttcccataggttataactataaaaaaaaaacttcttttttagtataataacggtaaagaaggatgatcaaaccttctaactcagtcaattgcaaatatgataaattcactttgacaaactaaaataaatttgaagatttatgaaacaaaatgtacattttaaaagtttatattttcacacaatgttagcttccttttaaaaaaaaattaaattttaaaagttcagagaacaaaacatacatttcaactttgctaacttcaatatagaacttatataaaatcgtgccacatagggttcaaaagaactataggattttaaaatgaaaacatatattttaattgagttttagaactaagttaatatatttatttataaagaataacacttcagtaaaattaagaacaacacagacatagttcaataacataaaactagctagaggatccccgggtaccatggatatctagaattcgcggccgcaagcttgatcccccgggctgcaggaattcgatatcaagctagagatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattagatgataatcatcgcaagaccggcaacaggattcaatcttaagaaactttattgccaaatgtttgaacgatctgcaggtcgatcctagacgcgtgagatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggatcgacctgcaggtctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacggatctctagcttatcgataccgtcggctattggtaataggacactgggattcgtcttggacaactttccttctcatctaagcgtagacaaccctcaactggaaacgggccggactccagggcgtgtgccaggtgcccacggaatagttttggccagacccttgaaaatccgattcagtacaatcgattgccctcatttttacgttggcatatatcctgccaaacagccaacaacgcgcgtgcggtgaataggaaagcgtttgagttgcttgctcatatcgtgacggttgacagcacaggttgaccgcttgatgattcgtacgagccgccaaacattggctgtcgtaatgatataccatgtcagaacagcaatccgatggggcggaaagcattatcttaatgcacacggaaatggcgcgtcggtgggtggaatacaccgacatagaggccgtaagttctgcatggtcatcgtcggaaaggtggcagcaggcgcacggctgtggcctcttgctctttcagcgtgaaatgcgtgttgaaagaataatcgaagagagcgtccgctcgacaccttcaattatgccgatttgatcgatgaactgatcgagctctgaaatcgaaggggcttcgataatcgcaatcaaatcaaaagtgccactcacagaatgaagagcgataacggccgtgaccttcccaagggaggccgtcacctgtgaaagcgccttcgtaatggtgatcagaatatgggctcgaaccaagctcgagacctcgagggggggcccggtacccaattcgccctatagtgagtcgtattacaattcactggccgtcgttttac

SEQ ID NO: 10 - DNA, 7621 bp, Cucumis melo
ttcttatcctctctctctctcttgccttttatagtagtggggttgggttagatttttggccatggtggaaagtatgaataagaaaagtagggagtttgagttattttgttgtttgatatgaagttggacaaagtgaatatacaatatattgctatggagaaaaagaattgaggttgtaatgttggaaaaaggatcttatctcattgttatgattatctaggccccaaataaaataatggagcatcttgaaagaagaaaagcactttttcttattttttggctttttaactttgctaattattttaggaaaaatatgagtcagccaaccccatttcccaaaatccaattctttttaataagtaatttctgtaagaggatgattttaattatagcatttattttctttaaaagaaaaggaaaagtattttcgtcaatcattgactgaattcataatgctataagttgctttacatgatgatgaatttgaataccaatctaaaattatggttctctgctttgaatatttaagtaatagattagattttaaacctttttccatgaaatgttatttactgatgttactaattgaatatattttgatcctcacataattcagtaagaccaaaaatgggtgattagaaaatattatgtaataaaactaaaccaattaagttgtggaagacagttcaatttaaactatggaaatagactccacaaataatagttataggctaataaataagaaatctttaaagaatttgaaaagtaagaatttaacttattgcctaaagtcttatcaaaccttatctcaaaacttacgtacggaataacataaatcatttcactttggttggaaagctgatattaaaattgggtatgtatgtttatttttatttttattattgctattaatattactattattattactattattattgttgttggttggacctaaattcaaaaatacccctgaattaataggatattttaaaataattggtgaacttagagaagtgttatttattattattattattatcattttaatttatgttgatttctactgtatataatctaaaatttgggaaaatattaatttaaactttgaattaaatgttttatcagcacattaactattgcatttcatttgaaaaaattgtgtaaattttataataatttaatgaactttcataaataaatcaattttggcttttagttataatcgtaagaatcgttacgtccattttgtaaaattgacttttgaacaagtctacacatgtatgaaagagaagcgatagtacgactatttttaaagttgatcataattaatggtctaaaattcattgacttatgaaaatttatgagtttaattaactcaaaccataaatttcatgcggagaaatgtataaattgctgaactttcaaataaaatcagataggagttataaatcgtaaagagagtcgtggatgatattaggtgtcgttttcaactttattgttattattaagattaacatgttggtatttatgaaacatgagcactgaggtgtttaggtagaaatttaaatgaagaattaaggatggaggcagcaattagaaaagaaagattgaaaacacagagagacagagaagaaatatcgcaattagttttcacattaaaactagacattaccaataaatcataattaatatcacttatctttttctttcctaacttttgtgaatattcgaatcaatttacatacagtatgattaattttacgggactatttacatgatcttatgattttttttttcttttaacgctcatgagtattttagttaacttacattcacttcgactaatcttcgagaaccatgtacctgattctccaacgtttaaacctaaaccttaataaaataatttatatatatatacagtgttttatgtgtggaattgtgtgtattatggaggagtagggacaatggtgagcgttggcaaattaaaaaaaaaagaagagtagtggaattaggcgacaaggaaaccaagacgtgaccaccaaagcaaatgttggccttttcacacttttcatttgatcatttccaaagaactatcaaaaaaattttcttatcttctcttttggcaaatttggcaaatacctacggaattaccattacggtctcctaatacattctccaatgttagtcacttactttgcccatttcttcccctttttctcaactctcatctctttttttcacacttgcacacttacttctagtttatgttctctccttttgcacgtgaatttcaatcgacacttctaatattatgtctccgatccaataaacgtgttggaatttggaagttaaaccgaattagtgtatcatctagtaagatttgaaatagtatgtatagcttttctttctaaaaaattttaagaacgacaagtcaaacctcgttaaaaccaaaaacgtataacaaagaaaaatcatcatttaagacttggtgacatctcattagggtaaacacttttccaacatgcatcacgaccttcctcctcaaacataagaaataagaaaaacaaaacaaaacaaaattaacacagcaaaataaaaaataaagttacaaaattacaagaacagtagatgaatttgttttatctacgtgaagagggttatgatgtttttttaatatatatatatatatagtcttcttcacactattcaatgagtcatttgttctttcaaatcccaaaaattgtgatgtctctctcaccacacacacacaccctttgtttccccctctcttttctttccccttctcattctttagtcattatagcaagtgatcttattcttggctttgtcctttatgttaggagtttctctttaattattttatttttatttctccaatctctctctatttctccctatattataagggtgttgttgtaaacagtcctctcccatcaaaaaaaaaaaaaaaaaaaaaaaggaaaaaagaaaaaaagatgggtgatcctaatctctccatcaataataataacaatacttgttttagtttatccccttttcaacattactcttcttcttcttcttcttcttcctcttcttataactctcattatcatcatgatcatctcttctcactttcttactccaatgataataacagcaataccctaaaaaacaacatgactacctctcattataatccttcttcttcttctcaagttcttcttcctcttttgagtttaagtcctgcaagagtagaacaagatcatcatcatcatcatcatcaaaatattattgatcatgatcataatattattgattatgatcaaaatgatgttactgttgctttgcatttagggcttccaactccttcctcttcttctaataataattctgatttgattttgaggctttcttcaactgagatctcagatcaagaagatcacacccatcaattgcaagaactttcatctaataattctattgctagtaatagtaatggagttaataagggtcaatattggattccaactcctactcagattctcattggtccaactcaattctcttgccctctttgcttcaagactttcaatagatacaacaacatgcaggtacttttttttttttcctttcaatttttatgtgtttattttgtcacttttttactctttttcttttctttcatgatataattttactgaaaaagtggaaaaaaaattcacgataattttggttaatttatttttttagtaccaatatatttgaaccattgatttcaaagtcctttaacacatacaaatgtgatttgagctatatatatatatatatatatatatatatatatatatatatactctcttctgtttttttagggttttggttgttctttttatacgtatatatggtcgtcaacacgtacaaatgtcaatgtcaatgttttcttcttctacttctttttttttttttttttttgaattttttcatgatgatattatatatttttggtgcatggtttaaaattattgtagtttttggggattttcaaaagatcagaacaaatcttcatctctggtgtgtgtatttcagaatgtaaaatccctttctttatgtgattgaacaacacacagcagccagtttgctgtatatttgacacattttgaacttcgtgtgttatttaatcatctttggcattcgtgtgaaatgaaatctaactctaattttagttaactcaaacacaaatacatatatatttaaaaaaaaaatccataacctgagtcagtagttgcacctgatgaaatttcattatatattatttcttctaattatatattcatgtttgttaatttccaacaagtctaatgcaaatttggagatttatattcttggtatatttgtacttcaacactaaaaggaatcccaagtttttgagaacaacaaaagctttttttttttttttttttttttttttttttttttttttgaactctttgcttttccaatttctccctttgaccaccataataattattgactcaggagggtttgatatcatttattaagttgaaaactcctgaatcttttgcctaaaaagaactgatagagaaatgtgatgatatatctacaagaaaaatttgtaacaaagtaactcaacttcttcatcagatttgccccctcattgtgcaacaactttattataatataacatgtttaatttcaaaccctttactaattcatacacatgttttgaaaaaatatcataattttttaaattaattaggatcattatatagtattatggatctgtatggaaaaccaagaatgtggttgatagtgatcgctcatcatcgtttagggtgtggtttcttttttaagcgtacttgtaattttgtcgttcactttctggtgtgtatgttgcggtggattcttttaatccataggttcactttctggtgcatatcgaaaactaactctcatgtgtcattaagtatataaatttctataaaaaatacttaaggatatgagggttagtggtgttgcctattggaagattgattatacccttgttggatatcctttggtcttatggaggattgcattgtgttgttgtttcaaaaatgctttttattattaaaaagaattcaaaatttaattgaaaatcataagtttttatcgtttaaaagaaaatacctgagaatacgaaggttggtcgtaaatataaatacgaatatctcaactaggtagacacgtacattattcattagtatactctcatcgtactttgtcaactatagattagagcattgacctatattcgtgttagcacgtgttcaacattgcatgcattgaaatggtagtctaagcttttttattgttacggtttcattaattttatgattaattaactgttgagttatattattatgctgacatgtttagcattcggaataacagatgcatatgtgggggcatggatcacaatacaggaaagggccacaatcactaagaggaacacaaccaacagcaatgttgaggctgccatgttactgttgtgcaataggatgcagaaacaacatagaccatccaagatcaaagccattaaaagacttcagaacacttcaaacacattacaaaagaaaacatgggatgaagccatttacatgtagaaaatgtgggaaagcttttgctgttagaggagactggagaacacatgagaagaattgtgggaaactttggcattgcacatgtggctctgattttaagcataaaaggtctcttaaagatcatatcaaagcctttggacttggccatgctgcttatgggattgatgatcatcataatcatcatcattcttttgataatgaagatgatgacccagcttctgatattgaaacatgactatcataatatatataatataattatgaacattattattgatatatccccaatgttctttttttttcccccctttttaacaaatttcataccttattttagctgtttgtactgttaaattggagttgttttcaatcattattgttcttatctttaatgagttcatgctagctaatttaatatcaagctatatactcaaattaaaattgatattcattgttcatttttattttatttacatttagatttggttgatagaagttctaatgacgtcttttgcctatcaacaaaggtcttttcttgactttttgtgttaacaaagatctagaccacaatcgtggtagtgatggagttgagatataatgagagtgtgaaaaaatatgtcaacctatttagaatatattagtgtgtctactaaccacctatctatcatatttgctttctaaaaatagaattacaacttttcattgaaacccataattttgaacttcttaccatataggtgaggaatgtagtttattacttccatataattacaaaatgccaatacaataatatggtttaaattatgataggctatatgtgtatcatttaggtataagaaatacaattagatgtgtaatataatgcttaattacaagattgtttgatttgaaatcaaactaatttggttgaatgattattggaaactatttggttgaataataatggtttccaaattaaaataaaagggcaaagcagcttaagcttaattaacacgcaactgaaacgattttacacaacaacacaacccctccatcttaaattctatacttgttgagatgtcccatcatgaccctcatcacgattcaaaattacacccaaaatgcttatattcctcttgtacttagtatccattaataaataaataacatccgtattatatttatactccatatcaaaacgtctaattctcatcattccgtctcttatttgtgtgccggcgcgtcctatctattatacctaagatttaaaagcaaagaatatgtatacgtatatcaaataaaatagagcaacaaaagaattcaaaaaaaccaaaagaaagtgataaaaagaacaacgaagaggagttttcaatctcacggttgaacatgttaatagaaaaattagggtttttaatgtatcctaactcatcgacttaatattttaggccaattaatcaaaataacatcatttttcttttaatgtaattaattattagatgaatggaagattgggaatatattaaatagaagagaggtttgaaggagtgagatggtaaaaaaagaagggtgtgtagggattttcattgagatgtcatctcaaaggggaagaaggggtttttaggtccagaaagaggactgttacaatagaaaaaatggaacagttatagtagttattagggggaagaaaaagggatttttgaaaagagtagaaggtactccaaatgaatggctttgatttaacacaacactcaaaataaccaataaataaataatgctattgttttcttttcttcttaaatctataatatatatatatatatatatatatatatatagtctcttacaatttcccagtgttctgtcttgcagcactaataacaatctgtctgccattcagacatgatggatccatgtaatttcacctctttttctttttcttctctcttttcttttcttttcttttctttttaaggttt

SEQ ID NO: 11 - DNA, 1062 bp, Cucumis melo
atgggtgatcctaatctctccatcaataataataacaatacttgttttagtttatccccttttcaacattactcttcttcttcttcttcttcttcctcttcttataactctcattatcatcatgatcatctcttctcactttcttactccaatgataataacagcaataccctaaaaaacaacatgactacctctcattataatccttcttcttcttctcaagttcttcttcctcttttgagtttaagtcctgcaagagtagaacaagatcatcatcatcatcatcatcaaaatattattgatcatgatcataatattattgattatgatcaaaatgatgttactgttgctttgcatttagggcttccaactccttcctcttcttctaataataattctgatttgattttgaggctttcttcaactgagatctcagatcaagaagatcacacccatcaattgcaagaactttcatctaataattctattgctagtaatagtaatggagttaataagggtcaatattggattccaactcctactcagattctcattggtccaactcaattctcttgccctctttgcttcaagactttcaatagatacaacaacatgcagatgcatatgtgggggcatggatcacaatacaggaaagggccacaatcactaagaggaacacaaccaacagcaatgttgaggctgccatgttactgttgtgcaataggatgcagaaacaacatagaccatccaagatcaaagccattaaaagacttcagaacacttcaaacacattacaaaagaaaacatgggatgaagccatttacatgtagaaaatgtgggaaagcttttgctgttagaggagactggagaacacatgagaagaattgtgggaaactttggcattgcacatgtggctctgattttaagcataaaaggtctcttaaagatcatatcaaagcctttggacttggccatgctgcttatgggattgatgatcatcataatcatcatcattcttttgataatgaagatgatgacccagcttctgatattgaaacatga

SEQ ID NO: 12 - protein, 353 aa, Cucumis melo
MGDPNLSINNNNNTCFSLSPFQHYSSSSSSSSSSYNSHYHHDHLFSLSYSNDNNSNTLKNNMTTSHYNPSSSSQVLLPLLSLSPARVEQDHHHHHHQNIIDHDHNIIDYDQNDVTVALHLGLPTPSSSSNNNSDLILRLSSTEISDQEDHTHQLQELSSNNSIASNSNGVNKGQYWIPTPTQILIGPTQFSCPLCFKTFNRYNNMQMHMWGHGSQYRKGPQSLRGTQPTAMLRLPCYCCAIGCRNNIDHPRSKPLKDFRTLQTHYKRKHGMKPFTCRKCGKAFAVRGDWRTHEKNCGKLWHCTCGSDFKHKRSLKDHIKAFGLGHAAYGIDDHHNHHHSFDNEDDDPASDIET

SEQ ID NO: 13 - DNA, 2999 bp, Cucumis melo
ttcttatcctctctctctctcttgccttttatagtagtggggttgggttagatttttggccatggtggaaagtatgaataagaaaagtagggagtttgagttattttgttgtttgatatgaagttggacaaagtgaatatacaatatattgctatggagaaaaagaattgaggttgtaatgttggaaaaaggatcttatctcattgttatgattatctaggccccaaataaaataatggagcatcttgaaagaagaaaagcactttttcttattttttggctttttaactttgctaattattttaggaaaaatatgagtcagccaaccccatttcccaaaatccaattctttttaataagtaatttctgtaagaggatgattttaattatagcatttattttctttaaaagaaaaggaaaagtattttcgtcaatcattgactgaattcataatgctataagttgctttacatgatgatgaatttgaataccaatctaaaattatggttctctgctttgaatatttaagtaatagattagattttaaacctttttccatgaaatgttatttactgatgttactaattgaatatattttgatcctcacataattcagtaagaccaaaaatgggtgattagaaaatattatgtaataaaactaaaccaattaagttgtggaagacagttcaatttaaactatggaaatagactccacaaataatagttataggctaataaataagaaatctttaaagaatttgaaaagtaagaatttaacttattgcctaaagtcttatcaaaccttatctcaaaacttacgtacggaataacataaatcatttcactttggttggaaagctgatattaaaattgggtatgtatgtttatttttatttttattattgctattaatattactattattattactattattattgttgttggttggacctaaattcaaaaatacccctgaattaataggatattttaaaataattggtgaacttagagaagtgttatttattattattattattatcattttaatttatgttgatttctactgtatataatctaaaatttgggaaaatattaatttaaactttgaattaaatgttttatcagcacattaactattgcatttcatttgaaaaaattgtgtaaattttataataatttaatgaactttcataaataaatcaattttggcttttagttataatcgtaagaatcgttacgtccattttgtaaaattgacttttgaacaagtctacacatgtatgaaagagaagcgatagtacgactatttttaaagttgatcataattaatggtctaaaattcattgacttatgaaaatttatgagtttaattaactcaaaccataaatttcatgcggagaaatgtataaattgctgaactttcaaataaaatcagataggagttataaatcgtaaagagagtcgtggatgatattaggtgtcgttttcaactttattgttattattaagattaacatgttggtatttatgaaacatgagcactgaggtgtttaggtagaaatttaaatgaagaattaaggatggaggcagcaattagaaaagaaagattgaaaacacagagagacagagaagaaatatcgcaattagttttcacattaaaactagacattaccaataaatcataattaatatcacttatctttttctttcctaacttttgtgaatattcgaatcaatttacatacagtatgattaattttacgggactatttacatgatcttatgattttttttttcttttaacgctcatgagtattttagttaacttacattcacttcgactaatcttcgagaaccatgtacctgattctccaacgtttaaacctaaaccttaataaaataatttatatatatatacagtgttttatgtgtggaattgtgtgtattatggaggagtagggacaatggtgagcgttggcaaattaaaaaaaaaagaagagtagtggaattaggcgacaaggaaaccaagacgtgaccaccaaagcaaatgttggccttttcacacttttcatttgatcatttccaaagaactatcaaaaaaattttcttatcttctcttttggcaaatttggcaaatacctacggaattaccattacggtctcctaatacattctccaatgttagtcacttactttgcccatttcttcccctttttctcaactctcatctctttttttcacacttgcacacttacttctagtttatgttctctccttttgcacgtgaatttcaatcgacacttctaatattatgtctccgatccaataaacgtgttggaatttggaagttaaaccgaattagtgtatcatctagtaagatttgaaatagtatgtatagcttttctttctaaaaaattttaagaacgacaagtcaaacctcgttaaaaccaaaaacgtataacaaagaaaaatcatcatttaagacttggtgacatctcattagggtaaacacttttccaacatgcatcacgaccttcctcctcaaacataagaaataagaaaaacaaaacaaaacaaaattaacacagcaaaataaaaaataaagttacaaaattacaagaacagtagatgaatttgttttatctacgtgaagagggttatgatgtttttttaatatatatatatatatagtcttcttcacactattcaatgagtcatttgttctttcaaatcccaaaaattgtgatgtctctctcaccacacacacacaccctttgtttccccctctcttttctttccccttctcattctttagtcattatagcaagtgatcttattcttggctttgtcctttatgttaggagtttctctttaattattttatttttatttctccaatctctctctatttctccctatattataagggtgttgttgtaaacagtcctctcccatcaaaaaaaaaaaaaaaaaaaaaaaggaaaaaagaaaaaaag

SEQ ID NO: 14 - DNA, 8255 bp, Cucumis melo
attttgagtaggggtgtaattgggtcgggtcgggtcgggtcttttttttttttttttttttttttttttttttttttttttcctccttccttagcaaggaggagattttcacaagggggagaaaatgttccggggagtgcatcatttgaacaatacttgaatagaaatgtagatcatcataactttttgtaagtgcattttccctatgttagtacatactcatcaagaagatgttaccatgaaatcaattctatatccatagttgtcattcattttggtccttacttgtcatcaaaccaagcataagatattttgtttacaaaaactgcacataactgctgtcaattaaatattaaccgaaaataggaaacaagaaatgagaccaaaagcacggatataccgcactacacttgctacatcaaggtcgtctttacttattacaaagtgaaggattaaaagtttcatttcacagtataactgattagggaagggagctaattcagaaaactgacggtatgtctgctaaaatttttgaagttctctaagccattggaaagatctcctcaacagatgaacgtaatggtactacataaaatcttaaattaaactgccttcacctttatgccacagatacagctgcaactacaatacatcagtaaagcccattatcaactatgggatgaagtcttgtgtagcaattattctgtaggaaaacttatcttataagctatgacactaacagcacaagtataggcaattcaagtgccatcaagaggaaagagcagacggatccaagcagaatccagagccacagttatgtgcacgagaaagacaggaaaggcacaaagaaaaatgaaaaatactaaaatgttatgttcacactctacttgtaagcatgctgcagggaatctatagtatttatttaatattcaagaatgctataacaatagctggagacctgcaataaataagtgtaacctattagagaggatggttaaacaaatctaaacttctgtcatatgggaaaggataacaaatacaactaattcaaacctattttttcctgacatgcagtgcaccagaacccgagccttgtctctcacattgctctgaaaatcacaagcaatatcaggaaagttagacttttggaataagtgaatgatagaaagctaatgacacgttcttaaccaaaagtcatttgttcagtgtcatcttatagttcatttgggagaatataaagaaaggccacctagaaattcaactgcgtcatcaaaaggtaaagtcttatcatattggagacagtgatacgtgaaggagttcttgtagagattttggcaagcaggcacagtctgcaaaaatagtgtggagaaaaccaaatgtcagtcatttattttcagatcacccaatacattttagaggcaagtgatgatgcatgacttctacaacatccgagagagataaccatgatatgtattctacaatgaaactgctttttagtataaccatcaacccaataaaacttatttttatatctacgaaagccaacaacttgatatcattgcattcaatatagattatacatcataatagattagaaaaatagttataagcataaaatactcagagtccaaggtaacctgagtctccaggcaattcattctggcatttgccatattaccaaaaccttaataggctagtagtacagtccatttacggcaattatgtgctggtgctgttatttgttagtatcagagctttcaatttataaataaaatccataagcaggctaaaagcaagattccaagatgtaacgaagccgtaagcataaatcagaatagtagtcacacctgggcagcatacgtccgttggaatggaaattacattggcctcaaatattcaagtagtgaacttaaggactttgacaattacgaataaagaagcagaacctgacaaaatgtgatccaggatctgtaaatatacctttgaaataccatatccctattgcttttgcttcctgtccccaactttgaacccaattcattctcttgggtattgctagatttagcagaaacaacatcagctacttgattagccaccggagccttgccaacctcgcccgcattcgctaaatccatgtttcagatagattgaaagtagagagtataaaagataacaaaggaatttggaaaacactggggaacttttacaaacgcatggaagacaataaaaaccaaaccaataatcagtaagttaccggttcaacgaaaccaccatgaaatcaactaaaacaaaaaacaaaacaaacaaacaaccaataatctctcagcccttcttttgtttcttttatataaagaaggttaaccaaggaacctcatgaacaaagtttacaattgggaaggtatgaattaatgaacatcttcacgagaccaccttgttcgtataacccaaagtgcttccatcattgagcttcaggactctaaaaaaacaagattcgatatttaataagttaataattttaaaaaaatgtaaataaagatttgagagtaaggatttaaaccatagagtcattattcaaattctcaaatgcagcttccatctcctttcctatgtttatgaattctagatcaaaatgacaaacagttagcttatatgggataattgaaaattttaaatgcgtacttcaaaaagaatattaccttcatcaatttcttcagccccgtcaatttcttcagtcatgtcatccaaaggtttagactgaatccaattctgagcacaaatgagtgcctctgcagtttgaggagttaaagaacttcgaaaagaatctaacacccgtcctccagtgctaaaggcggactcagaaggcacagttgatataggaatactgtagatgtccctagctacttggctaatgatcttaaatcgagaggcattcaccttccaccaagttagcaaatctaaatattcatcgcccatacaatctatacgagcctcatccagataacgagtcacctctgttttagcatcatctagacatgttttgttactttgtttaaatctatcatgaacagtagcacgtgccttgtaagatccactagatgagatagaaggtatttcactttgactttgaaagccaaatccttcgataggtgtacatgattgtgtttgtgaatatttttcttttgacattctcatataataatcatcacacaatcgacgaaatgcttcttcaaccttatttgtccatatttttgcacaatcttcctccaaaaattcattaaaacaataattcacataagctagcttgtatctagggtcaagaactacagaaacatacaataataaattggtcttctcacttgtagttataccccaatacttgttgaattttgtctgcatgcttaatgtcatttgactcaataatgcattctcatacgatgagtattcacgaattatttcttggatcaaacaaagttcatgaaaaaatatatttgaagtcacagacatagatgcagaaaacttcattgttacctctgaaaaagtctttaggaactttacaaacacttttgcattatcccaatcttcagtagtaggaatatcatcctttggcaaataactagggtcatgctcctccaatctttcaaaagtcttttgacacttaattgctccatccaacatagtaaaagtagaattccatcgtgtcggaacatccattgtaagacaattttttgttgacatcttatcttctttagcaaaatctttaaatatttgcaatctagcaggagatgacctaacatacttcacagcatttctgattcgaatgatagacacatgcaaatcttttaaggcatcactaacaattaaattaagaatatgagcacaacatctaatgtgaataaattcaccatccaacaccaacccatttctgcctttaaactttttaaccaagtaggcaatggctacatcatttgaactcgcattatcaaccgttacagtaaagagcctatcaataccccaaccttctaagcacttttcaatggctctacctatggtatctcctttatgattagctacttgacaaaagttcaaaattcttttgtgcaagttccaatcatcatcaatgaaatgagccgttataaccatataattaatattttgcacagaagtccacgtatccgttgttaaacaaactctttggccacttcgagttaatgcattttttaacttttttttctccttcatatacatttgaaaacaatcttttgcaacagttactcttgatggaatcacaaactttggatttaatgcccgacaaaattggtgaaacccttcactttctacgaacttaaatggcaattcatccaagataaccatcctagcaagcatttttctacaattttcttgagtgaatgatgcagtcatcaaattactttcagaatctccctctccttcaacattatcttccaatggatttacatacatcttacatttctctaaatgtctttttaaattagtggtaccatttcttttggaatcacaagcatatgaagccccacaatgtttacaagcagccctaggatatttaggatcacatccttctacttttataaaatgttcccataccgatgatggtggtttaaccggttttctttttcctaacactggactagaacaactctgatttgaagtctcgtctaccgagaatgaagtcatcctaataatcatcaaacaaaacacataaaaaaaatatcaatttttatggcttattaacttcatgcacagcagactcaacgactaaacaactccacccctaaaaagatatatggactgaaataatattttaaatatccctaaactatgtaaactaggagaacctaactgtaatttaatttctttgatgcaactaacccaaaataaatttattacacaataatgcaagattagtacaattttttaggtcgagtttgattagatgttttcaagttcatgaacctgtttaagttttagcaacttaatgtgatcgaagactgttggtttctatcttcaagactttatccatataaggttaaacatatgacacactcccacccaatcaccactacatcttccactcatatttgataattttttttagacaatttgaacagaaaaaaattggtagctgttatttatcatatttgataaacatttttttagataatttgaaccaataattgaagcataatcaagttggacaaagttcgagagctatcaaactagaaaagagagatagaaaactcgaaacaaaaccattttagataggattaattagcgaggttactagttgttaaatattaaataatcaaagcctaccttcgagatcgggatctcaactatgtgcaacatcgccaagacctaagatttggaaagactatttccaaaaaccaacgtattagtccttttgagtattctttcttcacttacattctttgtcatagaaagattcatagactttaaaatatttgagaaaggatgtcctaaacctctaaaggccaattaaataaaaatttctaaatataaaactctaattgatagaacctacaactattgtacataaaaatgaacaagaccgaatccaccctctagagaaccttatgtttgctggaaatgtatatggacttaaatgtaaagcaagatgaacataattcaaattaaaggtgcagcctaatagattcaaaatccactaatttgcttacttaatcataaaattcttctaaaaacatgcaagattggtcaatttaattcttagtccatgtactcaagaaagcttaagttggatacatgtcaattggaaaattaaggcattttgcataaaagaaaattaagaaaaaaagaggtacatttgaaactatttagaaaaattgggctattttttttttttaaacatcaaattcttaccaaacgagtggttagcaattacaatcctggttgatcccttctgttttgcaccacaagcaagagctttgccaactccaccagcaccaatgacaacaacgagctgagctttaccagctaagcatgaaccgactgtggagctgtcgttatgcaagcctgcaatataagagttgggtataaaccatcttggttgaaaataatatttatgaagaaacagaagaaaaatatgcatacaaaccaggtaatccaacttcaatggctgaaatagcaccataacaatctgtgttatagccacataatttttcatcagattgtcttctagttaagaacaaaaagttaacagctccaattggctgcaaaagtttaaaactctatcatccaagtattgtggaaactaagaggtctatatatatctgggggaaccttttgttttaggattcaagacatgtagatagataaagcgtgtagagccttacaattttgttgaaatatgatgaaaatttgtatattaatagctaaatgtgttttatgatgtgaaccgtaccatcctgatctacggctgaatgtagttttatactgctatatatctactaataatccatattactaacttttttataaaactaatccctatattatttactatccaatcaccgacatgtttcttcttggtctgtaggcttatgatattgatacactgtcagatgaacagaggtatgctatcaaggaccttgctgatctgggactagttaagcttcagcaggttttcggctagcctctgctgctttattgaatacgacctttgctcaaagttttactttattttaactttacctttttctttttctgttgttccttttgttgttgatttgtccagggtagaaaagagagttggttcatacctactaaattagccacaaatctttcaatgagtttggcagattcctcttcaaggaaacaagtgagtgtttctgaatgcaaattatttttagattggtatatttctattcagttccattttttttatgcttaccaagtttttagagggtgtttggctcgcgaagttggagataagagagtgtttgtgcccaaggaaaacatggatcccacaaactaaaaaaccaccaaaatttcatgcatcgttaactcttatgatccccatgacttcacaactccctagacatcataacttcactcttggcctcaaacccccttagtgttttgtgtgtttttgataattgcttttccataattattttttagttttatactgctatatatccgctccgcgatacttcttatgtaaattttatttgatagaatcttctttctgcaattaaataattcggttgagtgaagttttttttttctcttgggggggggggggtttaattctcagtttagaggctctatgaagttgcttgctcttcctaattttctttctgttacatgaccagggatttgttgttgtggagacaaatttcaggatgtatgcttattcttcttccaaactacattgtgaaatattacggtacatcgtgaggcagcagaagcaagctggttatcaaagttagtcaatctattggctagattaggcatttagtaagtgttgtgaaaatgagtcgatagcgtgaagtttgtgcagaggaaccccaaactgggaatgagtataaagacaattataggcaattgctgaggcactggacaaaatgagctgtaaactaaagactaagcttctcaaacagtctggtatttttcaatctacaaacaagaatttcatattatgtacacttaaaccagaacccataaacaagaacccagatggcaataaccccaaataaacagaaaaaaaacccaaaataatacaagctgtaatttaaaaattaaagagatgataataaggagaaagggacagacagaaagataaagcaatatatgggtcagtgtggaacttacaggcgatgacgatggagtggcgaagtgatgaagacgaagagatgccgacgccgatgaacacgaagagatagatgccgacgctgatgcagacgaagaggtagatgccgacgccaaacgatggaacaaaatggtttttgaagaggaggaagacgccagacgaaatgctgaagactgaagaagaatcgaggagaggaaatgaattatacctaatcgggtattttaaacccgacccgacccgaaccgaattaaatcgggtcggttcggtccagtaacattgttgttactaaccgatccaacccaaaccgaaccgaatcgggtcgggtcgggtcatcgggtcgggtcggtttttttcacccctaattttgagt

SEQ ID NO: 15 - DNA, 15447 bp, Cucumis melo
gctcggtacccggggatcttatctcattgttatgattatctaggccccaaataaaataatggagcatcttgaaagaagaaaagcactttttcttattttttggctttttaactttgctaattattttaggaaaaatatgagtcagccaaccccatttcccaaaatccaatttttttttaataagtaatttctgtaagaggatgattttaattatagcatttattttctttaaaagaaaaggaaaagtattttcgtcaatcattgactgaattcataatgctacaagttgctttacatgatgatgaatttgaataccaatctaaaattatggttctctgctttgaatatttaagtaatagattagatttttaacctttttccatgaaatgttatttactgatgttactaattgaatatattttgatcctcacataattcagtaagaccaaaaatgggtgattagaaaatattatgtaataaaactaaaccaattaagttgtggaagacagttcaatttaaactatggaaatagactccacaaataatagttataggctaataaataagaaatctttaaagaatttgaaaagtaagaatttaacttattgcctaaagtcttatcaaaccttatctcaaaacttacgtacggaataacataaatcatttcactttggttggaaagctgatattaaaattgggtatgtatgtttatttttatttttattattgctattaatattactattattattactattactattattattgttgttggttggacctaaattcaaaaatacccctgaattaataggatattttaaaataattggtgaacttagagaagtgttatttattattattattattatcattttaatttatgttgatttctactgtatataatttaaaatttgggaaaatattaatttaaactttgaattatatgttttatcagcacattaactattgcatttcatttgaaaaaattgtgtaaattttataataatttaatgaactttcataaataaatcaattttggcttttagttataattgtaagaatcgttacgtccattttgtaaaattgacttttgaacaagtctacacatgtatgaaagagaagcgatagtacgactatttttaaagttgatcataattaatggtctaaaattcattgacttatgaaaattcatgagtttaattaactcaaaccataaatttcatgcggagaaatgtataaattgctgaacttacaaataaaatcagataggagttataaatcgtaaagagagtcgtagatgatattaggtgtcgttttcaactttattgttattattaagattaacatgttggtatttatgaaacatgagcactgaggtgtttaggtagaaatttaaatgaagaattaaggatggaggcagcaattagaaaagaaagattgaaaacacagagagacagagaagaaatatcgcaattagttttcacattaaaactagacattaccaataaatcataattaatatcacttatctttttctgaatattcgaatcaatttacagtttgataaattttacgggactatttacatgatcttatgatttttttcttttaacgctcatgagtattttagttaacttacattcacttcgactaatcttcgaaaaccatgtacctgattctctaacgtttgaacctaaaccttaataaaataatttacatatatatatacagtgttttatgtgtggaattgtgtgtattatggaggagtagggacaatggtgagcgttggcaaattaaaaaaaaagaagagtagtggaattaggcgacaaggaaaccaagacgtgaccaccaaagcaaatgttggccttttcacacttttcatttgatcatttccaaagaactatcaaaattctttttcttatcttctcttttggcaaatttggcaaatacctacggaattaccattacggtctcctaatacattctccaatgttaatcacttactttgcccatttcttcccctttttctcaactctcatctctttttttcacacttgcacacttacttctagtttatgttctctccttttgcacgtgaatttcaatcgacacttctaatattatgtctccgatccaataaacgtgttggaatttggaagttaaaccgaattagtgtatcatctagtaagatttgaaatggtatgtgtagcttttctttctaaaaagttttaagaacgacaagtcaaacttcgttaaaaccaaaaacgtataacaaagaaaaatcatcatttaagacttggtgacatctcattagggtaaacacttttccaacatgcatcacgaccttcctcctcaaacataagaaataagaaaaacaaaacaaaacaaaattaacacagcaaaataaaaaataaagttacaaaattacaagaacagtagatgaatttgttttatctacgtgggaagagggttatgatgtttttttaatatatatatatatagtcttcttcacactattcaatgagtcatttgttctttcaaatcccaaaaattgtgatgtctctctcaccacacacacacaccctttgtttccccctctcttttctttccccttctcattctttagtcattatagcaagtgatcttattcttggctttgtcctttatgttaggagtttctctttaattattttatttttatttctccaatctctctctatttctccctatattataagggtgttgttgtaaacagtcctctcccatcaaaaaaaaaaaaaaaaaaaaaaaaaaaggaaaaaagaaaaaagatgggtgatcctaatctctccatcaataataataacaatacttgttttagtttatccccttttcaacattactcttcttcttcttcttcttcctcttcttataactctcattatcatcatgatcatctcttctcactttcttactccaatgataataacagcaataccctaaaaaacaacatgactacctctcattataatccttcttcttcttctcaagttcttcttcctcttttgagtttaagtcctgcaagagtagaacaagatcatcatcatcatcatcatcaaaatattattgatcatgatcataatattattgattatgatcaaaatgatgttactgttgctttgcatttagggcttccaactccttcctcttcttctaataataattctgatttgattttgaggctttcttcaactgagatctcagatcaagaagatcacacccatcaattgcaagaactttcatctaataattctattgctagtaatagtaatggagttaataagggtcaatattggattccaactcctactcagattctcattggtccaactcaattctcttgccctctttgcttcaagactttcaatagatacaacaacatgcaggtacttttttttttttcctttcaatttttatgtgtttattttgtcacttttttactctttttcttttctttcatgatataattttactgaaaaagtggaaaaaaaattcacgataattttggttaatttatttttttagtaccaatatatttgaaccattgatttcaaagtcctttaacacatacaaatgtgatttgagctatatatatatatatatatatatactctcttctgtttttttagggttttggttgttctttttatacgtatatatggtcgtcaacacgtacaaatgtcaatgtcaatgttttcttcttctactttttttttttttttttttttgaattttttcatgatgatattatatatttttggtgcatggtttaaaattattgtagtttttggggattttcaaaagatcagaacaaatcttcatctctggtgtgtgtatttcagaatgtaaaatccctttctttatgtgattgaacaacacacagcagccagtttgctgtatatttgacacattttgaacttcgtgtgttatttaatcatctttggcattcgtgtgaaatgaaatctaactctaattttagtttacatatatatttaaaaaaaaaatccataacctgagtcagtagttgcacctgatgaaatttcattatatattatttcttctaattatatattcatgtttgttaatttccaacaagtctaatgcaaatttggagatttatattcttggtatatttgtacttcaacactaaaaggaatcccaagtttttgagaacaacaaaagcttttttcttttttttttcttttttttttttttttgaactctttgcttttccaatttctccctttgaccaccataataattattgactcaggagggtttgatatcatttattaagttgaaaactcctgaatcttttgcctaaaaagaactgatagagaaatgtgatgatatatctacaagaaaaatttgtaacaaagtaactcaacttcttcatcagatttgccccctcattgtgcaacaactttattataatataacatgtttaatttcaaaccctttactaattcatacacatgttttgaaaaaatatcataattttttaaattaattaggatcattatatagtattatggatctgtatggaaaaccaagaatgtagttgatagtgatcgctcatcatcgtttagggtgtggtttcttttttaagcgtacttgtaattttgtcgttcactttctggtgtgtatgttgcggtagattcttttaatccataggttcactttctggtgcatatcgaaaactaactctcatgtgtcattaagtatataaatttctataaaaaatacttaaggatatgagggttagtggtgttgcctattggaagattgattatacccttgttggatatcctttggtcttatggaggattgcattgtgttgttgtttcaaaaatgctttttattattaaaaagaattcaaaatttaattgaaaatcataagttttaatcgtttaaaagaaaatacctgagaatacgaaggttggtcgtaaatataaatacgaatatctcaactaggtagacacgtacattattcattagtatactctcatcgtactttgtcaactatagattagagcattgacctatattcgtgttagcacgtgttcaacattgcatgcattgaaatggtagtctaagcttttttattgttacggtttcattaattttatgattaattaactgttgagttatattattatgctgacatgtttagcattcggaataacagatgcatatgtgggggcatggatcacaatacaggaaagggccacaatcactaagaggaacacaaccaacagcaatgttgaggctgccatgttactgttgtgcaataggatgcagaaacaacatagaccatccaagatcaaagccattaaaagacttcagaacacttcaaacacattacaaaagaaaacatgggatgaagccatttacatgtagaaaatgtgggaaagcttttgctgttagaggagactggagaacacatgagaagaattgtgggaaactttggcattgcacatgtggctctgattttaagcataaaaggtctcttaaagatcatatcaaagcctttggacttggccatgctgcttatgggattgatgatcatcataatcatcatcattcttttgataatgaagatgatgacccagcttctgatattgaaacatgactatcataatatatataatataattatgaacattattattgatatatccccaatgttctttttttttcccttttttaacaaatttcataccttattttagctgtttgtactgttaaatcggagttgttttcaatcattattgttcttatctttaatgagttcatgctagttaatttaatatcaagctatatactcaaattaaaattgatattcattgttcatttttattttattcacatttagatttggttgatagaagttctaatgacgtcttttgcctatcaacaaaggtcttttcttgactttttttgtgttaacaaagatctagaccacaatcgtggtagtgatggagttgagatataatgagtgtgaaaaaatatgttaacctatttagaatatattagtgtgtctactaaccacctatctatcatatttgctttctaaaaatagaattgcaacttttcattgaaacccataattttgaacttcttaccataggtgagaaatgtagtttattacttccatataattacaaaatgccaatacagtaatatggtttaaattatgataggctatatgtgtatcatttaggtataagaaatacaattagatgtgtaatataatgcttaattacaagattgtttgatttgaaatcaaactaatttggttgaatgattattggaaactatttggttgaataataatggtgtccaaattaaaataaaagggcaaagcagcttaagcttaattaacacgcaactgaaacgattttacacaacacaacccctccatcttaaattctatacttgttgagatgtcccatcatgaccctcatcatgattcaaaattacacccaaaatgcttatattcctcttgtacttagtatccattaataaataaataacatccatattatatttatactccatatcaaaacgtctaattctcatcattccgtctcttattcgtgtgcccgcgcgtcctatctattatacctaagatttaaaagcaaagaatatgtatacgtatatcaaataaaatagagcaacaaaggaattcaaaaaaaccaaaagaaagtgataaaaagaacaacgaagaggagttttcaatctcacggttgaacatgttaatagaaaaattagggtttttaatgtaccctaactcatcgacttaatattttaggccaattaatcaaaataacatcatttttcttttaatgtaattaattattagatgaatggaagattgggaatatattaaatagaagagaggtttgaagaagtgagatggtaaaaaaagaagggtgtgtagggattttcattgagatgtcatctcaaaggggaagaaggggtttttaggtccagaaagaggactgttacaatagaaaaaatggaacagttatagtagttattagggggaagaaaaagggatttttgaaaagagtagaaggtactccaaatgaatggctttgatttaacacaacactcaaaattaggggtgaaaaaaaccgacccgacccgatgacccgacccgacccgattcggttcggtttgggttggatcggttagtaacaacaatgttactggaccgaaccgacccgatttaattcggttcgggtcgggtcgggtttaaaatacccgattaggtataattcatttcctctcctcgattcttcttcagtcttcagcatttcgtctggcgtcttcctcctcttcaaaaaccattttgttccatcgtttggcgtcggcatctacctcttcgtctgcatcagcgtcggcatctatctcttcgtgttcatcggcgtcggcatctcttcgtcttcatcacttcgccactccatcgtcatcgcctgtaagttccacactgacccatatattgctttatctttctgtctgtccctttctccttattatcatctctttaatttttaaattacagcttgtattattttgggttttttttctgtttatttggggttattgccatctgggttcttgtttatgggttctggtttaagtgtacataatatgaaattcttgtttgtagattgaaaaataccagactgtttgagaagcttagtctttagtttacagctcattttgtccagtgcctcagcaattgcctataattgtctttatactcattcccagtttggggttcctctgcacaaacttcacgctatcgactcattttcacaacacttactaaatgcctaatctagccaatagattgactaactttgataaccagcttgcttctgctgcctcacgatgtaccgtaatatttcacaatgtagtttggaagaagaataagcatacatcctgaaatttgtctccacaacaacaaatccctggtcatgtaacagaaagaaaattaggaagagcaagcaacttcatagagcctctaaactgagaattaaacccccccccccccaagagaaaaaaaaaacttcactcaaccgaattatttaattgcagaaagaagattctatcaaataaaatttacataagaagtatcgcggagcggatatatagcagtataaaactaaaaaataattatggaaaagcaattatcaaaaacacacaaaacactaagggggtttgaggccaagagtgaagttatgatgtctagggagttgtgaagtcatggggatcataagagttaacgatgcatgaaattttggtggttttttagtttgtgggatccatgttttccttgggcacaaacactctcttatctccaacttcgcgagccaaacaccctctaaaaacttggtaagcataaaaaaaatggaactgaatagaaatataccaatctaaaaataatttgcattcagaaacactcacttgtttccttgaagaggaatctgccaaactcattgaaagatttgtggctaatttagtaggtatgaaccaactctcttttctaccctggacaaatcaacaacaaaaggaacaacagaaaaagaaaaaggtaaagttaaaataaagtaaaactttgagcaaaggtcgtattcaataaagcagcagaggctagccgaaaacctgctgaagcttaactagtcccagatcagcaaggtccttgatagcatacctctgttcatctgacagtgtatcaatatcataagcctacagaccaagaagaaacatgtcggtgattggatagtaaataatatagggattagttttataaaaaagttagtaatatggattattagtagatatatagcagtataaaactacattcagccgtagatcaggatggtacggttcacatcataaaacacatttagctattaatatacaaattttcatcatatttcaacaaaattgtaaggctctacacgctttatctatctacatgtcttgaatcctaaaacaaaaggttcccccagatatatatagacctcttagtttccacaatacttggatgatagagttttaaacttttgcagccaattggagctgttaactttttgttcttaactagaagacaatctgatgaaaaattatgtggctataacacagattgttatggtgctatttcagccattgaagttggattacctggtttgtatgcatatttttcttctgtttcttcataaatattattttcaaccaagatggtttatacccaactcttatattgcaggcttgcataacgacagctccacagtcggttcatgcttagctggtaaagctcagctcgttgttgtcattggtgctggtggagttggcaaagctcttgcttgtggtgcaaaacagaagggatcaaccaggattgtaattgctaaccactcgtttggtaagaatttgatgtttaaaaaaaaaaaatagcccaatttttctaaatagtttcaaatgtacctctttttttcttaattttcttttatgcaaaatgccttaattttccaattgacatgtatccaacttaagctttcttgagtacatggactaagaattaaattgaccaatcttgcatgtttttagaagaattttatgattaagtaagcaaattagtggattttgaatctattaggctgcacctttaatttgaattatgttcatcttgctttacatttaagtccatatacatttccagcaaacataaggttctctagagggtggattcggtcttgttcatttttatgtacaatagttgtaggttctatcaattagagttttatatttagaaatttttatttaattggcctttagaggtttaggacatcctttctcaaatattttaaagtctatgaatctttctatgacaaagaatgtaagtgaagaaagaatactcaaaaggactaatacgttggtttttggaaatagtctttccaaatcttaggtcttggcgatgttgcacatagttgagatcccgatctcgaaggtaggctttgattatttaatatttaacaactagtaacctcgctaattaatcctatctaaaatggttttgtttcgagttttctatctctcttttctagtttgatagctctcgaactttgtccaacttgattatgcttcaattattggttcaaattatctaaaaaaatgtttatcaaatatgataaataacagctaccaatttttttctgttcaaattgtctaaaaaaaattatcaaatatgagtggaagatgtagtggtgattgggtgggagtgtgtcatatgtttaaccttatatggataaagtcttgaagatagaaaccaacagtcttcgatcacattaagttgctaaaacttaaacaggttcatgaacttgaaaacatctaatcaaactcgacctaaaaaattgtactaatcttgcattattgtgtaataaatttattttgggttagttgcatcaaagaaattaaattacagttaggttctcctagtttacatagtttagggatatttaaaatattatttcagtccatatatctttttaggggtggagttgtttagtcgttgagtctgctgtgcatgaagttaataagccataaaaattgatattttttttatgtgttttgtttgatgattattaggatgacttcattctcggtagacgagacttcaaatcagagttgttctagtccagtgttaggaaaaagaaaaccggttaaaccaccatcatcggtatgggaacattttataaaagtagaaggatgtgatcctaaatatcctagggctgcttgtaaacattgtggggcttcatatgcttgtgattccaaaagaaatggtaccactaatttaaaaagacatttagagaaatgtaagatgtatgtaaatccattggaagataatgttgaaggagagggagattctgaaagtaatttgatgactgcatcattcactcaagaaaattgtagaaaaatgcttgctaggatggttatcttggatgaattgccatttaagttcgtagaaagtgaagggtttcaccaattttgtcgggcattaaatccaaagtttgtgattccatcaagagtaactgttgcaaaagattgttttcaaatgtatatgaaggagaaaaaaaagttaaaaaatgcattaactcgaagtggccaaagagtttgtttaacaacggatacgtggacttctgtgcaaaatattaattatatggttataacggctcatttcattgatgatgattggaacttgcacaaaagaattttgaacttttgtcaagtagctaatcataaaggagataccataggtagagccattgaaaagtgcttagaaggttggggtattgataggctctttactgtaacggttgataatgcgagttcaaatgatgtagccattgcctacttggttaaaaagtttaaaggcagaaatgggttggtgttggatggtgaatttattcacattagatgttgtgctcatattcttaatttaattgttagtgatgccttaaaagatttgcatgtgtctatcattcgaatcagaaatgctgtgaagtatgttaggtcatctcctgctagattgcaaatatttaaagattttgctaaagaagataagatgtcaacaaaaaattgtcttacaatggatgttccgacacgatggaattctacttttactatgttggatggagcaattaagtgtcaaaagacttttgaaagattggaggagcatgaccctagttatttgccaaaggatgatattcctactactgaagattgggataatgcaaaagtgtttgtaaagttcctaaagactttttcagaggtaacaatgaagttttctgcatctatgtctgtgacttcaaatatattttttcatgaactttgtttgatccaagaaataattcgtgaatactcatcgtatgagaatgcattattgagtcaaatgacattaagcatgcagacaaaattcaacaagtattggggtataactacaagtgagaagaccaatttattattgtatgtttctgtagttcttgaccctagatacaagctagcttatgtgaattattgttttaatgaatttttggaggaagattgtgcaaaaatatggacaaataaggttgaagaagcatttcgtcgattgtgtgatgattattatatgagaatgtcaaaagaaaaatattcacaaacacaatcatgtacacctatcgaaggatttggctttcaaagtcaaagtgaaataccttctatctcatctagtggatcttacaaggcacgtgctactgttcatgatagatttaaacaaagtaacaaaacatgtctagatgatgctaaaacagaggtgactcgttatctggatgaggctcgtatagattgtatgggcgatgaatatttagatttgctaacttggtggaaggtgaatgcctctcgatttaagatcattagccaagtagctagggacatctacagtattcctatatcaactgtgccttctgagtccgcctttagcactggaggacgggtgttagattcttttcgaagttctttaactcctcaaactgcagaggcactcatttgtgctcagaattggattcagtctaaacctttggatgacatgactgaagaaattgacggggctgaagaaattgatgaaggtaatattctttttgaagtacgcatttaaaattttcaattatcccatataagctaactgtttgtcattttgatctagaattcataaacataggaaaggagatggaagctgcatttgagaatttgaataatgactctatggtttaaatccttactctcaaatctttatttacatttttttaaaattattaacttattaaatatcgaatcttgtttttttagagtcctgaagctcaatgatggaagcactttgggttatacgaacaaggtggtctcgtgaagatgttcattaattcataccttcccaattgtaaactttgttcatgaggttccttggttaaccttctttatataaaagaaacaaaagaagggctgagagattattggttgtttgtttgttttgttttttgttttagttgatttcatggtggtttcgttgaaccggtaacttactgattattggtttggtttttattgtcttccatgcgtttgtaaaagttccccagtgttttccaaattcctttgttatcttttatactctctactttcaatctatctgaaacatggatttagcgaatgcgggcgaggttggcaaggctccggtggctaatcaagtagctgatgttgtttctgctaaatctagcaatacccaagagaatgaattgggttcaaagttggggacaggaagcaaaagcaatagggatatggtatttcaaaggtatatttacagatcctggatcacattttgtcaggttctgcttctttattcgtaattgtcaaagtccttaagttcactacttgaatatttgaggccaatgtaatttccattccaacggacgtatgctgcccaggtgtgactactattctgatttatgcttacggcttcgttacatcttggaatcttgcttttagcctgcttatggattttatttataaattgaaagctctgatactaacaaataacagcaccagcacataattgccgtaaatggactgtactactagcctattaaggttttggtaatatggcaaatgccagaatgaattgcctggagactcaggttaccttggactctgagtattttatgcttataactatttttctaatctattatgatgtataatctatattgaatgcaatgatatcaagttgttggctttcgtagatataaaaataagttttattgggttgatggttatactaaaaagcagtttcattgtagaatacatatcatggttatctctctcggatgttgtagaagtcatgcatcatcacttgcctctaaaatgtattgggtgatctgaaaataaatgactgacatttggttttctccacactatttttgcagactgtgcctgcttgccaaaatctctacaagaactccttcacgtatcactgtctccaatatgataagactttaccttttgatgacgcagttgaatttctaggtggcctttctttatattctcccaaatgaactataagatgacactgaacaaatgacttttggttaagaacgtgtcattagctttctatcattcacttattccaaaagtctaactttcctgatattgcttgtgattttcagagcaatgtgagagacaaggctcgggttctggtgcactgcatgtcaggaaaaaataggtttgaattagttgtatttgttatcctttcccatatgacagaagtttagatttgtttaaccatcctctctaataggttacacttatttattgcaggtctccagctattgttatagcattcttgaatattaaataaatactatagattccctgcagcatgcttacaagtagagtgtgaacataacattttagtatttttcatttttctttgtgcctttcctgtctttctcgtgcacataactgtggctctggattctgcttggatccgtctgctctttcctcttgatggcacttgaattgcctatacttgtgctgttagtgtcatagcttataagataagttttcctacagaataattgctacacaagacttcatcccatagttgataatgggctttactgatgtattgtagttgcagctgtatctgtggcataaaggtgaaggcagtttaatttaagattttatgtagtaccattacgttcatctgttgaggagatctttccaatggcttagagaacttcaaaaattttagcagacataccgtcagttttctgaattagctcccttccctaatcagttatactgtgaaatgaaacttttaatccttcactttgtaataagtaaagacgaccttgatgtagcaagtgtagtgcggtatatccgtgcttttggtctcatttcttgtttcctattttcggttaatatttaattgacagcagttatgtgcagtttttgtaaacaaaatatcttatgcttggtttgatgacaagtaaggaccaaaatgaatgacaactatggatatagaattgatttcatggtaacatcttcttgatgagtatgtactaacatagggaaaatgcacttacaaaaagttatgatgatctacatttctattcaagtattgttcaaatgatgcactccccggaacattttctcccccttgtgaaaatctcctccttgctaaggaaggaggaaaaaaaaaaaaaaaaaaaaaaaaaaaaaaaaaaaaaaaagacccgacccgacccgacccaattacacccctactcaaaataaccaataaataaataatgctattgttttcttttc

SEQ ID NO: 16 - protein, 352 aa, Cucumis melo
MGDPNLSINNNNNTCFSLSPFQHYSSSSSSSSSYNSHYHHDHLFSLSYSNDNNSNTLKNNMTTSHYNPSSSSQVLLPLLSLSPARVEQDHHHHHHQNIIDHDHNIIDYDQNDVTVALHLGLPTPSSSSNNNSDLILRLSSTEISDQEDHTHQLQELSSNNSIASNSNGVNKGQYWIPTPTQILIGPTQFSCPLCFKTFNRYNNMQMHMWGHGSQYRKGPQSLRGTQPTAMLRLPCYCCAIGCRNNIDHPRSKPLKDFRTLQTHYKRKHGMKPFTCRKCGKAFAVRGDWRTHEKNCGKLWHCTCGSDFKHKRSLKDHIKAFGLGHAAYGIDDHHNHHHSFDNEDDDPASDIET